(19) United States
(12) Patent Application Publication
(10) Pub. No.: US 2026/0063622 A1
(43) Pub. Date: Mar. 5, 2026

(54) METHOD, SYSTEM, AND DEVICE FOR ANALYZING CELLULAR PROPERTIES WITH MACHINE LEARNING TO IDENTIFY STATES OF SENESCENCE

(71) Applicant: New York University, New York, NY (US)

(72) Inventors: Michael N. Wosczyna, Scarsdale, NY (US); Sahil Mapkar, South Plainfield, NJ (US)

(21) Appl. No.: 19/270,119

(22) Filed: Jul. 15, 2025

Related U.S. Application Data
(60) Provisional application No. 63/671,334, filed on Jul. 15, 2024.
(60) Provisional application No. 63/761,384, filed on Feb. 21, 2025.

(51) Int. Cl. G01N 33/50; G06V 10/26; G06V 10/30; G06V 10/72; G06V 10/762; G06V 10/764; G06V 10/776; G06V 20/69

(52) U.S. Cl. CPC G01N 33/5023; G01N 33/5061; G06V 10/273; G06V 10/30; G06V 10/72; G06V 10/763; G06V 20/695; G06V 10/764; G06V 10/776; G06V 20/698

(57) ABSTRACT

A system, product, and method for analyzing cellular properties and nuclear geometries with machine learning such as unsupervised learning to identify senescence is described. The system, product, and method may include steps or operations including processing images comprising one or more animal cells; determining altered cellular properties associated with senescence of the one or more animal cells of the processed images by extracting: nuclear properties, non-nuclear properties, and functional properties of the animal cells; and determining senescence of the one or more cells by scoring the respective nuclei using unsupervised learning.

### CROSS REFERENCE TO RELATED APPLICATIONS

This application claims priority to U.S. Provisional Application No. 63/761,384, filed on Feb. 21, 2025, and U.S. Provisional Application No. 63/671,334, filed on Jul. 15, 2024, each of which is incorporated herein by reference in its entirety.

### STATEMENT REGARDING FEDERALLY SPONSORED RESEARCH OR DEVELOPMENT

This invention was made with government support under AG053438 awarded by the National Institutes of Health. The government has certain rights in the invention.

### REFERENCE TO A “SEQUENCE LISTING” SUBMITTED AS AN XML FILE

The present application hereby incorporated by reference the entire contents of the sequence listing xml document named “206256-0107-00US_seq_listing.xml”. The xml file containing the Sequence Listing of the present application was created on Nov. 19, 2025 and is 18,779 bytes in size.

### BACKGROUND OF THE INVENTION

A consequence of aging is the progressive loss of effective tissue regeneration, which has been linked to the accumulation of senescent cells (SnCs) (Tuttle, C. S. L. et al. Aging Cell 19, e13083 (2020)). Cellular senescence, or the permanent exit from the cell cycle, typically results from intrinsic damage and is an evolutionary mechanism that limits the propagation of pathological and precancerous cells (Wang, L. et al. Nat. Rev. Cancer 22, 340-355 (2022)). While SnCs exit the cell cycle, they remain metabolically active, releasing inflammatory cytokines as part of the senescence-associated secretory phenotype (SASP) (Coppé, J.-P. et al. Pathol.: Mech. Dis. 5, 99-118 (2010)). These cytokines can stimulate the immune system to aid tissue repair but can also cause chronic inflammation if SnCs accumulate, as in aging (Hu, L. et al. Front. Cell Dev. Biol. 10, 822816 (2022).).

Determining whether SnCs are beneficial or detrimental is likely founded in the contextual influences of the tissue. However, reports vary on whether these cells benefit or hinder similar processes in identical tissues (Rhinn, M. et al. Development 146, dev151837 (2019), Moiseeva, V. et al. Nature 613, 169-178 (2023)). These dichotomies are due to confounding factors like the difficulty in reliably identifying SnCs and the heterogeneity within this population. Many of the markers used to suggest a cell has senesced (e.g., p16, p21, HMGB1, and LaminB1) demonstrate great variability, differing in assays and from study to study. A leading biomarker for SnCs known as senescence associated-β-gal (SA-β-gal) relies on their increased lysosomal density (Valieva, Y. et al. Diagnostics 12, 2309 (2022)). However, even these assays can have high variability due to its dependence on strict pH control. Complicating matters, the limited number of SnCs in tissue makes it challenging to refine assays for consistent and conclusive results.

Some machine learning approaches have been attempted. In Heckenbach, systems were trained on SnCs, but this previous approach did not effectively define conclusive senescence morphologies resulting in weak correlation in many aspects (Heckenbach, I. et al. Nat. Aging 2, 742-755 (2022)). For example, predicted correlation was low until confidence thresholds were set to 90%. It is also known that the unrealized complexity, known as the black box problem, of some learning models (e.g., some supervised learning) may raise reliability concerns. Ultimately, Heckenbach notes that the low performance depends on the training contexts.

Duran paradigmatically approaches the problem from the same angle (Duran, I. et al. Nat. Commun. 15, 1041 (2024)). Duran identifies SnCs using supervised learning approaches, which are dependent on the quality of the training datasets, and the reliability of the training data is context driven. Duran trained on induced senescent cells, and these algorithms were predictive/correlative based on training. This approach also compares induced senescence to uninduced cells, which encounter a similar black box problem as mentioned above which may lead to inaccurate predictions depending on cellular context. The authors in Duran noted that their cell senescent score (CSS) had low consistency and could not be used to predict senescence accurately at the single-cell level.

Therefore, while supervised learning has attempted to be used to identify SnCs consistently, there still exists a need in the art to maintain context independence while also being able to identify SnCs consistently.

### SUMMARY OF THE INVENTION

The present disclosure relates generally to analyzing cellular properties with machine learning to identify senescence, and more particularly, to a product, system, and method for analyzing cellular nuclear geometries with machine learning to identify senescence.

Some embodiments of the invention disclosed herein are set forth below, and any combination of these embodiments (or portions thereof) may be made to define another embodiment.

A device, method, and system for analyzing cellular properties with machine learning to identify senescence is broadly described. The device may include a nontransitory computer readable medium storing instructions that, when executed by one or more processors, causes one or more processors to perform one or more operations. The system may include one or more processors and a memory storing instructions that, when executed by the one or more processors perform one or more operations.

In a first aspect, the invention broadly described may include the steps or operations of: processing images comprising one or more animal cells; determining nuclear morphometrics associated with senescence of the one or more animal cells of the processed images by extracting phenotypes of respective nuclei of the one or more animal cells. The phenotypes may be associated with at least: a geometry of the respective nuclei and a nucleic acid organization. The step or operations may include determining senescence of the one or more animal cells by using the extracted phenotypes using unsupervised learning.

In a second aspect, the invention is broadly described and may include the steps or operations of: processing images comprising one or more animal cells; determining altered nuclear morphometrics associated with senescence of the one or more animal cells of the processed images by extracting: (i) nuclear size, (ii) DAPI intensity, (iii) dense foci, and (iv) circularity of respective nuclei of the one or more animal cells; and determining senescence of the one or more cells by scoring the respective nuclei using unsupervised learning.

In a third aspect, the invention is broadly described and may include the steps or operations of: processing images comprising one or more animal cells; determining altered cellular properties associated with senescence of the one or more animal cells of the processed images by extracting: nuclear properties, non-nuclear properties, and functional properties of the animal cells; and determining senescence of the one or more cells by scoring the respective nuclei using unsupervised learning.

In one or more aspects, one or more animal cells may be in a tissue that include cartilage or muscle.

In one or more aspects, unsupervised learning include dimensional reduction; cluster analysis; and/or Uniform Manifold Approximation and Projection (UMAP).

In one or more aspects, senescence may be determined in vivo where the nucleic acid organization may include light microscopy.

In one or more aspects, altered nuclear morphometrics may include one or more of: nuclear size, DAPI intensity, circularity, blebbing, and dense foci.

In one or more aspects, one or more animal cells may include a sample selected from one of the group of: a cell culture, a histology sample, a tissue sample, a primary cell sample, or an explant culture. In another aspect, the one or more animal cells may include at least one of: endothelial cells, immune cells, stem cells, mesenchymal stem cells, mesenchymal stromal cells, skeletal muscle cells, fibroadipogenic progenitor cells, cancer cells, and tumor cells.

In one or more aspects, processed images may include removing noise from the images using 3D deconvolution. In another aspect, processed images may include removing irregularities.

In one or more aspects, extraction may include generating a binary image from the processed images to detect the respective nuclei.

In one or more aspects, determining the senescence may include a senescence gradient, and the senescent gradient may be based on UMAP.

In one or more aspects, determining may include discrete groups, and the discrete groups may be based on k-means cluster analysis.

In one or more aspects, senescence may be determined ex vivo, wherein the nucleic acid organization may be measured using a nucleic acid marker.

In one or more aspects, nucleic acid marker may include a DAPI marker.

In one or more aspects, nucleic acid organization may include at least one of: dense foci and DAPI intensity.

In one or more aspects, geometry of the respective nuclei may include one or more of: a size, a circularity, a blebbing of the respective nuclei.

In one or more aspects, functional properties of the animal cells may include protein and gene expression.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A to FIG. 1M relate to an aspect of the invention showing nuclear morphometrics that define senescence and can be reduced to a unifying score.

FIG. 1A is a schematic of the nuclear morphometric pipeline (NMP) developed to identify senescence.

FIG. 1B shows a representative H2O2 treatment of C2C12s induces a senescent phenotype in a dose-dependent manner. n=3.

FIG. 1C shows a representative FACS histogram for Spider (SA)-β-Gal staining. The graph quantifies replicates relative to untreated and unstained. n=3.

FIG. 1D shows a representative SASP assessment by qRT-PCR. White indicates equivalency to untreated. n=4.

FIG. 1E shows representative images and quantification of the nuclear morphology between treatment groups. Scale bar—50 μm. n=3 with 4000 to 8000 cells/replicate.

FIG. 1F shows a representative UMAP of reduced single-cell nuclear morphology with 5 groups resolved by the k-means clustering algorithm. Inserts are representative nuclei from each cluster. Scale bars—5 μm. Orange and red clusters exhibit a strong senescence phenotype and are quantified in the bar graph. n=3. Further reduction creates a one-dimensional senescent “score” correlating with nuclear phenotype and treatment condition. n=2000 cells per condition.

FIG. 1G shows a representative quantification of senescent-associated markers in clusters identified as senescent by the NMP. n=3.

FIG. 1H shows representative UMAPs and a line graph quantifying the decrease in SnCs with increasing senolytic concentration, as detected by the NMP, and violin graphs to visualize the decrease in density of low scores that reflect a senescent phenotype. n=3.

FIG. 1I shows an exemplary method 110 for processing images to gather nuclear morphometric properties.

FIG. 1J shows an exemplary method 120 to aid in identifying binary objects, which may include nuclei for example.

FIG. 1K shows an exemplary method 130 relating to an aspect of the invention.

FIG. 1L shows a diagram relating to an aspect of the invention.

FIG. 1M shows an exemplary method 140 relating to an aspect of the invention.

FIG. 2A to FIG. 2H relate to an aspect of the invention for the NMP that identifies a dynamic state of senescence in regenerating skeletal muscle.

FIG. 2A shows a representative schematic demonstrating the experimental approach. FIGS. 2B-D: FAPs, FIGS. 2E-G: SCs.

FIG. 2B and FIG. 2E show representative UMAPs reducing nuclear phenotypes for the noted experimental conditions and the NMP-derived senescent score. Representative associated heat map displays phenotypic differences at noted days post injury (DPI). Representative associated violin plot visualizes differences in score. FAPs, n=1.5-5.0×103 cells per condition; SCs, n=1.0-1.2×103 cells per condition.

FIGS. 2C and 2F show a representative UMAPs defining the age-dependent cell representation in the senescent population. Circle plots quantify the relative representation of cells per age in the SnC cluster.

FIGS. 2D and 2G show representative line graphs showing the age-dependent changes for the noted DPI and the bar graphs denote those of 3 DPI. Significance is relative to untreated. n=3.

FIG. 2H shows a representative visualization of which cell populations contribute to total senescence during skeletal muscle injury at different age groups. n=3 per age.

FIG. 3A-FIG. 3D relate to an aspect of the invention showing a mechanism in which the NMP clusters and scores nuclei using nuclear morphometrics. Data is a revisualization of FIGS. 1E and F.

FIG. 3A depicts a representative expanded correlogram that visualizes histograms of each parameter to dictate a shift of phenotypes found in senescence with increasing H2O2 treatments. Two-way interaction between parameters using scatterplot shows increased density with senescent phenotype and increasing H2O2 treatments. Quantifications use Pearson R coefficients to demonstrate strength of interaction between parameters.

FIG. 3B depicts representative clustering methods including Elbow and Silhouette plots, each of which resolves to 5 clusters.

FIG. 3C depicts a representative visualization of how UMAPs colocalize measured parameters related to senescent phenotype. Larger areas within pie charts (left) and yellow arrows (right) indicate greater measures.

FIG. 3D shows representative line graphs visualizing the strength of score vs. each parameter, demonstrating that a low score correlates with the senescent phenotype.

FIG. 4A-FIG. 4D relate to an aspect of the invention showing a visualization of nuclear phenotypes after in vitro senescence induction.

FIGS. 4A-C show representative images demonstrating senescence. Scale bars-100 μm, inset scale bars—10 μm. All image Look-Up Tables (LUTs) and exposure times are equivalent. DAPI is less intense with increasing H2O2 (300 μM and 500 μM) treatment due to the induction of the senescence phenotype.

FIG. 4D shows representative UMAPs confirming noted stain and expected correlation with SnC clusters (SnCs=X-Gal+, Ki-67−, γH2A+) identified by the NMP. X-Gal n=60-150 cells, γH2A n=100-300 cells, Ki-67=300-350 cells per condition.

FIG. 5A-FIG. 5H relate to an aspect of the invention showing the NMP identifying a dynamic state of senescence among the cell populations of regenerating skeletal muscle. FIGS. 5A-C Immune, FIGS. 5E-G: Endo.

FIGS. 5A and D show representative UMAPs reducing nuclear phenotypes for the noted experimental conditions and the NMP-derived senescent score. Representative associated heat map displays phenotypic differences at noted days post injury (DPI). Representative associated violin plot visualizes differences in score. Immune, 200 cells per condition; Endo—Endothelial, 200 cells per condition.

FIGS. 5B and E show representative UMAPs to demonstrate the identification of senescence.

FIGS. 5C and F show representative quantification by % cells detected as senescent in different time points and age conditions. n=3.

FIG. 5G shows representative quantification of senescence relative to FAPs and quantification of proportion of senescence by cell population between age groups.

FIG. 5H shows representative FACS plots demonstrating gating strategy to isolate cell populations.

FIG. 6 shows representative images of DAPI stained nuclei of different cell types labeled with senescence score generated by the NMP. Scale bars—10 μm.

FIG. 7A-FIG. 7C relates to an aspect of the invention showing confirmation of NMP in Primary Cells.

FIG. 7A shows a representative schematic demonstrating the process of cell harvest to senescence identification.

FIG. 7B and C show representative images of the noted cells for EdU staining with quantification demonstrating differences between aged and young. Scale bars—100 μm. n=4. Representative three-dimensional UMAPs. SCs n=4.0-6.3×104 nuclei per condition. FAPs n=1.4-2.2×104 cells per condition. 4.5×103 nuclei were stochastically chosen and plotted in reduced UMAPs to increase EdU+ cell visualization.

FIG. 8A-FIG. 8E relate to an aspect of the invention showing the NMP Successfully Identifies SnCs in an Osteoarthritic Model.

FIG. 8A shows a representative schematic demonstrating the process of cartilage extraction to senescence identification.

FIG. 8B shows a representative histological images of Safranin O staining (SO, red area) to visualize arthritic changes in cartilage that occur with aging. Note the loss of SO staining in the geriatric samples. Scale bar—200 μm.

FIGS. 8C and D show representative UMAPs of the noted cell populations from the NMP demonstrating identification of senescence. Chondrocytes n=82-96 cells per condition, Immune n=123-134 cells per condition. Violin plots reveal the differences in senescent scores. Quantification by % cells detected as senescent in different ages. n=5.

FIG. 8E show representative FACS plots demonstrating gating for isolation of associated cell populations. Note, immune cells come from bone underlying the articular cartilage, but not bone marrow, since Ter119 staining is negative.

FIG. 9 depicts one aspect of the invention showing an exemplary computing system.

FIG. 10A-FIG. 10E depict senescent conversion altering nuclear morphology. FIG. 10A depicts a schematic of the nuclear morphometric pipeline (NMP) developed to identify senescence (Created in Biorender. Mapkar, S. (2025) BioRender(dot)com/w2xaf4w). FIG. 10B depicts H2O2 treatment of C2C12 cells induces a senescent phenotype in a dose-dependent manner. MFI—mean fluorescence intensity. n=3 independent experimental replicates. FIG. 10C depicts a representative FACS histogram for Spider SA-β-Gal staining. The graph quantifies replicates relative to untreated and unstained. Blue—untreated, Red—300 μM, Orange—500 μM. n=3 independent experimental replicates. FIG. 10D depicts SASP assessment by qRT-PCR. n=4 independent experimental replicates, where each column represents one replicate. Green—increased expression, Orange—decreased expression, White—equivalency to untreated. FIG. 10E depicts representative images and quantification of the nuclear morphology between treatment groups. Note, image Look-Up Tables (LUTs) and exposure times are equivalent. Scale bar—50 μm. n=3 independent experimental replicates. Error bars represent the mean±standard error of the mean (SEM), and statistical tests were conducted using unpaired two-sided t tests with no adjustments. *p≤0.05, **p<0.01, ***p<0.001, ****p<0.0001. Precise p-values are listed in Table 3.

FIG. 11 depicts representative high-magnification images of DAPI stained H2O2 treated C2C12 nuclei from non-SnCs and SnCs. Top—non-SnC, Bottom—SnC. Large nuclear images are at the level noted by a red box in Z-stack images that are shown in the black-and-white panels. Volumetric representations of nuclei for each cell type are shown in the 3D panels. Variable LUTs were needed for clarity. Scale bars—10 μm. Table of parameters (right) diagram the 4 nuclear morphometrics used in the NMP (Created in BioRender. Mapkar, S. (2025) BioRender(dot)com/qmm2ysb).

FIG. 12A-FIG. 12E shows nuclear morphometrics define senescence and can be reduced to a unifying score. FIG. 12A depicts a UMAP of reduced single-cell nuclear morphology with 5 groups resolved by the k-means clustering algorithm. Inserts are representative nuclei from each cluster of H2O2 treated C2C12 cells. Image Look-Up Tables (LUTs) and exposure times are equivalent. Scale bars—5 μm. FIG. 12B depicts further dimensional reduction creating a one-dimensional senescent “score” correlating with nuclear phenotype and treatment condition. FIG. 12C depicts a bar graph quantifying senescence phenotype and showing that orange (senescent) and red (senescent like) clusters exhibit a strong senescence phenotype. n=3 independent experimental replicates. FIG. 12D depicts a quantification of senescent-associated markers in clusters identified as senescent by the NMP. MFI—mean fluorescence intensity; NS—non-senescent; SEN—senescent. n=3 independent experimental replicates. FIG. 12E depicts Representative UMAPs and a line graph quantifying the decrease in SnCs with increasing senolytic concentration, as detected by the NMP. Gray—untreated, Blue—300 μM, Green—500 μM. Violin plots visualize senescent score (y-axis) to demonstrate decrease in the density of low-scoring, morphologically senescent nuclei, as shown inside the red box. Comparisons are made within treatment groups. n=3 independent experimental replicates. Error bars represent the mean±standard error of the mean (SEM), and statistical tests were conducted using unpaired two-sided t tests with no adjustments except for (E), which used a two-way ANOVA.*p≤0.05, **p<0.01, ***p<0.001. Precise p-values are listed in Table 3.

FIG. 13A-FIG. 13D show the Mechanism in which the NMP clusters and scores nuclei using nuclear morphometrics. Data is a revisualization of FIG. 12A and FIG. 12B. FIG. 13A depicts an expanded correlogram visualizing histograms of each parameter to dictate a shift of phenotypes found in senescence with increasing H2O2 treatments. Two-way interaction between parameters using scatterplot shows increased density with senescent phenotype and increasing H2O2 treatments. Quantifications use Pearson R coefficients to demonstrate strength of interaction between parameters. FIG. 13B depicts clustering methods including Elbow and Silhouette plots, each of which resolves to 5 clusters. FIG. 13C depicts a visualization of how UMAPs colocalize measured parameters related to senescent phenotype. Larger areas within pie charts (left) and yellow arrows (right) indicate greater measures. FIG. 13D depicts line graphs visualizing the strength of score vs. each parameter, demonstrating that a low score correlates with the senescent phenotype. Error bars represent the mean±standard error of the mean (SEM) and statistical tests were conducted using unpaired two-sided t-tests with no adjustments. ***p<0.001, ****p<0.0001.

FIG. 14A-FIG. 14F shows scores for NMP clusters correlate with senescent markers. FIG. 14A depicts a demonstration of the theoretical relation between nuclear morphological associated score (purple) and an associated senescent marker (orange). FIG. 14B depicts a revisualization of C2C12s treated with H2O2 as in FIG. 12D. FIG. 14C depicts a revisualization of C2C12s treated with etoposide as in FIG. 15E. FIG. 14D depicts a revisualization of C2C12s treated with doxorubicin as in FIG. 3F. FIG. 14E depicts a revisualization of 3T31Ls treated with H2O2 as in FIG. 18A. FIG. 14F depicts a revisualization of 3T31Ls treated with Etoposide as in FIG. 18B. FIG. 14G depicts a revisualization of 3T31Ls treated with doxorubicin as in FIG. 18C. Bar graphs show clusters defined by the NMP (x-axis) with the % stain of each noted marker (left y-axis) and the NMP-defined score (right y-axis).

FIG. 15A-FIG. 15F show that the NMP is applicable across varying mechanisms of senescence induction. FIG. 15A depicts a quantification of senescence markers following etoposide treatment of C2C12s. FIG. 15B depicts a quantification of senescence markers following doxorubicin treatment of C2C12s. FIG. 15C depicts representative images and quantification of nuclear morphometrics of C2C12s treated with etoposide. FIG. 15D depicts representative images and quantification of nuclear morphometrics of C2C12s treated with doxorubicin. Note the dosage response to each small molecule inducer. Exposure times and Look-Up Tables (LUTs) are held constant. Scale bar—200 μm. n=3 independent experimental replicates. FIG. 15E depicts a large UMAP consisting of reduced single-cell nuclear morphology with 5 groups resolved by the k-means clustering algorithm for etoposide. FIG. 15F depicts a large UMAP consisting of reduced single-cell nuclear morphology with 5 groups resolved by the k-means clustering algorithm for doxorubicin. Inserts are representative nuclei from each cluster. Scale bars—20 μm. One-dimensional senescent “score,” in small UMAPs, correlating with nuclear phenotype and treatment condition, shows dose-dependent quantification in violin plots. Orange (senescent) and red (senescent like) clusters exhibit a strong senescence phenotype and are quantified in the horizontal color bar graph. n=3 independent experimental replicates. Gray bar graphs are the quantification of senescent-associated markers in clusters identified as senescent by the NMP. n=3 independent experimental replicates. MFI—mean fluorescence intensity. NS—non-senescent; SEN—senescent. Error bars represent the mean±standard error of the mean (SEM), and statistical tests were conducted using unpaired two-sided t-tests with no adjustments. *p≤0.05, **p<0.01, ***p<0.001, ****p<0.0001. Precise p-values are listed in Table 3.

FIG. 16A-FIG. 16L depict a visualization of nuclear phenotypes after in vitro senescence induction in C2C12 cells. FIG. 16A, FIG. 16B, and FIG. 16C depict representative images demonstrating senescence conversion following treatment with H2O2. FIG. 16D depicts a representative UMAP of H2O2 treated C2C12s. FIG. 16E, FIG. 16F, and FIG. 16G depict representative images demonstrating senescence conversion following treatment with etoposide. FIG. 16H depicts a representative UMAP of etoposide treated C2C12s. FIG. 16I, FIG. 16J, and FIG. 16K depict representative images demonstrating senescence conversion following treatment with doxorubicin. FIG. 16L depicts a representative UMAP of doxorubicin treated C2C12s. Scale bars—100 μm, inset scale bars—10 μm. All image Look-Up Tables (LUTs) and exposure times are equivalent between treatment groups. DAPI is less intense with increasing treatment due to the induction of the senescence phenotype. Representative UMAPs are correlated with staining to confirm SnC clusters are genuinely senescent (SnCs=X-Gal+, Ki-67−, γH2A+). NS—non-senescent; SEN—senescent.

FIG. 17A-FIG. 17G shows the NMP effectively defines SnCs in 3T3-L1 preadipocytes treated with H2O2, etoposide, or doxorubicin. FIG. 17A depicts a SASP assessment by qRT-PCR for 3T3-L1s treated with the noted senescence inducer. White indicates equivalency to untreated. n=3 independent experimental replicates per condition, where each column represents one replicate. FIG. 17B depicts a quantification of senescent phenotype markers in 3T3-L1s treated with H2O2. FIG. 17C depicts a quantification of senescent phenotype markers in 3T3-L1s treated with etoposide. FIG. 17D depicts a quantification of senescent phenotype markers in 3T3-L1s treated with doxorubicin. Note the dose-dependent response to senescence inducers with decreasing Ki67 and increasing γH2A, phenotypic markers of SnCs. MFI—mean fluorescence intensity. n=3 independent experimental replicates per condition. FIG. 17E depicts representative images and quantifications of nuclear morphometrics of nuclei treated with H2O2. FIG. 17F depicts representative images and quantifications of nuclear morphometrics of nuclei treated with etoposide. FIG. 17G depicts representative images and quantifications of nuclear morphometrics of nuclei treated with doxorubicin. Between inducers image Look-Up Tables (LUTs) and exposure times are equivalent. n=3 independent experimental replicates; scale bar—200 μm. Error bars represent the mean±standard error of the mean (SEM) and statistical tests were conducted using unpaired two-sided t-tests with no adjustments. *p≤0.05, **p<0.01, ***p<0.001, ****p<0.0001.

FIG. 18A-FIG. 18F show a visualization of nuclear phenotypes after in vitro senescence induction in 3T3-L1 cells. FIG. 18A depicts a large UMAP of reduced single-cell nuclear morphology with 5 groups resolved by the k-means clustering algorithm for 3T3-L1s treated with H2O2. FIG. 18B depicts a large UMAP of reduced single-cell nuclear morphology with 5 groups resolved by the k-means clustering algorithm for 3T3-L1s treated with etoposide. FIG. 18C depicts a large UMAP of reduced single-cell nuclear morphology with 5 groups resolved by the k-means clustering algorithm for 3T3-L1s treated with doxorubicin. Orange and red clusters exhibit a strong senescence phenotype and are quantified with corresponding colors in the horizontal bar graphs. n=3 independent experimental replicates. Inserts are representative nuclei from each cluster. Scale bars—20 μm. MFI—mean fluorescence intensity. Further reduction creates a one-dimensional senescent “score” correlating with nuclear phenotype (small UMAP) and treatment condition (violin plots). Bar graphs quantify the noted marker in non-SnCs (NS) and SnCs (SEN). n=3 independent experimental replicates. FIG. 18D depicts representative images of cells treated with H2O2 and stained for senescence associated markers. FIG. 18E depicts representative images of cells treated with etoposide and stained for senescence associated markers. FIG. 18F depicts representative images of cells treated with doxorubicin and stained for senescence associated markers. Between inducers image Look-Up Tables (LUTs) and exposure times are equivalent. Corresponding UMAPs confirm expected staining for SnC clusters (SnCs=Ki-67−, γH2A+) identified by the NMP. NS—non-senescent; SEN—senescent. Error bars represent the mean±standard error of the mean (SEM) and statistical tests were conducted using unpaired two-sided t-tests with no adjustments. *p≤0.05, **p<0.01, ***p<0.001, ****p<0.0001.

FIG. 19A-FIG. 19H show the NMP identifies a dynamic state of senescence in regenerating skeletal muscle. FIG. 19A depicts a schematic demonstrating the experimental approach (Created in BioRender. Mapkar, S. (2025) BioRender(dot)com/mxe0n7o). FIG. 19B depicts a representative UMAP reducing nuclear phenotypes for the noted experimental condition in fibroadipogenic progenitors and the NMP-derived senescent score. Representative associated heat map displays phenotypic differences at noted days post injury (DPI), Red—increased from uninjured, Blue—decreased from uninjured. The representative associated violin plot visualizes differences in score. FIG. 19C depicts a representative UMAP defining the age-dependent cell representation in the senescent population of fibroadipogenic progenitors. Circle plots quantify the relative representation of cells per age in the SnC cluster. Blue—young, Red—aged, Green—geriatric. FIG. 19D depicts a line graph showing the age dependent changes for the noted DPI of fibroadipogenic progenitors, and the bar graph denotes those of 3 DPI. Significance is relative to the untreated. n=3 independent biological replicates per age. Teal—Uninjured, Purple—3 DPI, Orange—21 DPI. FIG. 19E depicts a representative UMAP reducing nuclear phenotypes for the noted experimental condition in satellite cells and the NMP-derived senescent score. Representative associated heat map displays phenotypic differences at noted days post injury (DPI), Red—increased from uninjured, Blue—decreased from uninjured. The representative associated violin plot visualizes differences in score. FIG. 19F depicts a representative UMAP defining the age-dependent cell representation in the senescent population of satellite cells. Circle plots quantify the relative representation of cells per age in the SnC cluster. Blue—young, Red—aged, Green—geriatric. FIG. 19G depicts a line graph showing the age dependent changes for the noted DPI of satellite cells, and the bar graph denotes those of 3 DPI. Significance is relative to the untreated. n=3 independent biological replicates per age. Teal—Uninjured, Purple—3 DPI, Orange—21 DPI. FIG. 19H depicts a visualization of which cell populations contribute to total senescence during SkM injury at different age groups. Enriched Endothelial Cells—Enr. ECs. Immune Cells—ICs. All UMAP cell numbers from each condition are equivalent. Error bars represent the mean±standard error of the mean (SEM), and statistical tests were conducted using unpaired two-sided t-tests with no adjustments. **p<0.01, ***p<0.001, ****p<0.0001. Precise p-values are listed in Table 3.

FIG. 20A-FIG. 20H shows the NMP identifies a dynamic state of senescence among the cell populations of regenerating SkM. FIG. 20A depicts a representative UMAP reducing nuclear phenotypes for the noted experimental conditions in immune cells and the NMP-derived senescent score. Representative associated heat map displays phenotypic differences at noted days post injury (DPI). Representative associated violin plot visualizes differences in score. FIG. 20B depicts a representative UMAP to demonstrate the identification of senescence in immune cells. FIG. 20C depicts a quantification in immune cells by % cells detected as senescent in different time points and age conditions. n=3 independent biological replicates. FIG. 20D depicts a representative UMAP reducing nuclear phenotypes for the noted experimental conditions in enriched endothelial cells and the NMP-derived senescent score. Representative associated heat map displays phenotypic differences at noted days post injury (DPI). Representative associated violin plot visualizes differences in score. FIG. 20E depicts a representative UMAP to demonstrate the identification of senescence in enriched endothelial cells. FIG. 20F depicts a quantification in enriched endothelial cells by % cells detected as senescent in different time points and age conditions. n=3 independent biological replicates. FIG. 20G depicts a quantification of senescence relative to FAPs and quantification of proportion of senescence by cell population between age groups. Fibroadipogenic progenitors—FAPs, Satellite Cells—SCs. FIG. 20H depicts representative FACS plots demonstrating gating strategy to isolate cell populations; top row is from uninjured SkM while bottom row is from injured. Error bars represent the mean±standard error of the mean (SEM) and statistical tests were conducted using unpaired two-sided t-tests with no adjustments. *p≤0.05, **p<0.01, ***p<0.001, ****p<0.0001.

FIG. 21A-FIG. 21B depict a visualization of sorted primary cells and their associated senescence score. FIG. 21A depicts representative (60×) high-magnification images of DAPI stained nuclei from non-senescent (Left) and senescent (Right) FAPs of 3 DPI tissue. Red boxed Z-stack image shown in the black-and-white panels is displayed as large nuclear image. Volumetric representations are shown in the 3D panels. Scale bars—10 μm. Image Look-Up Tables (LUTs) are variable to achieve clarity. FIG. 21B depicts representative images of DAPI stained nuclei of the noted cell types labeled with senescence score generated by the NMP. LUTs and exposure times are equivalent for these images. Scale bars—10 μm.

FIG. 22A-FIG. 22F shows the NMP can effectively identify FAPs with senescent characteristics in vivo in the regenerating muscles of young and geriatric mice. FIG. 22A depicts representative images of geriatric skeletal muscle (SkM) cross-sections identifying PDGFRα+ FAPs, Laminin+ encircled muscle fibers, Ki67+ proliferating cells, and DAPI to identify and resolce nuclear morphology. FIG. 22B depicts a quantification of proliferating FAPs relative to young. Violin plots quantify the noted marker per single cell (each dot). MFI—mean fluorescence intensity. n=3 independent biological replicates per age. FIG. 22C depicts a UMAP showing the clustering of single FAPs by the NMP with senescent scores indicated by color (lower scores—purple—are highly senescent cells), and expression level of Ki67 by the size of the circle. Bar graphs quantify the amount of Ki67 in the non-SnC (NS) or SnCs (SEN). FIG. 22D depicts representative images of geriatric skeletal muscle (SkM) cross-sections identifying PDGFRα+ FAPs, Laminin+ encircled muscle fibers, γH2A+ damage, and DAPI to identify and resolce nuclear morphology. FIG. 22D depicts a quantification of proliferating FAPs relative to young. Violin plots quantify the noted marker per single cell (each dot). MFI—mean fluorescence intensity. n=3 independent biological replicates per age. FIG. 22E depicts a UMAP showing the clustering of single FAPs by the NMP with senescent scores indicated by color (lower scores—purple—are highly senescent cells), and expression level of γH2A by the size of the circle. Bar graphs quantify the amount of Ki67 in the non-SnC (NS) or SnCs (SEN). n=3 independent biological replicates per age. Scale bars—50 μm, inset scale bars—10 μm. Error bars represent the mean±standard error of the mean (SEM) and statistical tests were conducted using unpaired two-sided t-tests with no adjustments. *p≤0.05, **p<0.01, ***p<0.001. Precise p-values are listed in Table 3.

FIG. 23A-FIG. 23F shows The NMP identifies endothelial cells (ECs) with senescent characteristics in vivo in the regenerating muscles of young and geriatric mice. FIG. 23A depicts representative images of geriatric SkM cross-sections identifying CD31+ endothelial cells, γH2A, and DAPI to quantify nuclear morphology. FIG. 23B depicts a quantification of γH2A+ damaged endothelial cells relative to young. Violin plots quantify the noted marker per single cell (each dot). MFI—mean fluorescence intensity. n=3 biological independent replicates per age. FIG. 23C depicts a UMAPs showing the clustering of ECs by the NMP with senescent scores indicated by color (lower scores are highly senescent cells), and expression level of the noted marker by the size of the circle. FIG. 23D depicts representative images of geriatric SkM cross-sections identifying CD31+ endothelial cells, Ki67, and DAPI to quantify nuclear morphology. FIG. 23E depicts a quantification of Ki67+ proliferating endothelial cells relative to young. Violin plots quantify the noted marker per single cell (each dot). MFI—mean fluorescence intensity. n=3 biological independent replicates per age. FIG. 23F depicts a UMAPs showing the clustering of ECs by the NMP with senescent scores indicated by color (lower scores are highly senescent cells), and expression level of the noted marker by the size of the circle. n=3 biological independent replicates per age. Bar graphs quantify the amount of the noted marker in the non-SnC (NS) or SnCs (SEN). Scale bars—50 μm, inset scale bars—10 μm. Error bars represent the mean±standard error of the mean (SEM) and statistical tests were conducted using unpaired two-sided t-tests with no adjustments. *p≤0.05, **p<0.01, ***p<0.001.

FIG. 24 shows The NMP identifies FAPs and endothelial cells (ECs) with senescent characteristics in vivo in young regenerating muscles. FIG. 24A depicts representative images of young skeletal muscle (SkM) cross-sections identifying PDGFRα+ FAPs and Laminin+ encircled muscle fibers. FIG. 24B depicts representative images of young skeletal muscle (SkM) cross-sections identifying CD31+ endothelial cells, stained with (top) γH2A, or (bottom) Ki67. Scale bars—50 μm, inset scale bars—10 μm.

FIG. 25 shows In vivo visualization of nuclei at 3 DPI in SkM tissue determined to be senescent (SEN) and non-senescent (NS) by the NMP. FIG. 25A depicts PDGFRα+ FAPs. FIG. 25B depicts CD31+ Endothelial cells. Top—Representative three-dimensional high-magnification images of DAPI stained nuclei from. Bottom—Single image from Z-stack three-dimensional images. Scale bars—5 μm. Image Look-Up Tables (LUTs) are variable to achieve clarity and exposure times are equivalent.

FIG. 26A-FIG. 26C shows the NMP effectively identifies SnCs from primary cells grown in culture. FIG. 26A depicts a schematic demonstrating the process of cell harvest to senescence identification (Created in BioRender. Mapkar, S. (2025) BioRender(dot)com/mj1i554). FIG. 26B and FIG. 26C depict representative images of the noted cells, stained for EdU incorporation, with bar graphs quantifying differences between young and geriatric cells for proliferation (% EdU+), % SnCs as defined by the NMP, and % EdU+ in NMP-defined non-senescent cells (NS) and senescent cells (SEN). Image Look-Up Tables (LUTs) and exposure times are equivalent. Scale bars—200 μm. n=4 independent biological replicates per age. Representative UMAPs. Error bars represent the mean #standard error of the mean (SEM) and statistical tests were conducted using unpaired two-sided t-tests with no adjustments. *p≤0.05, **p<0.01, ***p<0.001, ****p<0.0001.

FIG. 27A-FIG. 27E shows senolysis of SnCs in freshly sorted primary cells confirms the efficacy of the NMP in detecting genuine senescence. FIG. 27A depicts a schematic for the experimental approach, from cell harvest to senescence identification (Created in BioRender. Mapkar, S. (2025) BioRender(dot)com/vrb4qji). FIG. 27B depicts representative images and quantification demonstrating dose response of senolytic treatment removing NMP-defined SnCs in sorted FAPs collected from injured muscle. Image Look-Up Tables (LUTs) and exposure times are equivalent. Violin graphs visualize the decrease in density of cells with low scores that reflect SnC presence. Scale bars—50 μm. n=8 experimental replicates with cell input pulled from 2 mice to make 4 replicates, this was done twice. FIG. 27C depicts a representative UMAP of FAPs with the senescence “score” derived from the NMP and nuclei noted per the treatment condition. Donut charts plot the relative contribution of each treatment group (by color) to the senescent or non-senescent clusters. FIG. 27D depicts representative images and quantification demonstrating dose response of senolytic treatment removing NMP-defined SnCs in sorted SCs collected from injured muscle. Image Look-Up Tables (LUTs) and exposure times are equivalent. Violin graphs visualize the decrease in density of cells with low scores that reflect SnC presence. Scale bars—50 μm. n=7 experimental replicates with cell input pulled from 2 mice to make 4 replicates, and 2 mice to make 3 replicates. FIG. 27E depicts a representative UMAP of SCs with the senescence “score” derived from the NMP and nuclei noted per the treatment condition. Donut charts plot the relative contribution of each treatment group (by color) to the senescent or non-senescent clusters. Significance is relative to the untreated group. Error bars represent the mean #standard error of the mean (SEM) and statistical tests were conducted using unpaired two-sided t-tests with no adjustments. *p≤0.05, **p<0.01, ***p<0.001, ****p<0.0001.

FIG. 28A-FIG. 28E show the NMP successfully identifies SnCs in an osteoarthritic model. FIG. 28A depicts a schematic demonstrating the process of cartilage extraction to senescence identification (Created in BioRender. Mapkar, S. (2025) BioRender(dot)com/5gviyx6). FIG. 28B depicts representative histological images of Safranin O staining (SO, red area) to visualize arthritic changes in cartilage that occur with aging. Note the loss of SO staining in the geriatric samples. Scale bar—200 μm. FIG. 28C and FIG. 28D depict a representative UMAP of the noted cell populations from the NMP demonstrating identification of senescence. Violin plots reveal the differences in senescent scores. Quantification by % cells detected as senescent in different ages. n=5 independent biological replicates per age. FIG. 28E depicts representative FACS plots demonstrating gating for isolation of associated cell populations. Note, immune cells come from bone underlying the articular cartilage, but not bone marrow, since Ter119 staining is negative. Error bars represent the mean±standard error of the mean (SEM) and statistical tests were conducted using unpaired two-sided t-tests with no adjustments. *p≤0.05, ****p<0.0001.

FIG. 29A-FIG. 29B show NMP-defined SnCs from cartilage have a senescent phenotype. FIG. 29A depicts bar graphs quantifying differences between young and geriatric chondrocytes for proliferation (% Ki67+). FIG. 29A also depicts Representative images of young and geriatric cells stained for Ki67. FIG. 29B depicts bar graphs quantifying differences between young and geriatric chondrocytes for DNA damage (γH2A). % SnCs as defined by the NMP. % Ki67 or mean florescence intensity (MFI) of γH2A in NMP-defined non-senescent cells (NS) and senescent cells (SEN). FIG. 29B also depicts Representative images of young and geriatric cells stained for γH2A. Image Look-Up Tables (LUTs) and exposure times are equivalent. Scale bars—50 μm. n=3 independent biological replicates per age, each with 100-200 nuclei from chondrocytes quantified per condition. Error bars represent the mean±standard error of the mean (SEM) and statistical tests were conducted using unpaired two-sided t-tests with no adjustments.*p≤0.05, **p<0.01, ***p<0.001.

FIG. 30 shows the NMP integrates biological, computational, and graphical components to extract and classify nuclear morphologies associated with cellular senescence. Nuclear phenotypic features, including area, circularity, intensity, and foci, are extracted from images acquired using the Nikon AR system (light blue) yielding raw quantitative data imported into RStudio (purple). Following normalization (red), the dataset undergoes unsupervised learning through two parallel approaches: K-means clustering to identify discrete nuclear phenotypes (yellow), and Uniform Manifold Approximation and Projection (UMAP) for dimensional reduction (green). The 2D UMAP output facilitates graphical visualization of nuclear phenotypic space, while the 1D embedding provides a continuous “senescence score” reflecting nuclear deviation from non-senescent morphology (blue). Final outputs include graphical cluster representation and senescence scores enabling robust detection and quantification of senescent cells across diverse biological conditions. (Created in BioRender. Mapkar, S. (2025) BioRender(dot)com/mzmqzsj).

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The terminology used herein is for the purpose of describing particular example embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. That is, terms such as “first,” “second,” and other numerical terms, when used herein, do not imply a sequence or order unless clearly indicated by the context. Thus, a first element, component, region, layer or section discussed below could be termed a second element, component, region, layer or section without departing from the teachings of the exemplary embodiments.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Embodiments are provided throughout so that this disclosure is sufficiently thorough and fully conveys the scope of the disclosed embodiments to those who are skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that embodiments may be embodied in different forms. As such, the embodiments should not be construed to limit the scope of the disclosure. As referenced above, in some embodiments, well-known processes, well-known device structures, and well-known technologies may not be described in detail.

Inconsistencies in identifying cellular senescence have led to varying conclusions about the impact of this cell fate in regenerative and aging scenarios. However, due to the cell cycle component of senescence, altered nuclear morphologies have been suggested as an alternative means to identify SnCs, characteristics that require simple reagents to resolve and can be detected in minimal cells, thus providing consistent categorization of SnCs (Huang, W. et al. Nat. Rev. Nephrol. 18, 611-627 (2022), Freyter, B. M. et al. Cells 11, 273 (2022), Zhang, R. et al. Dev. Cell 8, 19-30 (2005)). In experimental examples, this nuclear morphometric pipeline (NMP) is translated to detect SnCs in young, aged, and geriatric skeletal muscle (SkM) and cartilage, two tissues reported to contain SnCs, proving its efficacy in detecting in vivo senescence (Wan, M. et al. Bone Res. 9, 41 (2021), Saito, Y., et al. Nat. Commun. 11, 889 (2020), Yagi, M. et al. Sci. Rep. 13, 7697 (2023)). In these examples, the system reveals an age-dependent dynamic milieu of bona fide SnC populations in these tissues and can be readily deployed with consistent nuclear assessments. A robust pipeline discussed below in detail is established using nuclear morphometrics to identify bona fide senescence induced in culture as well as other contexts using machine learning techniques.

Techniques that have utilized supervised learning to identify senescent cells are highly context driven as the ultimate dataset depends on the training context (Moiseeva, V. et al. Nature 613, 169-178 (2023), Duran, I. et al. Nat. Commun. 15, 1041 (2024)). Supervised learning techniques may also not be able to give insight into the specific and definable nuclear morphometric properties, variances of which are directly related to cell senescence. Additionally, these techniques can perform poorly in determining senescence accurately at the single cell level or in cell samples in which senescent cells are not enriched. These techniques may also need more specific methods of cell staining before imaging which can limit the contexts in which the technique can be applied.

The strengths of the pipeline discussed below include the use of an unsupervised learning algorithm which can perform across different cell types and tissue types without concern for inherent characteristics driving senescence classification. In some embodiments, the pipeline can accurately generate a senescence score for individual cells within a sample that can reflect a gradient from non-senescent to pre-senescent to genuine senescence in addition to accurately identifying the general senescence load in a sample. In some embodiments, the pipeline can accurately identify senescent cells in contexts not enriched for senescent cells. The pipeline may obtain images of cells stained by simple cell staining techniques that can be used in diverse contexts. The pipeline may rely on dimensional reduction to identify senescent cells which has high resolution and is amenable to visual representation.

The nuclear morphometrics used for the NMP were identified for their direct relationship to cell cycle dynamics and senescence being a phenotype of permanent exit in the G1 or G2 phase (Coryell, P. R. et al. Nat. Rev. Rheumatol. 17, 47-57 (2021)). In fact, nuclear size and DAPI intensity have been connected to cell cycle phases (Roger, L., et al. Int. J. Mol. Sci. 22, 13173 (2021)). These characteristics are associated with senescence, presumably due to the sustained p53 expression and subsequent deregulation of mTOR, and the formation of actin stress fibers causing nuclear flattening in SnCs (Ferro, A. et al. Lab. Investig. 97, 615-625 (2017), Manohar, S. et al. Mol. Cell 83, 4032-4046.e6 (2023)). Additionally, nuclear blebbing leading to decreased circularity in SnCs may be a result of lamin B1 degradation, and heterochromatic foci appearing as punctate nuclear densities in SnCs are associated with highly methylated DNA regions caused by the increased expression of cycle arrest regulators, such as E2F (Maskey, R. S. et al. Cell Cycle 20, 65-80 (2021), Matias, I. et al. Aging Cell 21, e13521 (2022), Romanov, V. S. et al. Cell Cycle 9, 3945-3955 (2010), Pospelova, T. V. et al. Methods Mol. Biol. (Clifton, NJ) 965, 383-408 (2013)).

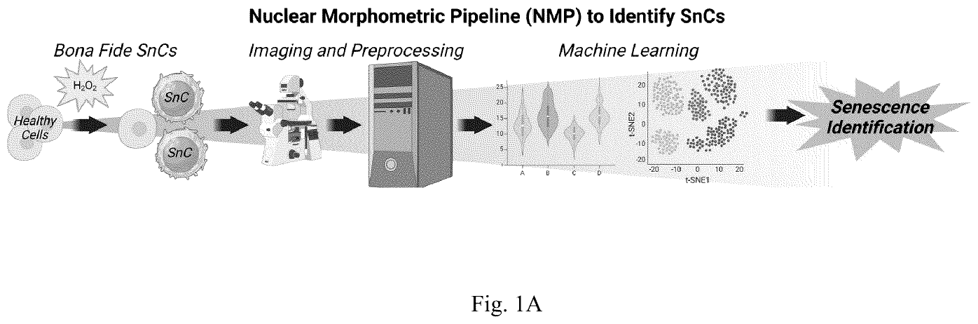

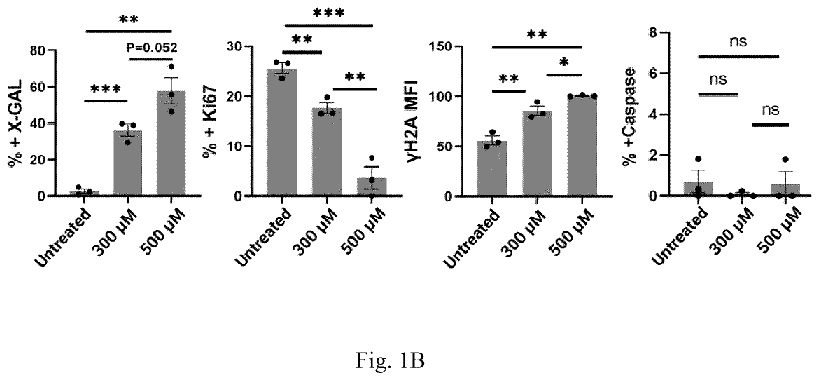

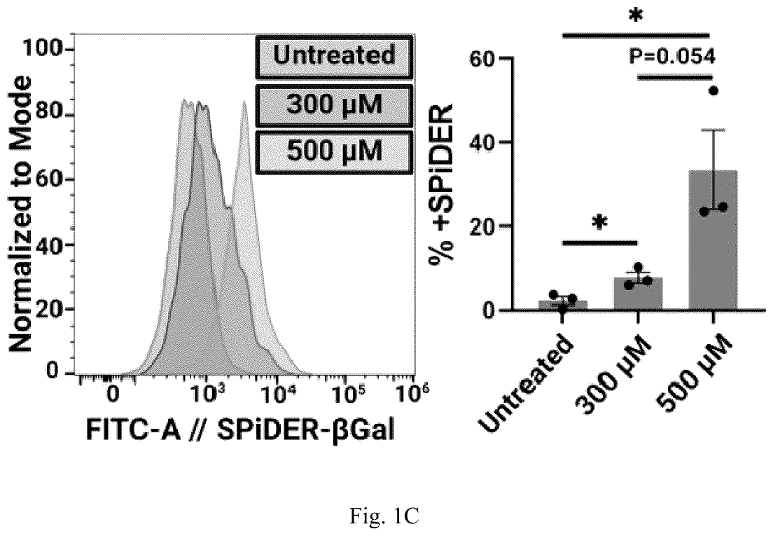

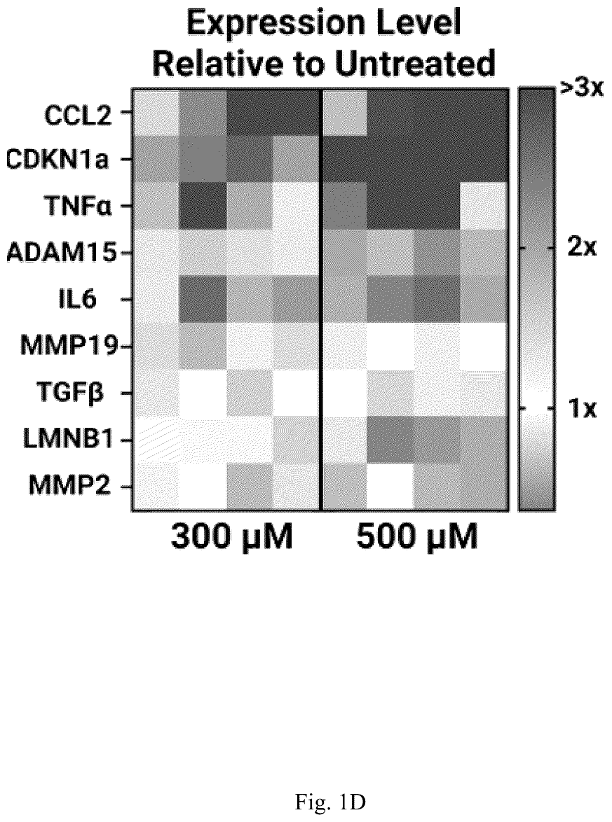

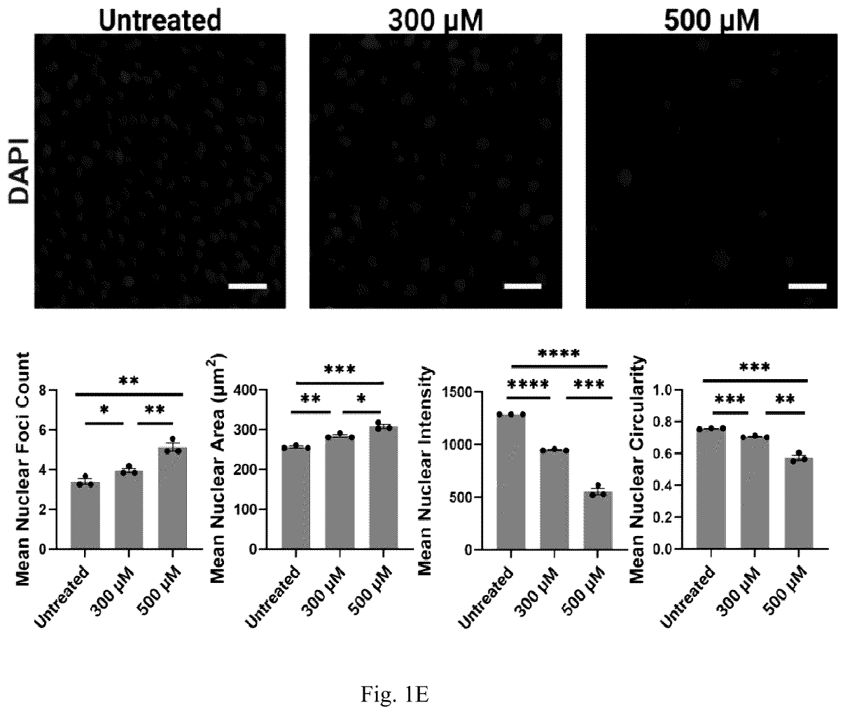

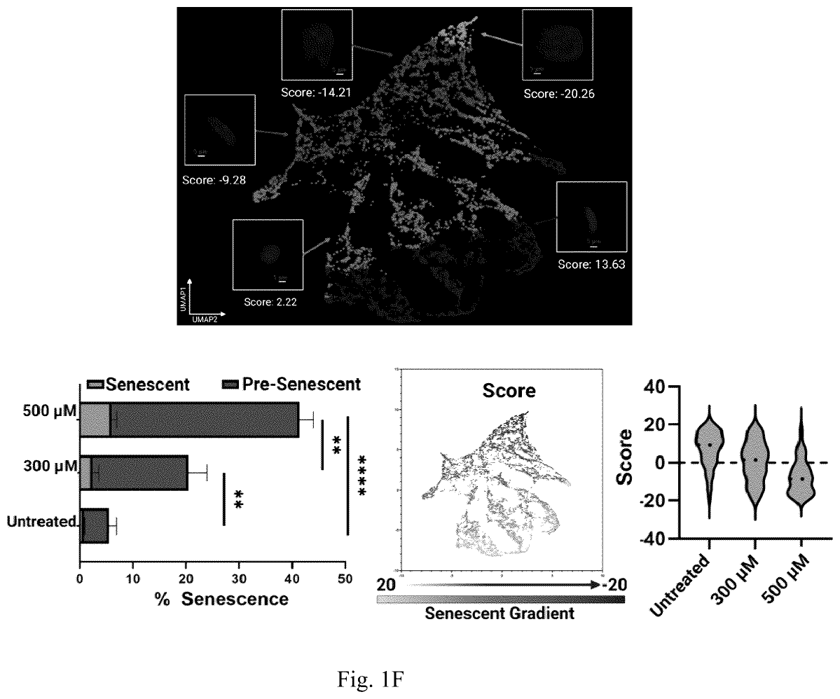

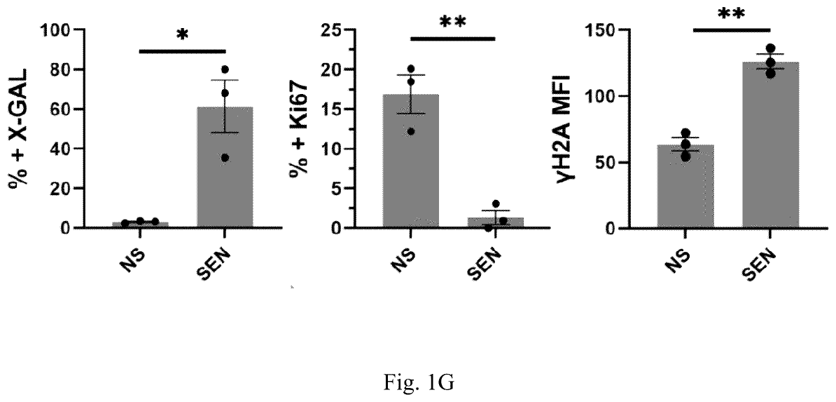

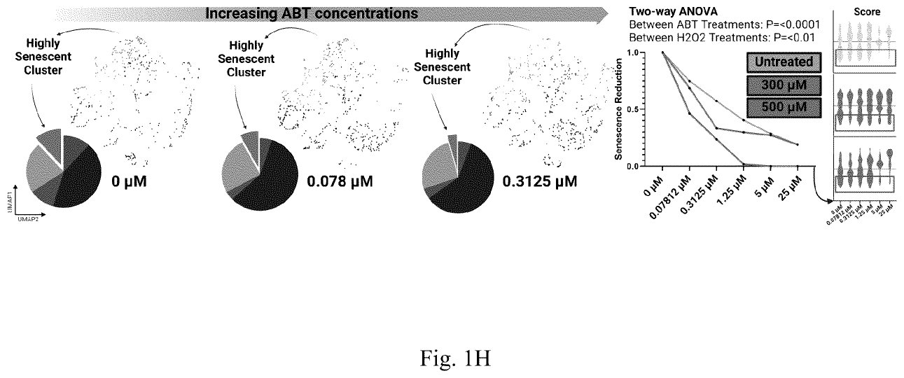

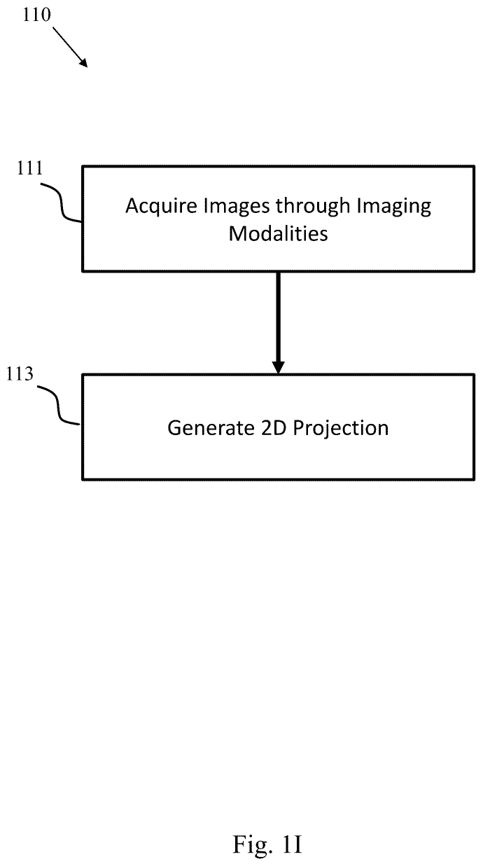

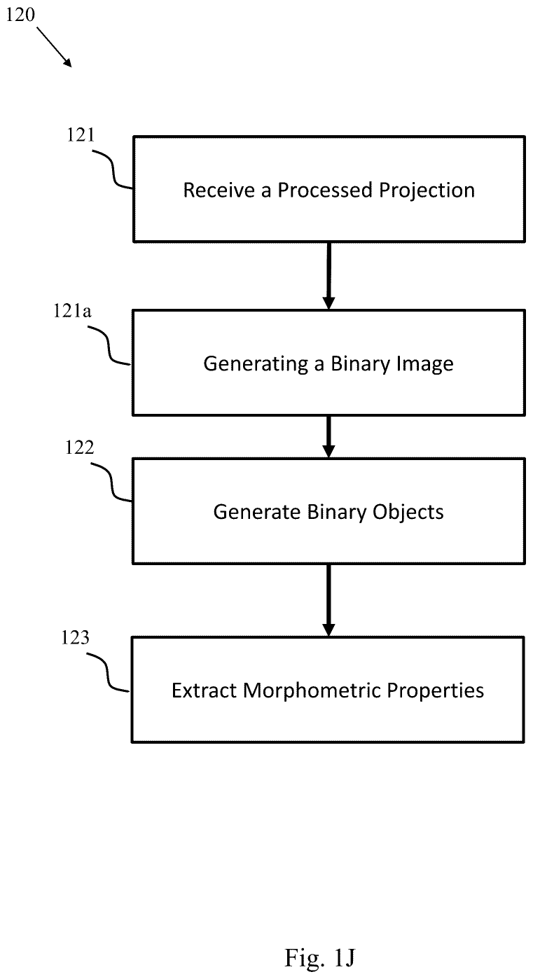

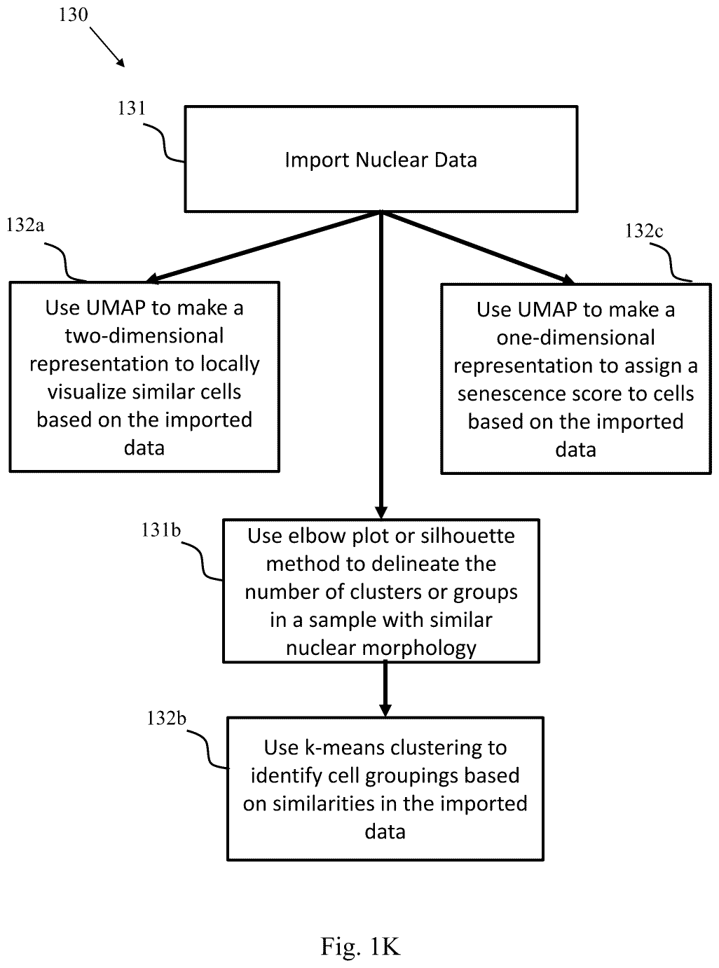

Referring now to FIG. 1A, shown is an exemplary NMP that relates to an approach that quantifies and reduces nuclear morphometrics to reliably identify SnCs, thereby circumventing many of the problematic assays currently used. The ease of use of the NMP, along with its consistency, provides a means of greater standardization for SnC identification. With this will come a better understanding of the biology of SnCs in endogenous processes, such as regeneration and aging, ultimately helping define better targeting of these cells for therapeutics. For example, senescence of cells within a culture sample can be recorded in control samples and in samples after treatment to discern the effects of a treatment. In another example, senescence of cells in samples derived from animal models can be compared. Animal models can include control or wild type animal models, animal disease models, animal models treated with a therapeutic, genetically engineered animal models or any other animal model. In another example, senescence of cells in samples derived from human subjects can be recorded and compared before and after a treatment to discern the effects of genetic differences, a therapeutic, or other biological processes including aging on tissues. Human subjects can include healthy subjects, aged and young subjects, control and treatment groups, or any other human subject.

Accordingly, the disclosed method uses four morphological changes (increase in nuclear size and densefoci, decrease in nuclear circularity and DAPI intensity) to identify bona fide SnCs (FIG. 1E). Additional methods may be used including High Content Analyses (HCA) with Exploratory Factor Analyses (EFA), Scree plot, and Lasso regression that may identify further morphological changes to be considered in identifying SnCs. Each of the morphometrics can independently present as a cell begins to acquire damage and initiate its path to senescence, as demonstrated by the visualized “gradient” in the NMP results (FIG. 1F). This trajectory of senescence reveals an interesting concept that may be used in some senolytics methods to target cells earlier in the transition, potentially improving the limited efficacy of senescence therapies (Narita, M. et al. Cell 113, 703-716 (2003)). For example, the distribution of senescence scores of cells in a sample can be assessed to discern the effect of a treatment. In another example, cells in a sample can be identified or grouped into any number of senescence subcategories by unsupervised machine learning clustering methods or defined by senescence score, and the percent or number of cells in each subcategory or specific subcategories of interest can be compared across samples to discern the effect of a treatment.

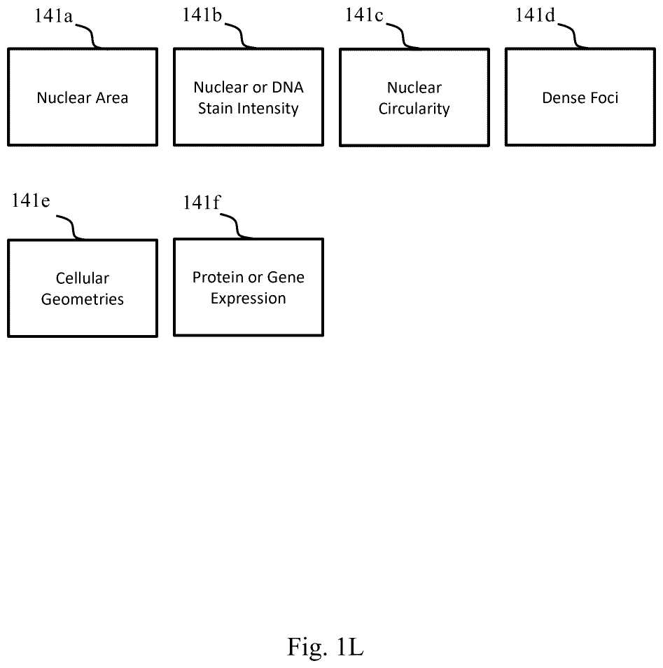

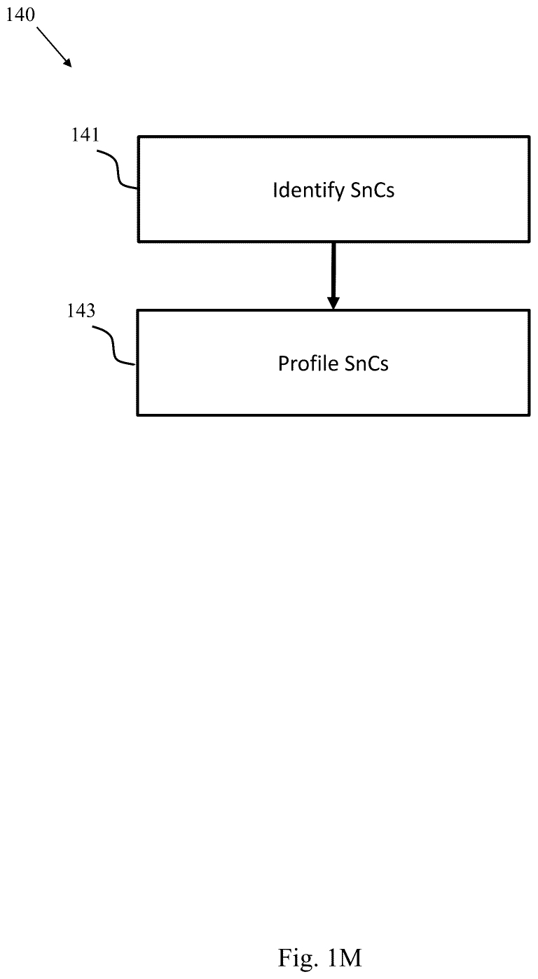

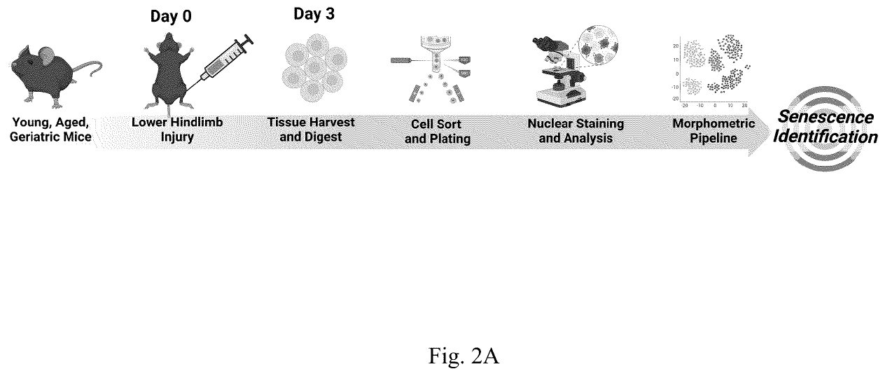

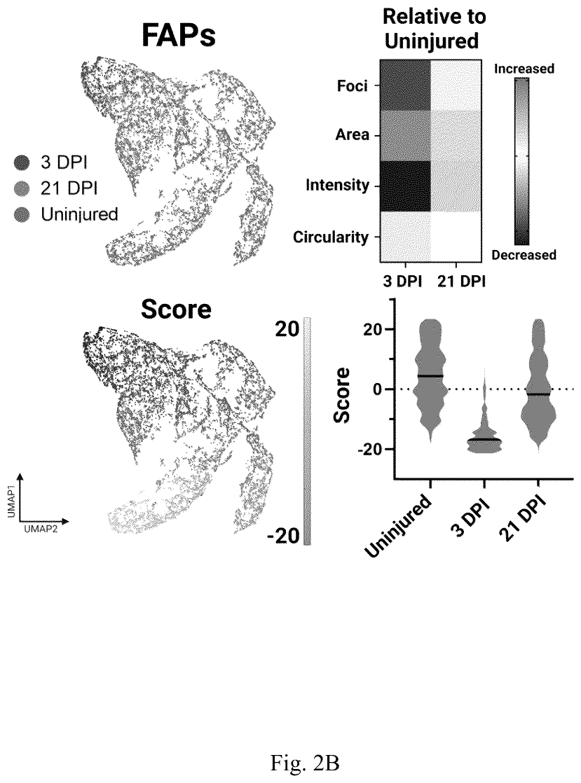

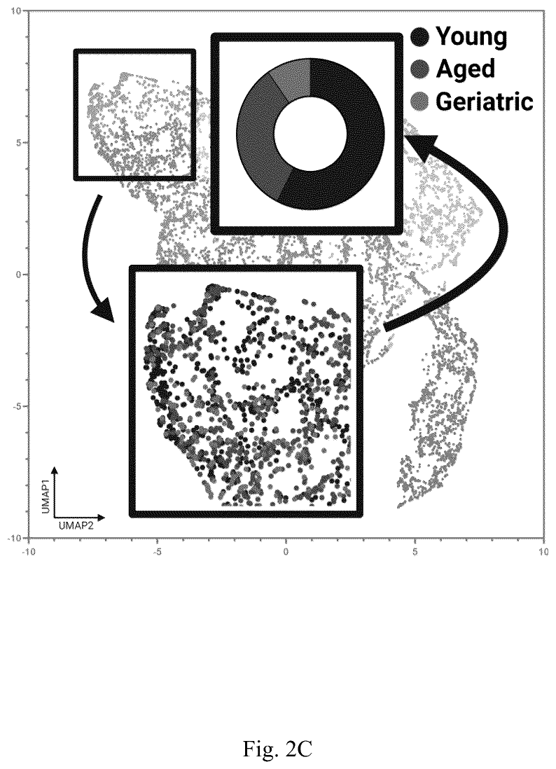

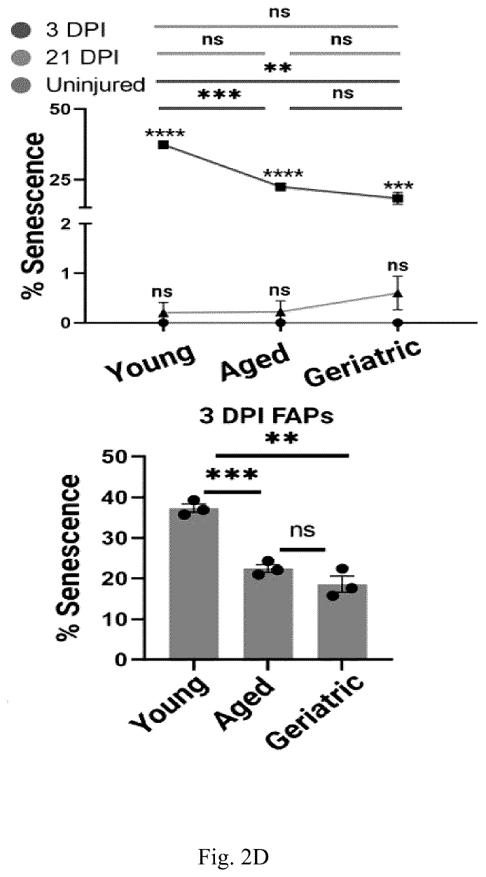

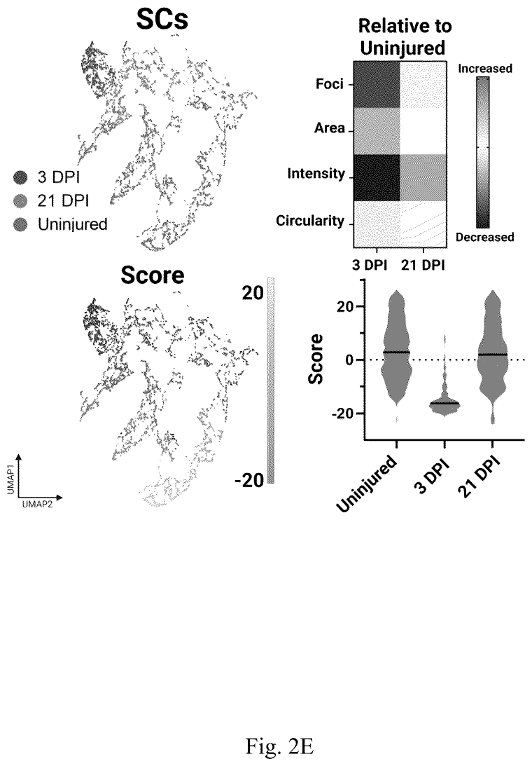

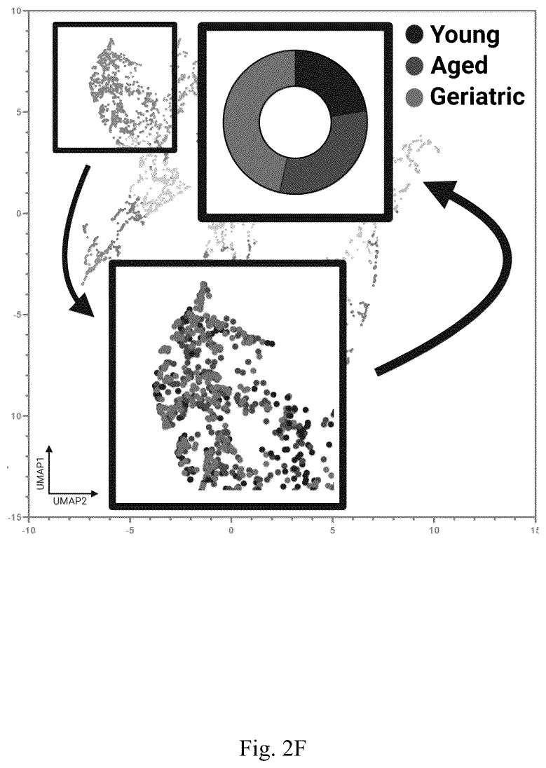

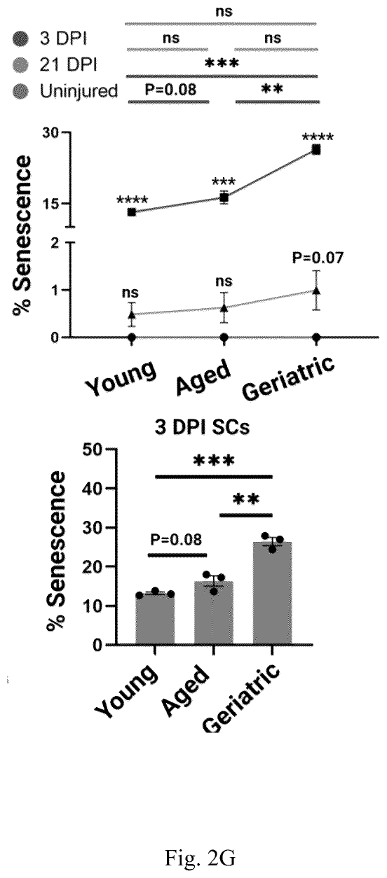

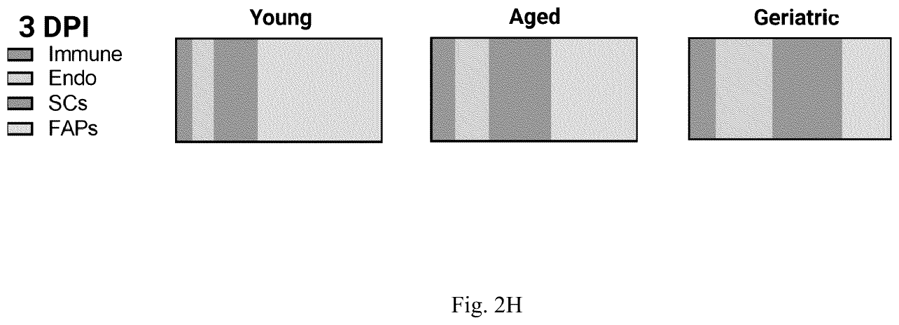

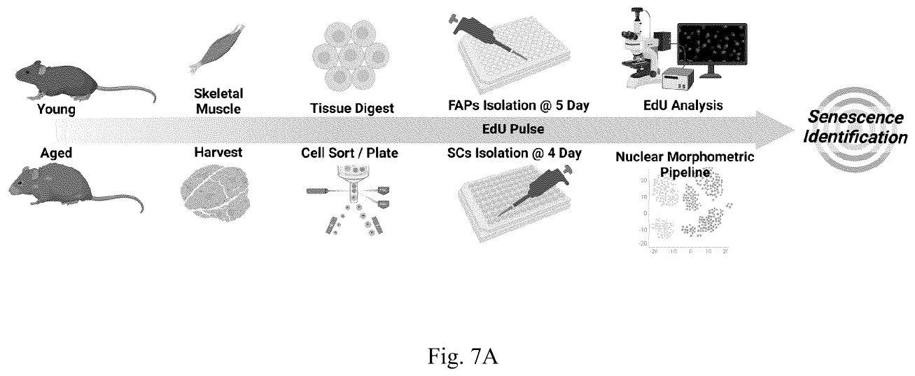

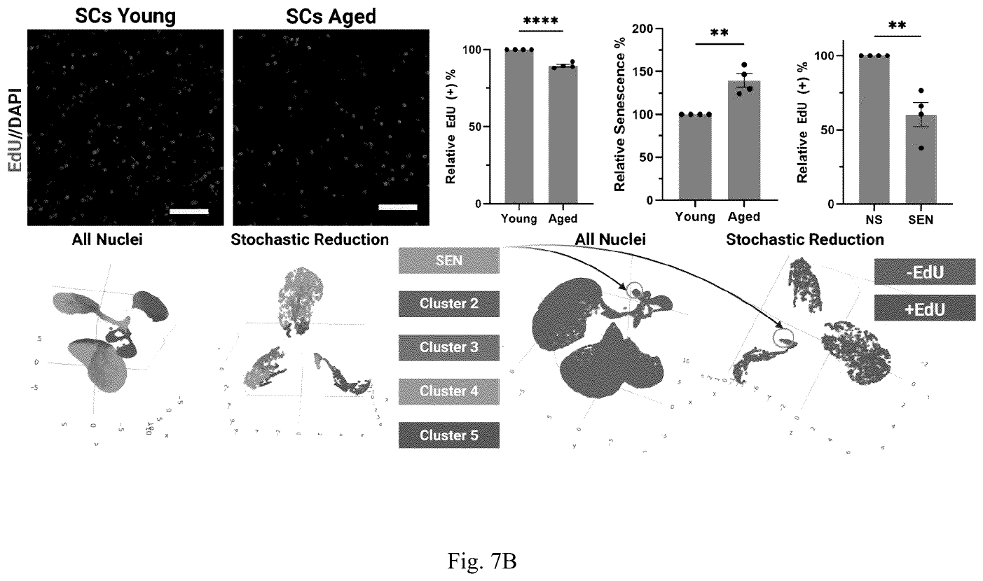

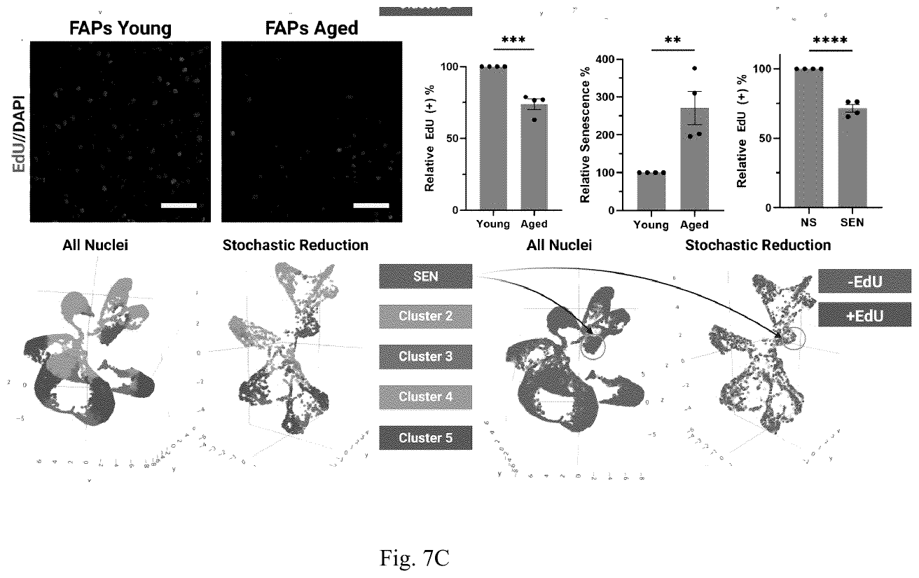

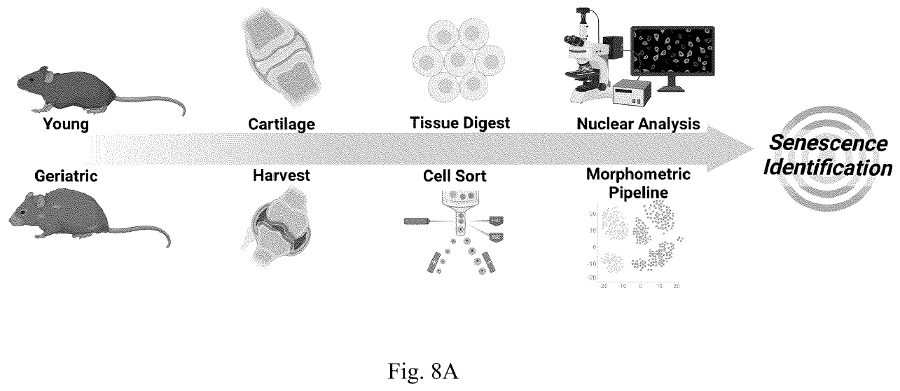

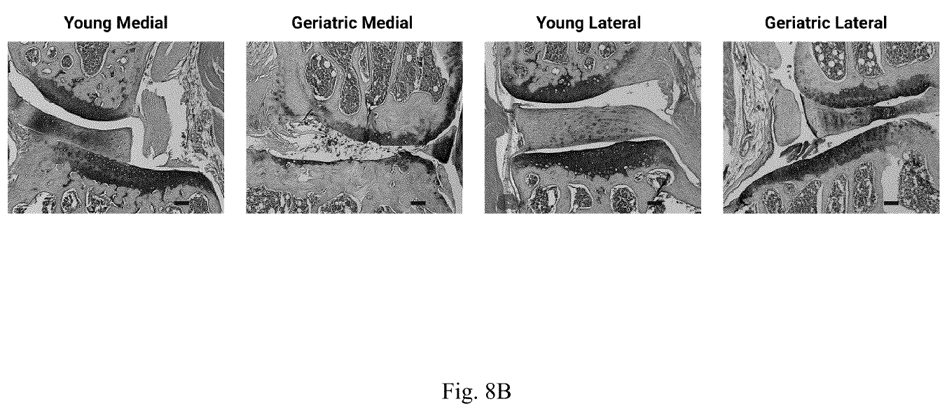

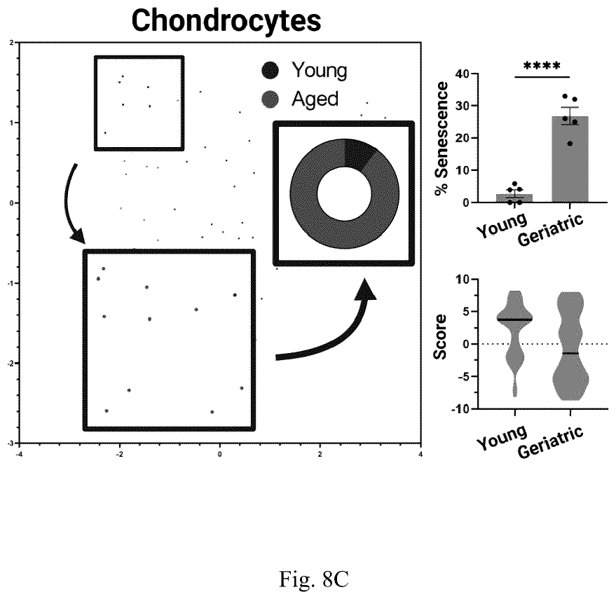

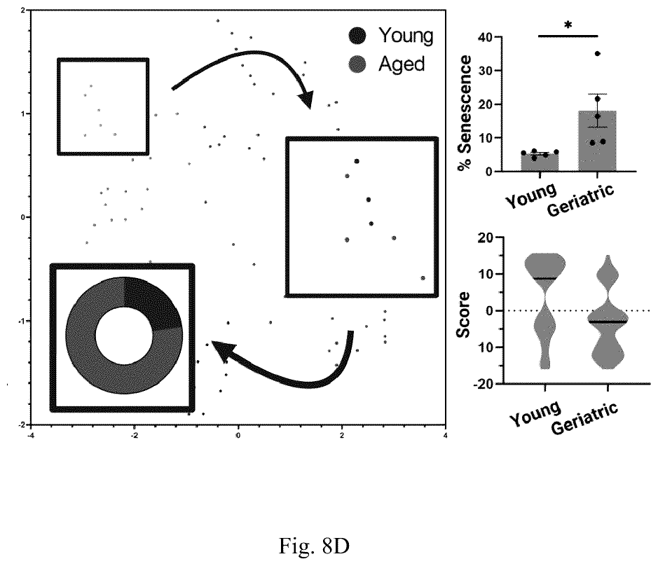

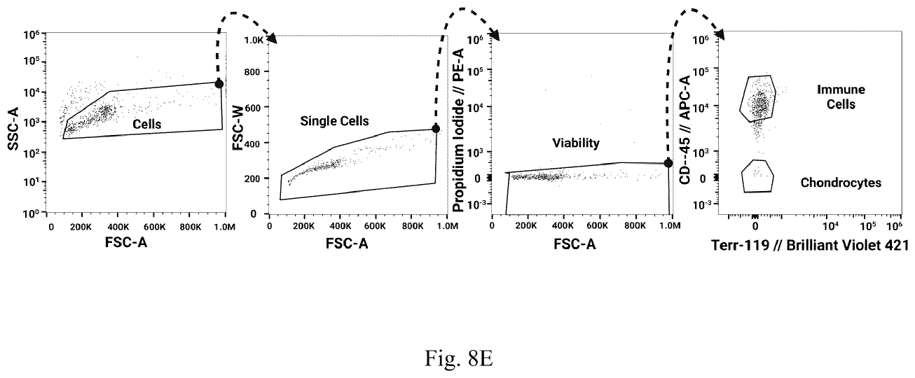

The application of the NMP in SkM homeostasis and regeneration in young, aged, and geriatric mice revealed minimal SnCs in homeostatic tissue, but a robust SnC expansion following injury in all ages (FIGS. 2A-2H). The regenerative SnC expansion in young mice was mainly composed of FAPs at a time coinciding with their peak proliferative expansion,³³ supporting their necessity for efficient regeneration potentially through the SASP-dependent regulation of the immune cell response (Wosczyna, M. N. et al. Cell Rep. 27, 2029-2035.e5 (2019)). Interestingly, FAPs from aged uninjured tissue grown in culture for 5 days were more senescent than those from young tissue, indicating aged cells are sensitive to replicative senesce, while young cells may be more responsive to stress-induced senescence of the in vivo regenerative milieu (compare FIG. 7C to FIG. 2C, D). However, regeneration in aged environments have altered cellular compositions of SnCs, with SCs and ECs becoming the foremost components. These data support the detrimental role of SC senescence in aged SkM, as this has been shown to limit the stem cell pool that directly contributes cells for the rebuilding muscle fibers and the unfavorable inflammatory profile of senescent ECs, as a potential source of chronic inflammation in aging (Chikenji, T. S. et al. EBioMedicine 44, 86-97 (2019), Sousa-Victor, P. et al. Nature 506, 316-321 (2014), Zeng, W. et al. Dev. Cell 58, 1383-1398.e6 (2023)). While the data does not show a drastic senescence shift in ICs from young to geriatric age, the presence of immune SnCs can have a compounding detrimental effect on their inherent ability to clear SnCs (Grosse, L. et al. Cell Metab 32, 87-99.e6 (2020)). Further use of the NMP in age-associated primary osteoarthritis in mice clearly defines senescent chondrocytes in geriatric articular cartilage (FIGS. 8A-8E). Similar to SCs in SkM, senescence in chondrocytes contributes to joint pathology by reducing their functional capacity (decreased ECM production) and likely by promoting a chronic inflammatory environment through SASP expression, which negatively impacts non-SnCs (Saito, Y. et al. Nat. Commun. 11, 889 (2020)).

Collectively, these data demonstrate that the NMP can be implemented in different environments across ages and translated to many tissues to define SnCs. The intriguing dynamic representations of SnCs revealed by using the NMP reinforce the importance of considering the age of an individual when attempting to clear SnCs with an approach that does not discriminate between their different origins, as one may be beneficial and the other detrimental.

Referring now to FIG. 1I, shown is an exemplary method 110 for processing images to gather nuclear morphometric properties. The method may begin with step 111 by acquiring the images via any suitable imaging modality. In some embodiments, fluorescence microscopy and/or brightfield microscopy may be used. In some embodiments, time-lapse microscopy may be used for imaging live cells. For the purposes of imaging live cells, a transgene may be used in an animal model or a plasmid may be used in cultured cells such that the cells in either case express a fluorescent protein or label that localizes to the nucleus for the purposes of enhancing imaging live cells. Fluorescent proteins or labels that can be used may include but are not limited to histones tagged with fluorescent proteins including H2B-GFP or H2B-RFP. In some embodiments, microscopes including a widefield microscope, a confocal microscope, a spinning disk confocal microscope, a scanning confocal microscope, a light microscope, or a light sheet microscope may be used.

The acquired images may be of any cell culture or tissue sample. For example, the cell culture may comprise any cell line or be derived from any primary cells including patient cells. The cell culture may comprise a 2D cell culture, a 3D cell culture, an organoid, an explant culture, and/or an adhered culture. In some examples, the tissue sample of an acquired image comprises a histological sample for example a tissue slice prepared for histology by any method known in the art.

In some embodiments, the images may be acquired at one focal length. In one embodiment, multiple focal lengths spanning a Z-height may be acquired, also known as a z-stack, by confocal fluorescence microscopy or widefield fluorescence microscopy. The step size between multiple focal lengths (optical slices) may include any values in the range of 0.1 μm-10 μm (e.g., 1 μm). The range of the z-stack may in some embodiments span the height of a cell or more than the height of the cell. In various embodiments, the cells imaged may be stained with any number of nuclear or DNA stains to provide an enhanced definition of nuclei. For example, fluorescence microscopy can be used to acquire z-stack images of a cell culture that may be stained with DAPI.

In step 113, a 2D projection of the acquired optical slices may be used. The 2D projection of the z-stack may be a max projection, an average projection, etc. The projection generation may perform processing, which may include preprocessing or postprocessing. As part of preprocessing, it can be appreciated that the 2D projection may have noise removed from the z-stacked image using a 3D deconvolution. Similarly, post-processing of the 2D projection may include removing unwanted nuclei in images or overlapping nuclei from multiple cells.

In various embodiments, and as appreciated by the skilled artisan, other image processing techniques may be used in both pre- or post-processing stages. Such examples include (i) low pass filtering may be used to allow details larger than a set pixel value or to remove small irregularities, (ii) binning, which may reduce file size or improve signal to noise ratio (iii) methods to increase contrast, (iv) subtracting the background (e.g., using a rolling ball function), and (v) denoising pixels with a local linear regression algorithm that compares neighboring pixels from adjacent frames on a 3D axis.

Referring now to FIG. 1J, shown is an exemplary method 120 to aid in identifying binary objects, which may represent nuclei for example. In step 121, the method may begin with receiving a processed projection. It can be appreciated by a person of ordinary skill that, while the processed projection includes the nucleus (whereby nuclear properties 141a-d may be extract as shown in FIG. 1L), the acquired image may also contain corresponding brightfield images or other fluorescence channel images containing images from which other cellular data can be gathered including protein expression, gene expression, protein post-translational modifications, cell granularity, or cell geometries such as the area of the whole cell, cell flattening and the like, each shown in 141e and/or 141f discussed below. This is the case as a person of ordinary skill would understand that staining with DAPI does not exclude the ability to use other stains or dyes or other techniques including brightfield imaging, immunofluorescence microscopy, and in situ hybridization.

Next, the method 120 may include step 121a generating a binary image. A binary image may be generated from the processed cell image from the method 110 discussed above and may be generated by using a threshold pixel intensity value, manual annotation, automated annotation, or a combination of manual and automated annotation. However, in one embodiment, step 121 may proceed directly to generating binary objects in 122 as manual annotation, automated annotation, or a combination of manual and automation may be used to directly define objects. Additionally, some software may perform binary image generation in the background.

In step 122, automated annotation of the processed image or the binary image may be used to identify binary objects representing respective nuclei of multiple cells in the image. In some embodiments, automated annotation may be accomplished by first detecting edges or otherwise segmenting the image into a set of objects, then calculating an approximate size and/or circularity of each of the set of objects, then removing objects that fall below or above predetermined thresholds for size and/or circularity. In some embodiments, all objects with size less than 25 μm² or greater than 600 μm² may be removed. In some embodiments, alternate lower bounds may include 15 μm², 20 μm², 30 μm², 35 μm², or 40 μm². In some embodiments, alternate upper bounds may include 500 μm², 550 μm², 625 μm², 650 μm², or 700 μm². In some embodiments, all objects with circularity less than 0.1 may be removed. In other embodiments, all objects with circularity less than 0.2, 0.18, 0.15, 0.12, or 0.08 may be removed. In some embodiments, the user may manually correct by filtering or removing binary objects that were falsely identified as singular nuclei and may also manually correct by adding binary objects to represent nuclei that were not identified automatically. In some embodiments, High Content Analyses (HCA) may be used to examine additional characteristics of binary objects.

In step 123, morphometric properties shown in FIG. 1L may be extracted from the remaining binary objects. The morphometrics may include nuclear morphometrics such as 141a-d discussed below in detail. In some embodiments, the morphometrics may include additional cellular data as shown in 141e. Additionally, other cellular data may be extracted including gene or protein expression 141f data, discussed below in detail.

Referring now to FIG. 1K, shown is an exemplary method 130 for identifying SnCs by collecting at least nuclear morphometrics data and using machine learning (e.g., unsupervised learning) to group cells or score cells by assigning a score associated with senescence properties based on nuclear morphometrics data. Identified cell groupings may include SnCs, non-senescent cells, novel senescence phenotypes, subcategories of SnCs, cells that will become senescent, cells that have a high likelihood of becoming senescent, cells that will transition out of senescence, cells that were once senescent, cells responding to a treatment condition related to senescence, apoptotic cells, cycling cells, proliferating cells, viable cells, or differentiating cells.

The method may begin with step 131 by importing cellular data. In one embodiment, cellular data may include data about one or more nuclei. In one embodiment, cellular data including nuclear morphometric data may be normalized using a z-score within their respective parameters before subsequent machine learning steps. In one embodiment, cellular data including nuclear morphometric data may include data that correlates with or is indicative of DNA damage, DNA methylation, euchromatin, heterochromatin, or other DNA and nuclear properties. In one embodiment, the imported cellular data comprises number of dense foci, nuclear area, intensity of a nuclear or DNA stain, and/or nuclear circularity. Imported cellular data may also be used secondarily to probe, confirm, or determine cellular phenotypes or states, for example proliferation, DNA damage, viability, cell type, cell metabolism, senescence-associated secretory phenotype (SASP), disease state, cell rejuvenation, and/or cell fate.

In various embodiments, the cellular data may comprise multiple cellular properties. These properties may include nuclear morphometric properties, non-nuclear morphometric properties, cellular geometries, and protein or gene expression data shown in FIG. 1L. Examples of nuclear morphometric properties may include nuclear area 141a, nuclear or DNA stain intensity 141b, nuclear circularity 141c, and dense foci 141d, which are each discussed below in detail. Other contemplated nuclear morphometric properties include convexity, mean chord length, length, kurtosis, roughness, and mode object intensity. In some embodiments, nuclear circularity may be substituted for nuclear convexity and/or nuclear mean chord length. In some embodiments, nuclear area may be substituted for nuclear length. In some embodiments, dense foci may be substituted for nuclear kurtosis and/or nuclear roughness. In some embodiments, nuclear or DNA stain intensity may be substituted for mode nuclear or DNA stain intensity.

In step 132a, a machine learning algorithm may be used to localize visually similar cells based on the imported cellular data (e.g., nuclear morphology). In one embodiment, cellular data may include nuclear morphometric properties. In one embodiment, the machine learning algorithm of step 132a may include unsupervised machine learning, such as uniform manifold approximation projection (UMAP) to generate a lower-dimensional, for example a 2-dimensional representation of a cell population. In an alternative embodiment, the machine learning algorithm used to generate a 2-dimensional representation of the cell population in step 132a may be t-distributed stochastic neighbor embedding (t-SNE), principal component analysis (PCA), or a combination of such dimensional reduction techniques that use integration anchors defined by the mutual nearest neighbor method.

In one embodiment, an elbow plot or silhouette method are used optionally together to delineate the number of groups or clusters with similar nuclear morphology within a cell population in step 131b. In an alternative embodiment, an elbow plot or silhouette method are not used to delineate the number of groups or clusters with similar nuclear morphology. In one embodiment, in step 132b, k-means unsupervised machine learning can subsequently be applied to define groups with definite nuclear morphometric similarity and/or identify senescent nuclei. In one embodiment, in step 132c dimensional reduction to one dimension using the unsupervised machine learning algorithm UMAP is used. In an alternative embodiment, the machine learning algorithm used to generate a 1-dimensional representation of the cell population in step 132c may be t-distributed stochastic neighbor embedding (t-SNE) or principal component analysis (PCA). In step 132c, a senescence score may be generated based on the one-dimensional representation of the cells, which may subcategorize SnCs or may allow for cells to be placed on a senescence gradient.

In some embodiments, any combination of steps 132a, 132c, and 131b/c can be executed, and the outputs of each step can be visualized together. In some embodiments, k-means clustering is used to define groups of cells that can be further identified as SnCs, non-SnCs, pre-SnCs, other subcategories of SnCs, or subcategories of cell cycle. In some embodiments, the number or percent of cells in a sample assigned to senescence groupings is recorded and used to determine a response to a therapeutic agent, make a diagnosis, or determine other phenotypes of a tissue or sample. In some embodiments, a minimum or maximum senescence score is used to define SnCs, non-SnCs, pre-SnCs, or other subcategories of SnCs. In some embodiments, a senescence score may be taken to refer to a senescence gradient wherein cells can be identified as more or less senescent based on a cell's senescence score. Any number of senescence score ranges may be defined to identify and/or define any number of subcategories of cells. In some embodiments, an average, mean, median, the distribution, or other statistical measure of senescence score of cells within a sample is recorded to determine a response to a therapeutic agent, make a diagnosis, or determine other phenotypes of a tissue or sample.

In some embodiments, the SnCs may be identified in different contexts such as different cell culture contexts, in adhered culture, in primary cells, in histology samples, in tissue slices, in 3D cultures, in organoids, and in explant cultures. In some embodiments, a transgene may be used in an animal model or a plasmid may be used in cultured cells such that the cells in either case express a fluorescent protein or label that localizes to the nucleus for the purposes of imaging live cells. Fluorescent proteins or labels that can be used may include but are not limited to histones tagged with fluorescent proteins including H2B-GFP or H2B-RFP.

Identified SnCs cells can be different cell types including but not limited to stem cells, muscle stem cells, mesenchymal stem cells, mesenchymal stromal cells, fibroadipogenic progenitors, endothelial cells, skeletal muscle cells, cartilage cells, chondrocytes, immune cells, myoblasts, adipocytes, preadipocytes, epithelial cells, and hepatocytes.

In one embodiment, other cellular data may be collected along with nuclear morphometric properties using techniques including immunofluorescence microscopy, in situ hybridization, confocal microscopy, two-photon microscopy, brightfield microscopy, any type of cell imaging, flow cytometry, modifying cells with transgenes or plasmids, or using other stains or dyes. Other cellular data that may be collected along with nuclear morphometric profiling. machine learning, and identifying SnCs can be cellular data indicative of gene expression, protein expression, protein post-translational modifications, cell morphology, cell metabolism, cell fate or cell viability. For example, the cellular data collected can be p53 expression or localization, post-translational mTOR pathway activity, actin stress fiber formation, nuclear blebbing, nuclear flattening, lamin B1 expression or localization, lamin B1 post-transitional modifications, methylated DNA, highly methylated DNA, and/or expression of cell cycle arrest regulators such as E2F.

In some embodiments, SnCs are identified in a cell line including but not limited to myoblast cell line C2C12, 3t3s, 3t3-L1s, 10t1/2 cells, mouse embryonic fibroblasts (MEFs), HUVECS, HEK 293s, or CHO cells. In some embodiments, SnCs are identified in primary cells derived from tissues including but not limited to muscle tissue, bone tissue, blood, a tumor, kidney tissue, adipose, liver, lung, brain, or heart. In some embodiments, SnCs are identified in a clinical sample, a patient sample, or a human sample. The clinical or patient sample can be primary cells, a histological sample, or a tissue slice.

In some embodiments, cells are sorted by flow cytometry before gathering nuclear morphometric properties. Cells can be sorted by the expression of a gene or surface ligand. Cells can be sorted to isolate a cell type or any other cell phenotype of interest.

In some embodiments, a nucleic acid marker may be used to enhance a signal for gathering nuclear morphometric properties of cells. A nucleic acid marker may comprise any molecule that localizes to nucleic acids, or specifically DNA, that can be imaged. Exemplary nucleic acid markers may comprise a stain, dye, or fluorescent molecule known in the art that localizes to or binds to nucleic acids or specifically DNA. In some embodiments, a nucleic acid marker may be expressed by the cell that is to be imaged, for example a fluorescent protein that localizes to DNA may be expressed by the cell. In some embodiments, a nuclear or DNA stain may be used to enhance a signal for gathering nuclear morphometric properties of cells. In some embodiments, the cells are fixed and optionally permeabilized, where fixed cells are stained with a nuclear stain or DNA marker including but not limited to DAPI or Hoechst. However, in the case of live cells, nuclear or DNA stain 141b may include stains suitable for live cells. In some embodiments, a stain 141b may be used on histological samples including tissue slices to gather nuclear morphometric properties including DAPI, hematoxylin, eosin, or Fast red nuclear stain. In some embodiments, animals or cells are engineered to express a fluorescent protein including but not limited to a fluorescent histone, that optically enhances the appearance of the nucleus, chromatin, or DNA making it easier to gather nuclear morphometric properties. For example, the fluorescent histone can be H2B-GFP or H2B-RFP.

Referring now to FIG. 1L, shown are exemplary nuclear morphometric properties 141a-d that may relate to nuclear geometry and nucleic acid organization. Nuclear morphometrics properties 141a-d may be used to identify SnCs and may be indicative of or correlated with heterochromatin, euchromatin, methylated DNA, cell flattening, nuclear flattening, cell cycle phase, DNA damage, cell type, blebbing, nuclear blebbing, cell rounding, cell attachment, cell viability and the like. For example, the nuclear morphometric properties 141a-d used may be dense foci, nuclear area, the intensity of a nuclear or DNA stain (e.g., DAPI), and nuclear circularity. However, FIG. 1L also includes whole cell morphometric properties such as cellular geometries 141e or non-geometric properties such as protein or gene expression 141f.

In various embodiments, nuclear area 141a may be defined by the number of pixels that composes a binary object representing a nucleus. In some embodiments, nuclear area is a basic quantity for measuring binary object size using the binary object or binary image to quantify area within the binary object measured by pixels then scaled against magnification of the image or object to measure in defined units (SI). In various embodiments, the intensity of the nuclear or DNA stain 141b (e.g., intensity of DAPI) may include a mean intensity, a mean pixel intensity, the arithmetic mean of each pixel value within the binary object, a median intensity, or a median pixel intensity.

In various embodiments, nuclear circularity 141c may include the formula of

$$4 π * ( \frac{( nuclear area )}{{( nuclear perimeter )}^{2}} ) ,$$

an aspect ratio, an inverse of aspect ratio, a perimeter to area ratio, or any other circularity measure. In some embodiments, a perfect circle has a circularity value equal to 1.

The number of dense foci 141d may be determined with the aid of a nuclear or DNA stain, for example the same or similar nuclear or DNA stain used to determine the intensity of the nuclear or DNA stain 141b. In various embodiments, the number of dense foci 141d may be determined by counting bright circular objects with similar sizes. Dense foci 141d may be determined by any method including a spot function and may be identified in acquired cell images by characteristics including size, contrast, and/or intensity. In some embodiments, dense foci 141d are indicative of heterochromatin. For example, the number dense foci 141d may relate to locations comprising heterochromatin. In some embodiments, the number of dense foci 141d are determined by any method known in the art for determining a number of heterochromatic foci or heterochromatin foci in a cell or specifically cell nucleus. In some embodiments, dense foci 141d may not necessarily relate to locations comprising heterochromatin. For example, dense foci 141d may simply relate to dense foci of a nucleic acid marker within a cell or specifically cell nucleus.

In various embodiments, there may be whole cell geometric properties or non-geometric properties. In the case of the cellular geometries 141e, this may include cell flattening or total cell area. In the case of protein or gene expression 141f, this may include immunofluorescence microscopy or in situ hybridization data and the like.

Referring now to FIG. 1M, shown is an exemplary method 140 for profiling and identifying SnCs. In some aspects, the method 140 may begin by identifying SnCs in step 141, using for example morphometric properties extracted from images of cells as disclosed herein. The disclosed methods may, for example, assign a senescence score to one or more cells, and all cells with a senescence score above a threshold value may be identified as SnCs. Subcategories of SnCs and/or non-SnCs may also be identified as part of this method. Additional properties of the SnCs, including but not limited to gene expression, protein content, regenerative capacity, secreted ligand profile, secreted cytokine profile, cell signaling, motility, response to treatments, or other cellular properties can be profiled in step 143. Data gathered from identified SnCs can be used to understand the biology of SnCs. Data can be used to identify a marker or biomarker of senescence including a cell surface ligand, a promoter, a gene, an expressed gene, a mutation, a protein, or post-translational modification of a protein. Data can be used to identify therapeutics or other molecules that would specifically target senescent cells.

In some embodiments, senescent cells or senescent cell load of a tissue is determined by identifying senescent cells or assigning senescence scores to cells in cell samples derived from a tissue or tissue sections. The number of senescent cells or senescent cell load of a tissue may refer to the biological age of the tissue. In other words, the senescent cell load of a tissue can be a biomarker for biological age. The biological age of a tissue can be considered to be different than chronological age.

Identified SnCs can be screened in culture for their response to therapeutic agents. The therapeutic agent can be a small molecule, a peptide, a protein, a nucleic acid, a senolytic, a chemotherapy, or any other therapeutic agent. In some embodiments, a cell culture, a primary cell sample optionally from a human patient, or a cell line is exposed to different therapeutic agents as part of a screen, and the number or percentage of identified SnCs or the distribution of cell senescence scores is subsequently recorded. In some embodiments, the number or percent of identified SnCs in a sample or the distribution of cell senescence scores is recorded in a control sample and in samples exposed to a therapeutic agent. In some embodiments, in a sample or culture derived from a patient, the number or percent of identified SnCs after exposure to a therapeutic agent is used to guide the therapy of a patient. In some embodiments, the number or percent of identified SnCs in a sample or culture derived from a patient is used to make a diagnosis. In some embodiments, cells identified as transitioning to senescence, pre-SnCs, cells with any specific range of senescence score, or other subcategories of SnCs are used in a screen of therapeutic agents. In some embodiments, the number or percent of cells in a sample identified as transitioning to senescence, pre-SnCs, or other subcategories of SnCs is recorded in a screen of therapeutic agents or to make a diagnosis. In some embodiments, the distribution of senescence scores of cells in a sample is recorded in a screen of therapeutic agents or to make a diagnosis. In some embodiments, the therapeutic agents used as part of a screen are chemotherapeutics, senolytics, known senolytics, and/or next-generation senolytics. Screens of any kind of senolytic or other therapeutic can include screening its effectiveness against any identified subgroup of SnCs.

In some aspects, a therapeutic is derived from identified SnCs. For example, secreted factors in the culture of identified SnCs, identified non-SnCs, cells identified as transitioning to senescence, or cells identified as being at any point along a senescence gradient may be used as a therapeutic. Identified SnCs, identified non-SnCs, cells identified as transitioning to senescence, or cells identified as being at any point along a senescence gradient may be used as a cell therapy optionally to aid regeneration, wound healing, or to increase or decrease inflammation and other immune cell activity. In some embodiments, large cells are sorted to enrich for SnCs.

In some aspects, the nuclear morphometric pipeline broadly described above may relate to disease models, wound healing models, tumor models, tissue regeneration models, aging models, or other models of biological processes and tissues that include identified SnCs, cells at any point along a senescence gradient, or a culture with cells of any distribution of cell senescence scores. In some embodiments, the models may comprise a monoculture, a co-culture, a 3D culture, an organoid, or an engineered tissue. In some embodiments, identified SnCs or other identified cell senescence phenotypes can be included at different ratios among other cells in the model. In some embodiments, the effects of the identified SnCs on model phenotypes are recorded. For example, identified SnCs can aid or inhibit the development of an organoid or an engineered tissue or the viability or susceptibility to treatment of cells in a tumor model. In another example, identified SnCs are incorporated in a disease model to screen therapeutic agents. In some embodiments, large cells can be sorted to enrich for SnCs.

### Computing System

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

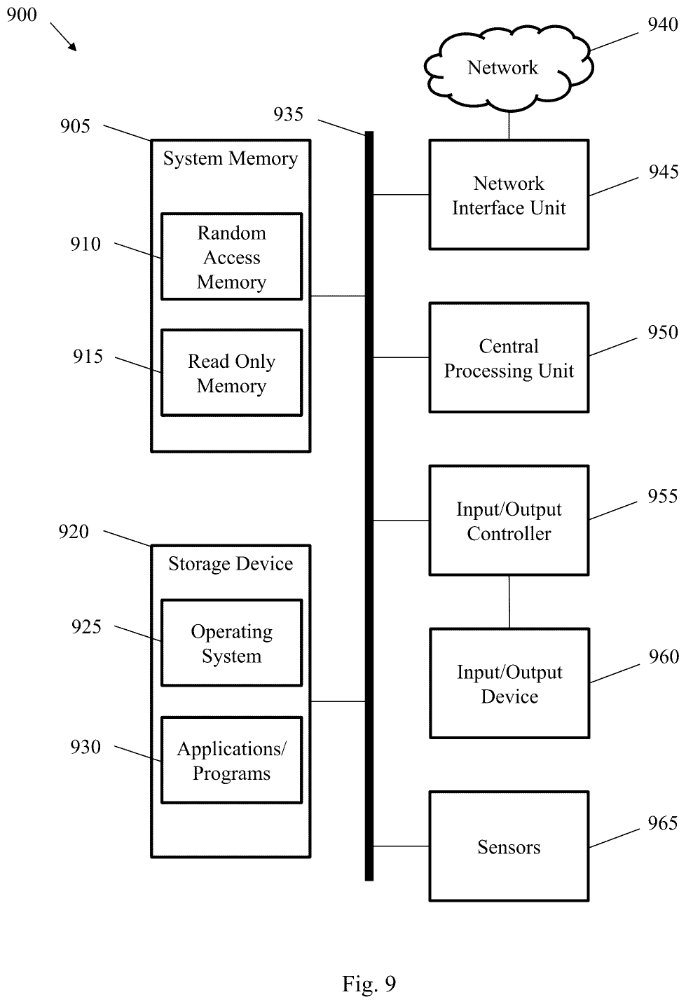

FIG. 9 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 9 depicts an illustrative computer architecture for a computer 900 for practicing the various embodiments of the invention. The computer architecture shown in FIG. 9 illustrates a conventional personal computer, including a central processing unit 950 (“CPU”), a system memory 905, including a random-access memory 910 (“RAM”) and a read-only memory (“ROM”) 915, and a system bus 935 that couples the system memory 905 to the CPU 950. A basic input/output system containing the basic routines that help to transfer information between elements within the computer, such as during startup, is stored in the ROM 915. The computer 900 further includes a storage device 920 for storing an operating system 925, application/program 930, and data.

The storage device 920 is connected to the CPU 950 through a storage controller (not shown) connected to the bus 935. The storage device 920 and its associated computer-readable media, provide non-volatile storage for the computer 900. Although the description of computer-readable media contained herein refers to a storage device, such as a hard disk or CD-ROM drive, it should be appreciated by those skilled in the art that computer-readable media can be any available media that can be accessed by the computer 900.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 900 may operate in a networked environment using logical connections to remote computers through a network 940, such as TCP/IP network such as the Internet or an intranet. The computer 900 may connect to the network 940 through a network interface unit 945 connected to the bus 935. It should be appreciated that the network interface unit 945 may also be utilized to connect to other types of networks and remote computer systems.

The computer 900 may also include an input/output controller 955 for receiving and processing input from a number of input/output devices 960, including a keyboard, a mouse, a touchscreen, a camera, a microphone, a controller, a joystick, or other type of input device. Similarly, the input/output controller 955 may provide output to a display screen, a printer, a speaker, or other type of output device. The computer 900 can connect to the input/output device 960 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 920 and RAM 910 of the computer 900, including an operating system 925 suitable for controlling the operation of a networked computer. The storage device 920 and RAM 910 may also store one or more applications/programs 930. In particular, the storage device 920 and RAM 910 may store an application/program 930 for providing a variety of functionalities to a user. For instance, the application/program 930 may comprise many types of programs such as a word processing application, a spreadsheet application, a desktop publishing application, a database application, a gaming application, internet browsing application, electronic mail application, messaging application, and the like. According to an embodiment of the present invention, the application/program 930 comprises a multiple functionality software application for providing word processing functionality, slide presentation functionality, spreadsheet functionality, database functionality and the like.

The computer 900 in some embodiments can include a variety of sensors 965 for monitoring the environment surrounding and the environment internal to the computer 900. These sensors 965 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention and are not to be construed as limiting in any way the remainder of the disclosure.

### Example #1

### Animal Models

All mice were on a C57BL/6J background and acquired from the colony at the National Institute on Aging of the National Institutes of Health. Ages ranged from 3-6 months for young, 23-25 months for aged, and 28-31 months for geriatric mice. All mice were male.

### Cell Culture and Staining

C2C12 cells were cultured according to ATCC growth media (C2GM, 10% FBS (Omega Scientific), 1× Penicillin/Streptomycin (Gibco, 15140122), and DMEM (Corning, MT10013CV)). C2C12s were used between passage 5 and 25. To induce senescence, C2C12 cells were plated between 1666-5000 cells/cm² on day 1. On Day 2, cells were treated with 300 or 500 μM H2O2 (EMD Millipore, 386790-100 ML) for 2 hours, treatment was removed, and fresh C2GM was added to cells that were cultured for an additional 3 days and then fixed with 4% PFA. For ABT-263 treatment, the same protocol was followed, and on Day 5, cells were treated with a dose-response of ABT-263 (TargetMol, T2101) for 24 hours before fixing.

Senescence was detected with the Senescence β-Galactosidase Staining Kit (Cell Signaling Technology, 9860S) according to the manufacturer's protocol with pH adjusted to 6.0. Images were captured on the Keyence BZ-X810 microscope, overlaid on DAPI staining, and substrate-positive (blue) cells were quantified.

For immunostaining, cells were fixed with 4% paraformaldehyde (PFA) for 10 minutes, rinsed in 1×PBS, permeabilized with 0.1% TritonX (Fisher Bioscientific, BP151-100) for 30 minutes, and blocked with 1% BSA (GeminiBio, 700-109P)/10% DS (Equitech Bio Inc, NC0629457)/1×PBS/0.1% tween (Fisher Scientific, BP337-100) for 1.5 hours. Following this, the cells were incubated with primary antibodies and diluent (1% BSA/10% DS/1×PBS) for 18-20 hours. Antibodies included: γH2A.X (Cell Signaling Technology, 9718), Ki67 (Abcam, ab 15580), Cleaved-Caspase-3 (Cell Signaling Technology, 9661). After washing with 1×PBS to remove the primary antibodies, cells were incubated with diluent and secondary antibodies for 2 hours. Secondary antibodies included: Alexa Flour 555 Donkey anti-Rabbit (Thermo Scientific, A-31572). Cells were then washed with 1×PBS and counterstained with DAPI (Invitrogen, D1306). All samples were maintained at 4° C. until imaging with the Nikon Eclipse Ti2-LAPP Inverted Microscope System. Images were taken at 20× with 1 to 5 images taken per well. Images were then quantified with NIS-Elements AR Analysis Software-Nikon Instruments for positivity of fluorescence and/or intensities.

### Knee Joint Processing for Cartilage Histology

Samples were fixed in 10% buffered formalin for 72 hours and decalcified in 10% formic acid for 10 days. Decalcified samples were embedded in paraffin, and 4 μm sections were cut perpendicular to the cartilage surface. Sections were stained with Safranin O/Fast green (Electron Microscopy Sciences, 477-73-6/Sigma-Aldrich, 2353-45-9) using routine methods. Cartilage destruction in the mice was examined using Safranin O staining.

### RT-qPCR

Total RNA extraction from cultured cells was performed according to the manufacturer's protocol with the RNeasy Micro Kit (Qiagen, 74004). RNA quantitation was assessed on the Thermo Scientific NanoDrop One, and cDNA was generated with the RevertAid RT Reverse Transcription Kit (Thermo Scientific, K1691). The mRNA expression was determined on a QuantStudio5 Real-Time PCR System using FastStart SYBR Green Master (Roche, 04673484001). Relative expression levels were calculated with the 2^{-ΔΔCT} method using GAPDH as the reference gene. Primers (Thermo Scientific) sequences are in Table 1 and were used at 0.2 uM concentration.

### Muscle Injury

Mice were anesthetized with isoflurane, and injury was induced with 90 ul of 1.2% BaCl2 (Sigma-Aldrich, B0750) injected intramuscularly into the lower hindlimb muscles, which were isolated at the days post injury (DPI) noted in the main text. The gastrocnemius was used to isolate cells from injured SkM.

### Cell Isolation and Fluorescence Activated Cell Sorting

To isolate cells from muscle tissue, dissected tissue was minced with scissors and then incubated in 760 U/mL collagenase type 2 (Worthington Biochemical, LS004177) in Ham's F10 (Cytiva, SH30025.01) for ˜1 hour in a 37° C. shaking water bath, then washed with wash media (WM, 10% Horse Serum (HS) (Thermo Scientific, 16050122) in Ham's F-10), and digested again in a 1 to 8.5 dilution of both 1000 U/ml collagenase type 2 (Worthington Biochemical, LS004177) and 11 U/ml dispase (Thermo Scientific, 17105041) in WM for 30 minutes in a 37° C. shaking water bath. The samples were triturated seven times with a syringe fixed with a 21-gauge needle and then passed through a 40 μm strainer (Falcon, 352340). The single-cell suspension was stained with fluorescent-conjugated antibodies for 30 minutes at 4° C. Fluorescent-conjugated antibodies included CD-45 (BioLegend, 103101), Sca-1 (Biolegend, 108120), VCAM (Biolegend, 105720), CD-31 (Biolegend, 102510). Cells were then passed through blue-top filters (Falcon, 352235) and sorted using the Sony MA900 Multi-Application Cell Sorter Software (Version 3.3.0) for endothelial cells (C31+), immune cells (CD45+), FAPs (Sca-1+/CD31−/CD45−), and SCs (VCAM1+/CD31−/CD45−/Sca-1−). Cells were plated in ECM-coated (Sigma Aldrich, E6909) 96-well plates containing WM, centrifuged at 500 RFC for two minutes, and incubated overnight to allow for adherence. After 12 hours, cells were fixed with 4% PFA for 10 minutes and washed with 1×PBS (Gibco, 14-200-075). The cells were then stored at 4° C. until imaging.

For EdU staining, cells were sorted using the Sony MA900, plated on ECM-coated 96-well plates containing growth media ((GM), 10% HS, 20% FBS, 1:100 Penicillin/Streptomycin, and 2.5 ng/ml FGF (PeproTech, 100-18B) in Ham's F-10), then incubated for four (SCs) or five (FAPs) days with a pulse of EdU (5 μM) five hours prior to fixing with 4% PFA for 10 minutes and washed with 1×PBS. EdU incorporation was detected with the Alexa Fluor 555 Click-iT™ Plus EdU Cell Proliferation Kit (Thermo Scientific, C10638) using the manufacturer's recommended protocol and stored at 4° C. until imaging.

To isolate chondrocytes from the knee joints, dissected cartilage was collected using a scalpel as previously described, except for minimizing endochondral bone carryover (Lee, K.-A. et al. Front. Aging 3, 900028 (2022)). The cartilage was then incubated in 760 U/mL collagenase type 2 in Ham's for about 2 hours at 37° C. shaking water bath. The samples were then passed through a 40 μm filter (Falcon, 352340). The single-cell suspension was stained with fluorescent-conjugated antibodies for 15 minutes at 4° C. Fluorescent antibodies included Terr-119 (Invitrogen, 48-5921-82) and CD-45 (BioLegend, 103101). Cells were then stained with Propidium Iodide (Invitrogen, P3566), passed through blue-top filters, and sorted using the Sony MA900 Multi-Application Cell Sorter. Cells were plated on ECM-coated 96-well plates in WM, centrifuged at 500 RFC for 2 minutes, incubated overnight, and fixed 12 hours later with 4% PFA for 10 minutes. All samples were maintained at 4° C. until imaging. To detect senescence-associated β-galactosidase, the FastCellular Senescence Detection Kit (MP Biomedical, 092690301) was used prior to sorting according to the manufacturer's protocol. Briefly, samples were incubated at 37° C. in Bafilomycin A1 for 1 hour, stained for 30 minutes at 37° C. with SPIDER-β-gal/Bafilomycin A1, resuspended in WM and maintained at 4° C. until analyzed on the Sony MA900.

### Fluorescence Imaging

Fluorescent images were acquired using the Nikon Eclipse Ti2-LAPP Inverted Microscope System equipped with the NIS-Elements Advanced Research acquisition software (Version 5.42.03). Three representative high-magnification images were acquired with a 20× objective per well. For EdU-stained samples, the entire well was imaged. z-stacked Images were taken in 1 μm increments.

### Pre-Processing Pipeline

Image pre-processing was performed with NIS-Elements Advanced Research (AR) Analysis Software (Nikon Instruments, Version 5.42.03). Initial preprocessing started with a 3D deconvolution to remove noise from a z-stacked image. Then, a max intensity projection was used to allow for all fluorescence to appear on a single plane and ease analysis of image. To further refine the images, a low pass filter was used to pass only details larger than a set pixel value and remove small irregularities. Finally, a rolling ball function was used to further differentiate background and fluorescence intensities.

### Nuclei Phenotypic Extraction:

Image analysis was performed with the NIS-Elements AR Analysis Software to achieve single nuclei resolution. To detect nuclei, a threshold function was used on images of wells stained with DAPI, allowing the formation of binary objects in the image. Limitations were made on size and circularity to remove objects such as debris or overlapping nuclei. By creating the binary image, nuclei could be separated, counted, and measured for size, mean intensity, and circularity. To detect foci, a spot function was used to count the number of bright circular objects that contrasted from detection surrounding pixels. Limitations on spots included size, contrast, and intensity. These spots were then aggregated to only count bright spots that were present in the binary data of the detected nuclei. For C2C12 cells, as well as primary FAPs and SCs, analyses were automated through the software on whole images. For primary endothelial and immune cells, analyses were done both on whole images and manually to avoid misidentification due to cell aggregation.

### UMAP Presentation and Clustering

Using R studio (Version 4.3.1), the four phenotypic measures of nuclei were imported and normalized using a z-score within their respective parameters. Using the R studio package UMAP (Version 0.2.10.0), a two-dimensional visualization (n_neighbors=20, n_components=2) of the four parameters was made to localize visually similar cells. To form the senescence score, the prior step was repeated with n_components=1 and required a high number of cells to achieve optimal dynamic range. For three-dimensional visualization, the prior step was repeated with n_components=3. To reduce nuclei number for visual clarity, a set number of cells were plotted after being selected through a random number generator. To cluster visually similar cells, package “factoextra” (Version 1.0.7) was used, specifically the function “fviz_cluster” which applies a k-means algorithm on the normalized phenotypic measures. Data was exported and graphed with GraphPad Prism.

### Statistical Analysis and Graphical Representation

GraphPad Prism (Version 9.5.0) was used for all statistical analyses and error bars represent the mean±standard error of the mean (SEM). All tests were conducted using a two-sided T-tests, unless otherwise indicated, with stars indicating the following: NS P≥0.05, * P≤0.05, ** P≤0.01, *** P≤0.001, **** P≤0.0001.

### Figure Construction

All figures were arranged using BioRender. All schematics were made in BioRender. Graphs, unless otherwise stated, were made using GraphPad Prism (Version 9.5.0). FIG. 2A was made using the Rstudio package ggplot2 (version 3.3.4) and extension ggally (Version 2.2.1). FIG. 2C was made using the Rstudio package kohonen: supervised and unsupervised self-organizing maps (Version 3.0.12). Three-dimensional UMAPs were made using the Rstudio package plotly (Version 4.10.4). FACS plots were constructed using FlowJo (Version 9).

### Results

To study the nuclear phenotypic changes associated with senescence, an in vitro culture system was designed to induce senescence in a myoblast cell line (C2C12) using hydrogen peroxide (H2O2) to stimulate oxidative stress and damage (FIG. 1A) (Yagi, M. et al. Sci. Rep. 13, 7697 (2023)). The treatment led to a decrease in cells expressing the proliferation marker Ki67+, an increase in the expression of the DNA damage marker γH2AX+, and an increase in SA-β-gal activity, all in a dose-dependent manner (FIG. 1B, C). The lack of Caspase-3 staining confirmed cells were not merely entering apoptosis (FIG. 1B). In addition, real-time quantitative PCR (qPCR) confirmed a SASP profile in treated cells (FIG. 1D). Consequently, an H₂O₂ treatment was established that induced bona fide senescence and thus a robust system to study related nuclear morphological changes.

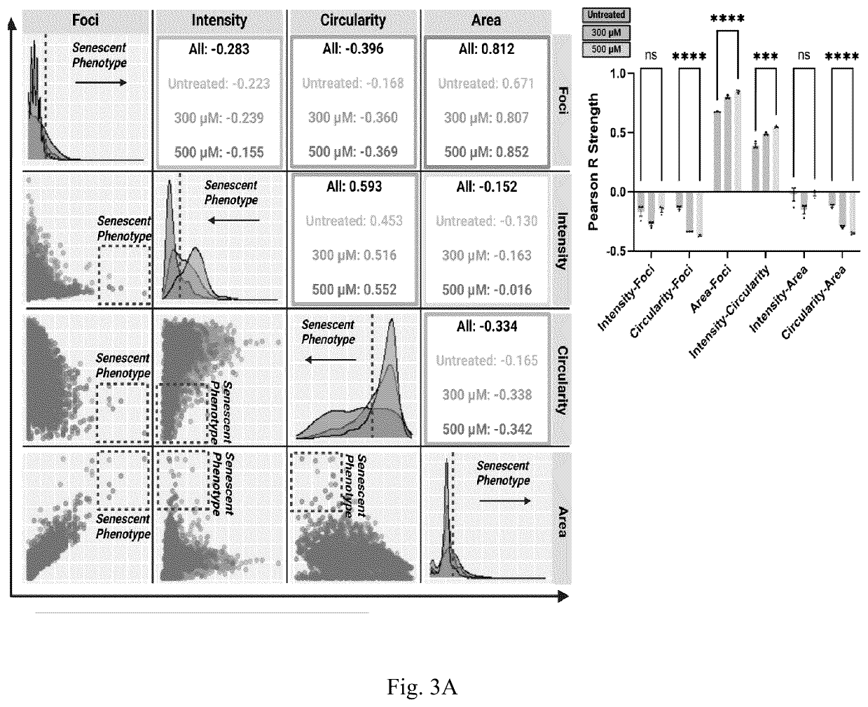

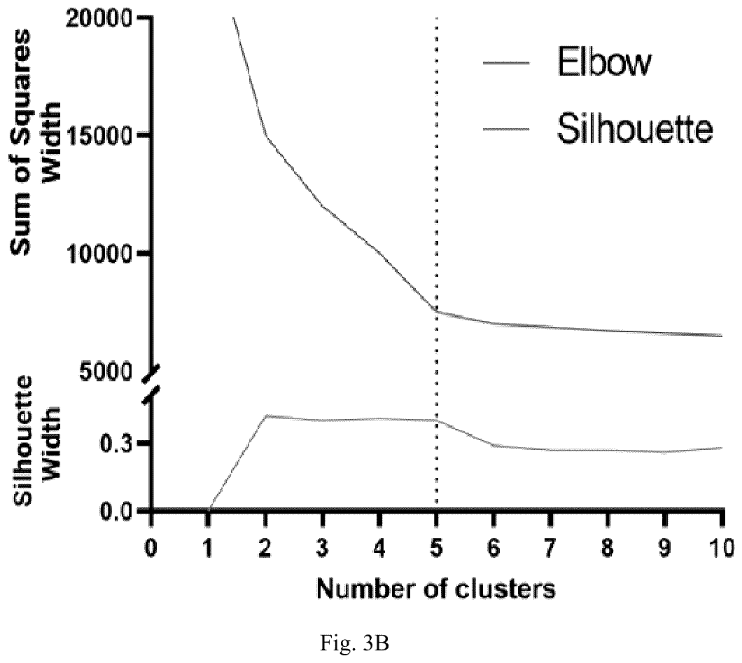

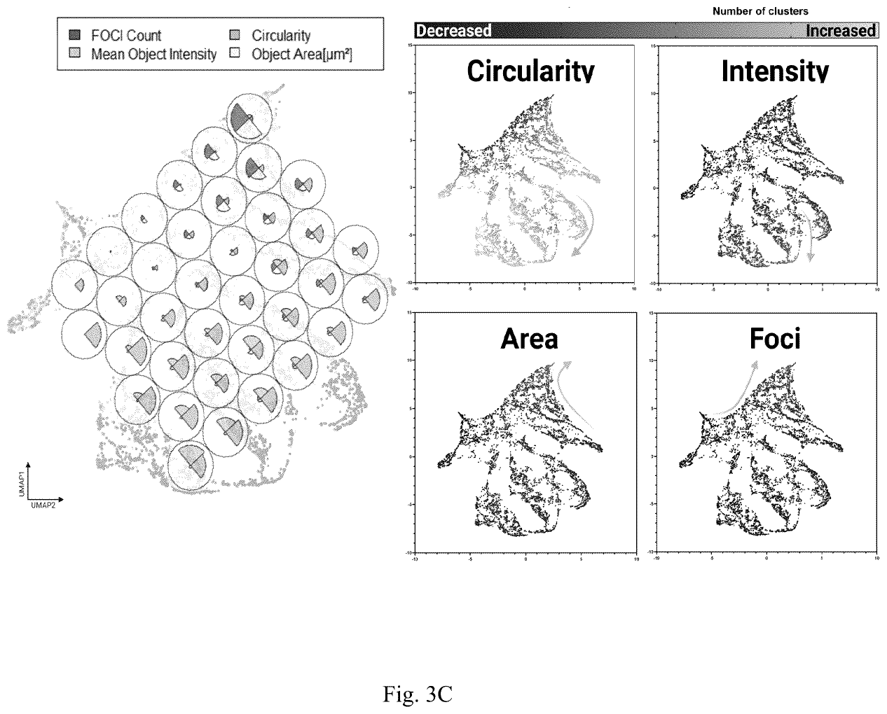

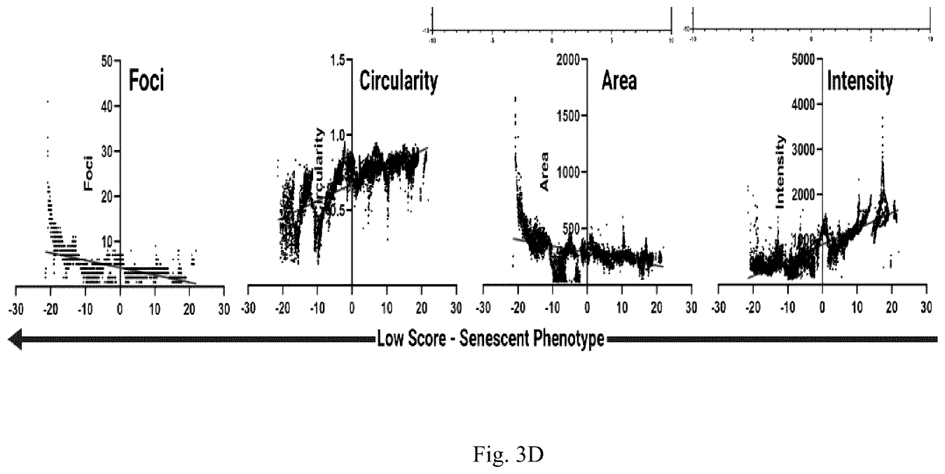

The nuclear phenotypes were then examined associated with increasing H₂O₂ treatment by staining with DAPI to provide an enhanced definition of nuclei. With increasing H₂O₂, an increase was detected in nuclear size and dense foci, and a concomitant decrease in nuclear circularity and DAPI intensity (FIG. 1E), characteristics that have each independently been suggested associated with senescence (Freyter, B. M. et al. Cells 11, 273 (2022), Zhang, R. et al. Dev. Cell 8, 19-30 (2005)). The increase in dense foci may relate to an increase in heterochromatin. For example, each dense foci may relate to a location that includes heterochromatin. The correlation strength between paired parameters similarly increased with the greater concentration of H₂O₂, indicating that SnCs share many of these nuclear morphologies (FIG. 3A). To identify those cells that shared all parameters and were most indicative of senescence, these four parameters were dimensionally reduced using a Uniform Manifold Approximation Projection (UMAP) to generate a two-dimensional representation with single-cell resolution (FIG. 3C) (Chen, Ozanne, J.-H. et al. Methods of Cellular Senescence Induction Using Oxidative Stress. 179-189 (2007)). A k-means unsupervised machine learning algorithm was applied to define groups with definitive morphometric similarity and determine the senescent nuclei sharing all four parameters (FIG. 1F, FIG. 3C) (Stolarek, I., et al. iScience 25, 105142 (2022)). An elbow plot and silhouette method were both used to delineate the number of clusters (FIG. 3B). Further reduction resulted in a one-dimensional “score” to achieve single-cell resolution for direct comparisons between nuclei, which established a “senescent gradient” (FIG. 1F, FIG. 3D). Using the defined clusters of nuclei, a population with extreme morphometrics was identified and putatively labeled these as bona fide SnCs. Another population that shared many of these traits, but not all, were deemed “pre-senescent” cells (FIG. 1F). The percentage of cells identified as both senescent and pre-senescent was greatest in the highest H₂O₂ treated samples (FIG. 1F).

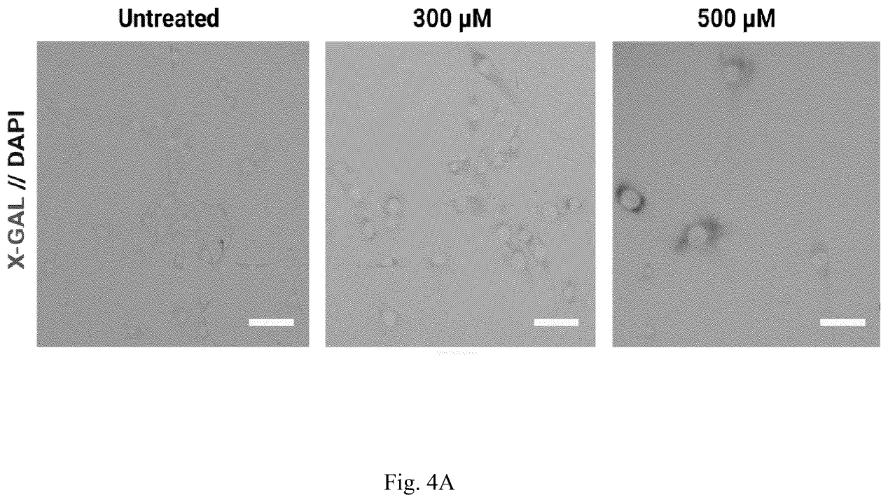

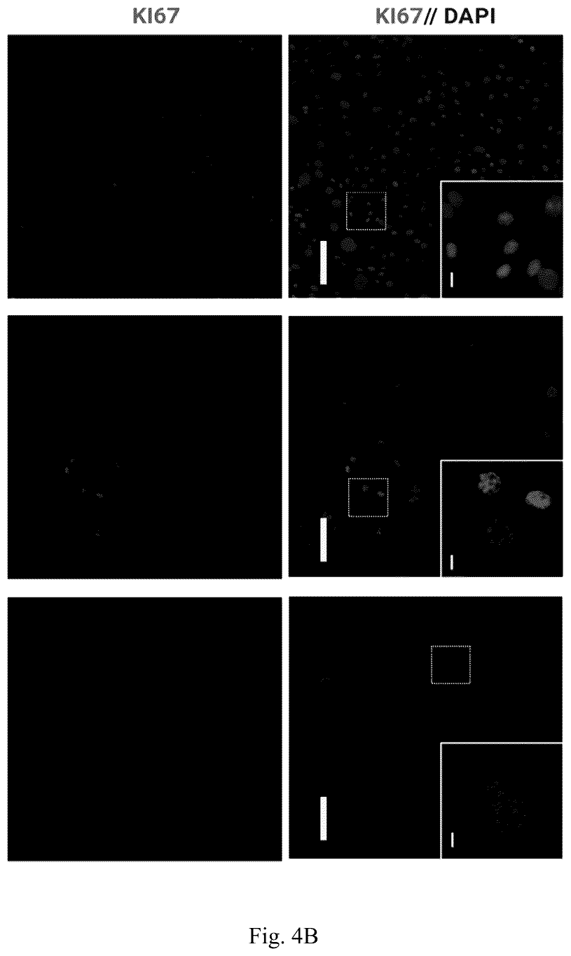

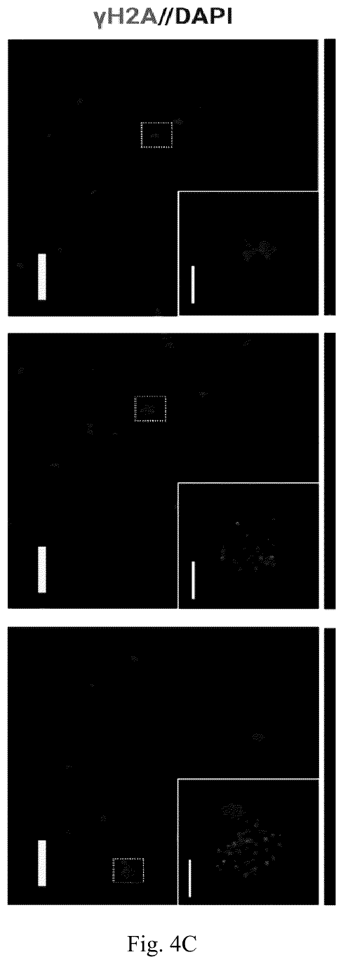

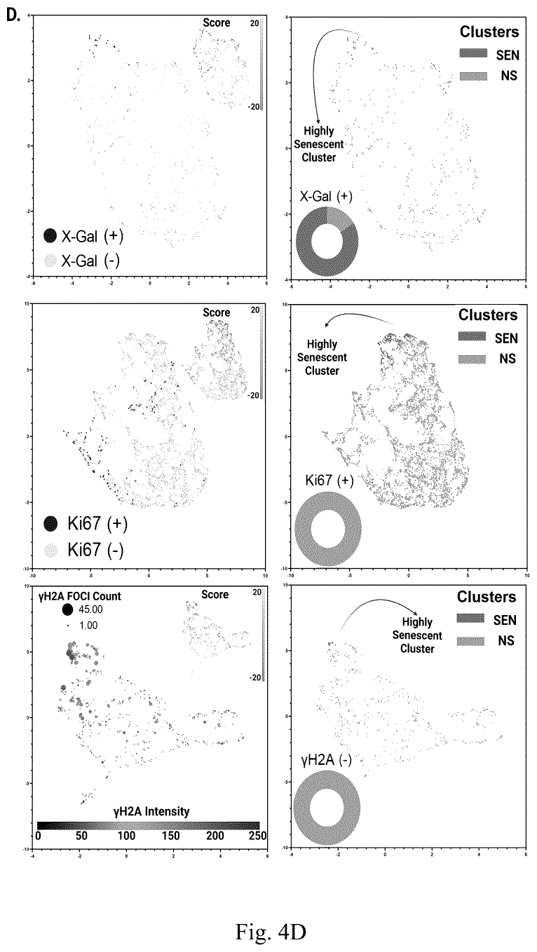

To confirm that cells clustered by the nuclear morphometric pipeline (NMP) were genuinely senescent, their cell cycle status (Ki67), degree of DNA damage (γH2AX), and SA-β-gal activity were assessed. The senescent nuclei identified by the NMP demonstrated a decrease in the Ki67 staining, an increase in γH2AX MFI, and an increase in SA-β-gal activity (FIG. 1G. FIG. 4A-D), collectively supporting the senescent phenotype. Additionally, treatment with the senolytic Navitoclax (ABT-263), resulted in a dose-dependent reduction in the cells with nuclei in the senescent cluster, with the greatest lysis in the highest H₂O₂ treated samples (FIG. 1H).

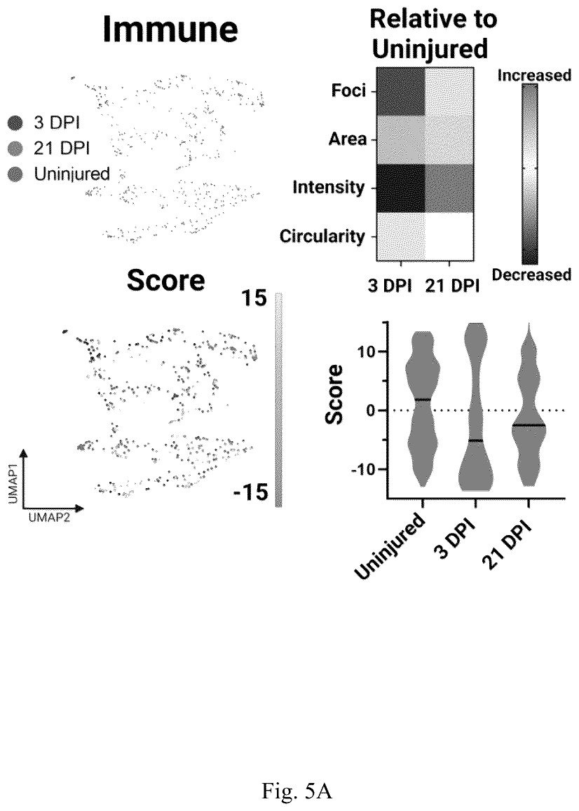

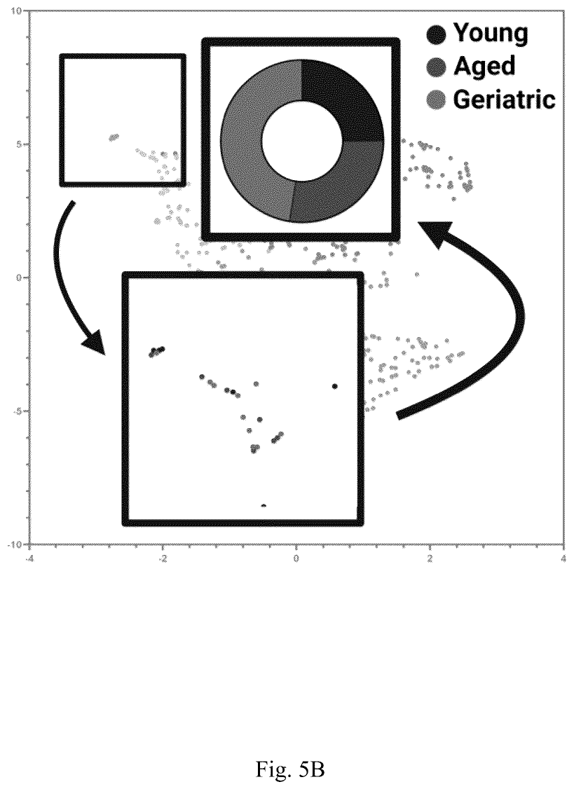

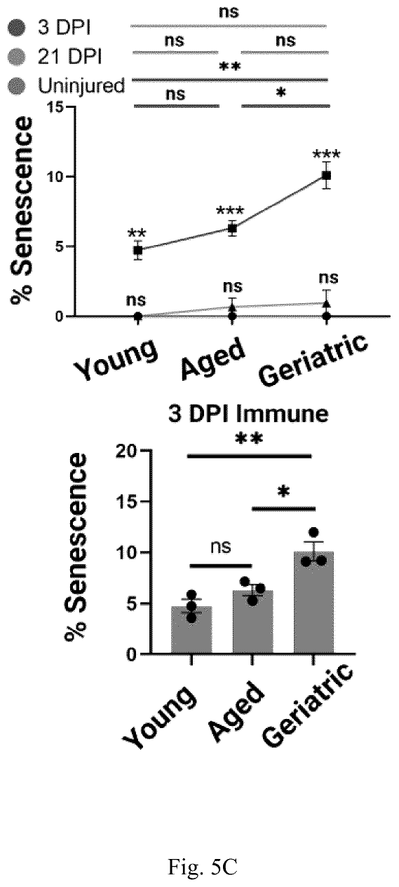

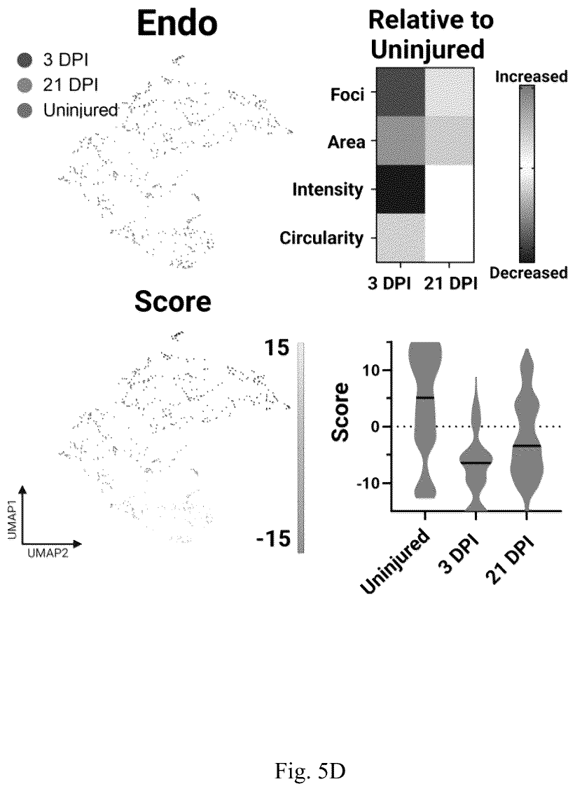

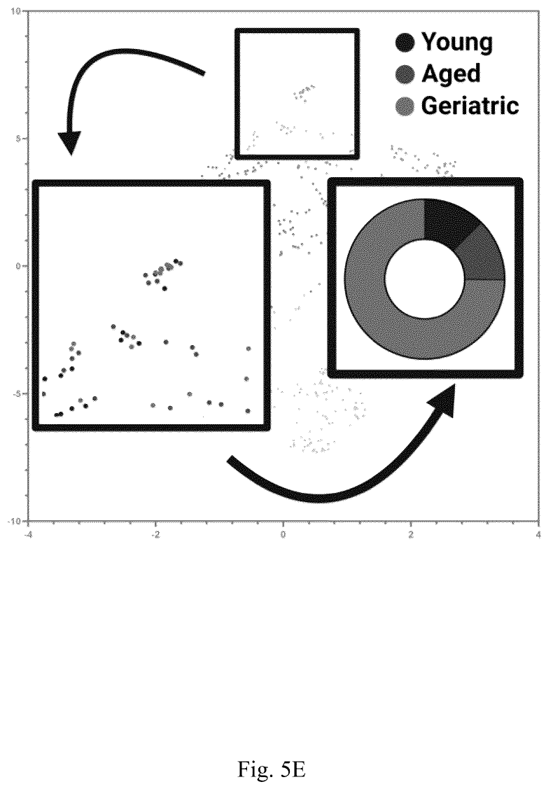

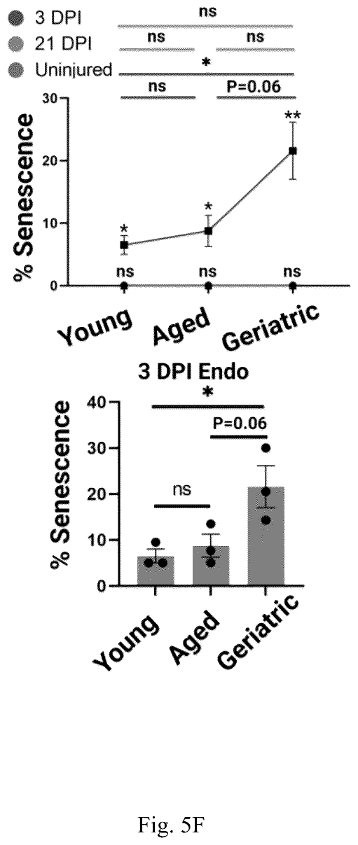

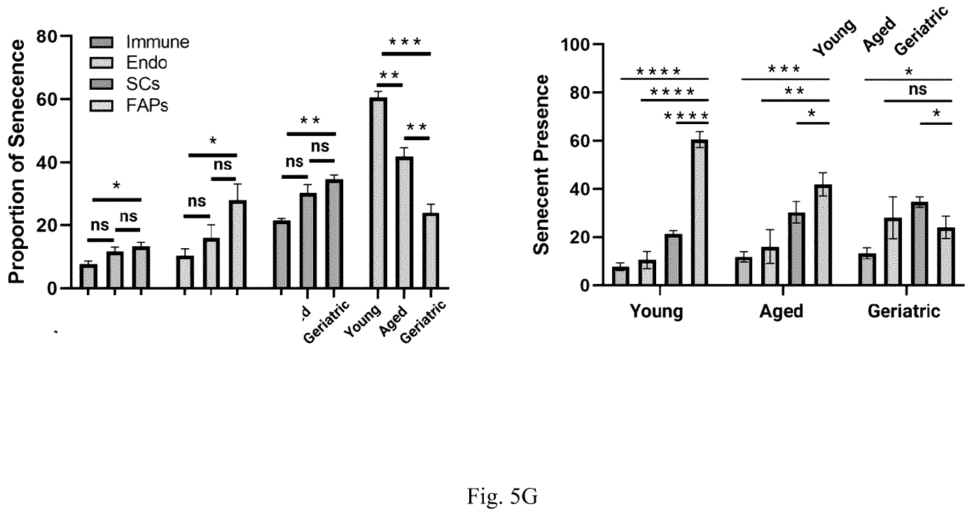

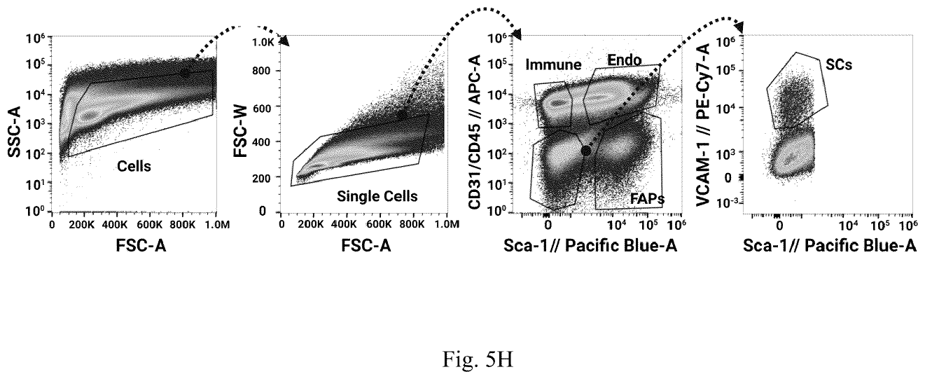

To translate the NMP to the in vivo application and identification of SnCs, homeostatic and regenerating SkM was examined, where this tissue was reported to contain SnCs in both contexts across age (FIG. 2, FIG. 5) (Duran, I. et al. Nat. Commun. 15, 1041 (2024)). To induce regeneration, BaCl₂ was injected intramuscularly into the lower hind limb muscles in young (5 months), aged (24 months), and geriatric (30 months) mice (FIG. 2A). Using the NMP, a number of cells were analyzed including: muscle stem cells (aka satellite cells—SCs), mesenchymal stem cells (MSCs, aka fibroadipogenic progenitors—FAPs), endothelial cells (ECs), and immune cells (ICs) that were isolated by FACS from uninjured and injured tissue at 3 days post-injury (DPI), a time when the mononuclear cellular response is at its greatest, and at 21 DPI when muscle regeneration is nearly complete except for continued remodeling and hypertrophy (FIG. 2, FIG. 5) (Ahmad, A. & Dey, L. Data Knowl. Eng. 63, 503-527 (2007), Wosczyna, M. N. et al. Cell Reports 27, 2029-2035.e5 (2019)).

Following the dimensional reduction of the four nuclear morphometrics for each cell population from uninjured, 3 DPI, and 21 DPI, a spectrum of non-SnCs to SnCs was clearly identified, revealing a dynamic milieu of SnCs, both across the injury time course and across the age of mice (FIG. 2, FIG. 5) In homeostatic SkM, SnCs are essentially absent regardless of age, consistent with many recent studies and the evolving paradigm around senescence in uninjured tissue (FIG. 2D, G, FIG. 5C, F) (Moiseeva, V. et al. Nature 613, 169-178 (2023)). Following injury, there is a robust increase in SnCs at 3 DPI, supporting SnCs as necessary for efficient regeneration (FIG. 2D, G, FIG. 5C, F). In young SkM, FAPs were the major fraction of SnCs, with SCs, ECs, and ICs only contributing a minority of the SnC population at 3 DPI (FIG. 2H, FIG. 5G). These data suggest that FAP senescence is a requirement for young SkM regeneration. Intriguingly, while the senescence response in aged tissue remains robust at 3 DPI, SCs become the majority of SnCs, while FAPs fall into the minority in geriatric SkM (FIG. 2H, FIG. 5G). As SCs become more senescent in geriatric tissue, this is predictably detrimental to regeneration as it limits the pool of myogenic precursors needed to recapitulate SkM (Baghdadi, M. B. & Tajbakhsh, S. Dev. Biol. 433, 200-209 (2018)).

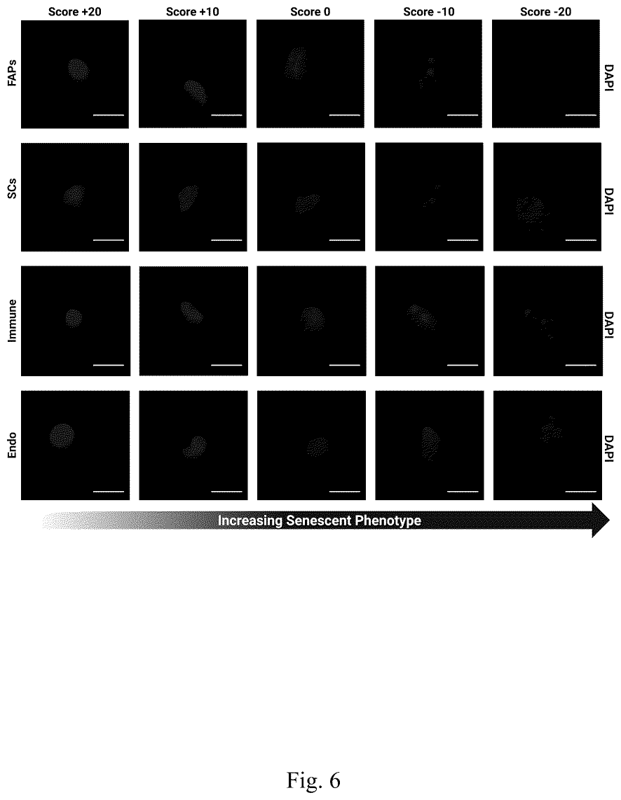

The data reveals a complex age-dependent SnC environment in early regeneration and denotes their seemingly beneficial and detrimental role in young and aged SkM, respectively (FIG. 2C, F, FIG. 5B, E). Interestingly, at 21 DPI, when muscle is largely rebuilt with regenerated myofibers and mononuclear cell numbers at homeostatic levels, SnCs have largely resolved, as demonstrated by the rebound seen in the scoring system (FIG. 2B, E, FIG. 5A, D, FIG. 6A) (Liu, L. et al. Cell Stem Cell 23, 544-556.e4 (2018)). However, a small number of SCs remain senescent in geriatric SkM, reinforcing the potential impact of senescence in the myogenic precursor population, leading to less efficient regeneration in aged SkM and chronic inflammation from continued SASP expression (FIG. 2G). Thus, the data supports the necessity of FAP senescence in young tissue for efficient regeneration, a process that is blunted in aged tissue and concomitantly demonstrates that SCs acquire a senescent phenotype during aging, which is consistent with the reported age-dependent decrease in myogenicity of these cells (Wosczyna, M. N. & Rando, T. A. Dev. cell 46, 135-143 (2018)).

Furthermore, the loss of proliferative capacity was correlated in aged cells (decreased EdU incorporation) with the NMP-identified SnCs, focusing on SCs and FAPs due to their aforementioned dynamic senescence across aging (FIG. 7A). A 10% and 30% reduction was detected in EdU in SCs and FAPs, respectively, when comparing cells from aged and young mice that were grown in culture (FIG. 7B, C). The cells identified as the SnC population by the NMP proved to have significantly reduced EdU positivity, confirming an exit from the cell cycle (FIG. 7B, C).

Finally, to test if the NMP can be applied to additional aged-related degenerative disorders associated with SnC load, the pipeline was used to evaluate chondrocytes in primary osteoarthritis in aging mice (FIG. 8A) (Zeng, W. et al. Restoration of CPEB4 prevents muscle stem cell senescence during aging. Dev. Cell 58, 1383-1398.e6 (2023)). Degenerative articular cartilage from geriatric mice was compared to its healthy counterpart from young mice (FIG. 8B). A similar approach to the above SkM process was implemented to analyze FACS-isolated cells enriched for chondrocytes and ICs (FIG. 8E). A robust senescent signature was revealed in chondrocytes from geriatric cartilage that represented about a 10-fold increase in SnCs compared to chondrocytes from young tissue (FIG. 8C). ICs from geriatric mice also showed an increase in SnCs compared to young counterparts, with ˜3-fold increase (FIG. 8D). These data support the current paradigm that chondrocytes of arthritic milieus are senescent and demonstrate the utility of the NMP as a robust means to identify SnCs from primary tissue.

TABLE 1

| GenBank | PrimerBank | Primer | Forward Sequence | Reverse Sequence |
|---|---|---|---|---|
| Accession | ID | Name | (5′-3′) | (5′-3′) |
| NM_011333 | 141803162c1 | mCCL2 | TAAAAACCTGGATCGG | GCATTAGCTTCAGATT |
| | | | AACCAAA | TACGGGT |
| | | | (SEQ ID NO: 1) | (SEQ ID NO: 2) |
| NM_001111099 | 6671726a1 | mCDKN1A | CCTGGTGATGTCCGAC | CCATGAGCGCATCGC |
| | | | CTG | AATC |
| | | | (SEQ ID NO: 3) | (SEQ ID NO: 4) |
| NM_013693 | 133892368c1 | mTNF | CAGGCGGTGCCTATGT | CGATCACCCCGAAGT |
| | | | CTC | TCAGTAG |
| | | | (SEQ ID NO: 5) | (SEQ ID NO: 6) |
| NM_001037722 | 109148516c2 | mADAM15 | ATGGCACCCGAATGGT | CTCCAGTGTATAGCCT |
| | | | CAG | CTCTCTG |
| | | | (SEQ ID NO: 7) | (SEQ ID NO: 8) |
| NM_031168 | 13624310c1 | mIL6 | CTGCAAGAGACTTCC | AGTGGTATAGACAGG |
| | | | ATCCAG | TCTGTTGG |
| | | | (SEQ ID NO: 9) | (SEQ ID NO: 10) |
| NM_001164197 | 255958311c3 | mMMP19 | CCTGGTCCCATGCCAA | CCCTTGAAAGCATAA |
| | | | ACC | GTCTTCCC |
| | | | (SEQ ID NO: 11) | (SEQ ID NO: 12) |
| NM_011577 | 6755774c1 | mTGFβ1 | CCACCTGCAAGACCAT | CTGGCGAGCCTTAGT |
| | | | CGAC | TTGGAC |
| | | | (SEQ ID NO: 13) | (SEQ ID NO: 14) |
| NM_010721 | 188219588c2 | mLMNB1 | GAGTATGAGGCGGCA | CATCTGCTAACTGCT |
| | | | CTAAAC | TTTTGGC |
| | | | (SEQ ID NO: 15) | (SEQ ID NO: 16) |
| NM_008610 | NA | mMMP2 | TGCAGGAGACAAGTT | GACGGCATCCAGGTT |
| | | | CTGGA | ATCAG |
| | | | (SEQ ID NO: 17) | (SEQ ID NO: 18) |
| NM_008084 | NA | mGAPDH | TCAAGAAGGTGGTGA | GTTGAAGTCGCAGGA |
| | | | AGCAG | GACAA |
| | | | (SEQ ID NO: 19) | (SEQ ID NO: 20) |

In addition to hydrogen peroxide, etoposide and doxorubicin is used on C2C12 and 3T3_L1 cell lines to induce senescence and then senescence is assayed with the NMP. Senescent cells in a sample are depleted by using senolytic drugs and the NMP is used to identify senescent cells.

Images of tissue sections from uninjured and injured skeletal muscle and cartilage from young, aged, and geriatric mice are obtained and the NMP is used to identify senescence. The tissue sections are stained with a nuclear marker.

The senolytic drugs ABT-263 and D+Q are administered to young and geriatric mice to modulate senescence in vivo. The NMP is used to identify senescence in samples derived from the mice.

A deep learning approach is used to identify nuclear morphometrics that are used in addition to dense foci, nuclear area, nuclear circularity, and DAPI intensity in the NMP to identify senescence. High Content Analysis (HCA) and Exploratory Factor Analysis (EFA) algorithms are used to characterize senescent and non-senescent nuclei. A Scree plot is used to determine the number of nuclear morphometrics that differ between senescent and non-senescent cells, and Lasso Regression is used to optimize the number of nuclear morphometric properties used in the NMP. UMAP is used for dimensional reduction and Gaussian and centroid clustering to group cells.

### Example #2

Cellular senescence is an irreversible state of cell cycle arrest with a complex role in tissue repair, aging, and disease. However, inconsistencies in identifying cellular senescence have led to varying conclusions about their functional significance. Disclosed herein is a machine learning-based approach that uses nuclear morphometrics to identify senescent cells at single-cell resolution. By applying unsupervised clustering and dimensional reduction techniques, the disclosed system forms a robust pipeline that distinguishes senescent cells in cultured systems, freshly isolated cell populations, and tissue sections. The disclosed method reveals dynamic, age-associated patterns of senescence in regenerating skeletal muscle and osteoarthritic articular cartilage. The disclosed approach offers a broadly applicable strategy to map and quantify senescent cell states in diverse biological contexts, providing a means to readily assess how this cell fate contributes to tissue remodeling and degeneration across lifespan.

A consequence of aging is the progressive loss of effective tissue regeneration, which has been linked to the accumulation of senescent cells (SnCs) (Tuttle, et al., Aging Cell, 2020). Cellular senescence, or the permanent exit from the cell cycle, typically results from intrinsic damage and is an evolutionary mechanism that limits the propagation of pathological and precancerous cells (Wang, L., et al., Nat. Rev. Cancer, 2022). While SnCs exit the cell cycle, they remain metabolically active, releasing inflammatory cytokines as part of the senescence-associated secretory phenotype (SASP) (Coppé, J.-P., et al., Pathol. Mech. Dis., 2010). These cytokines can stimulate the immune system to aid tissue repair, but can also cause chronic inflammation if SnCs accumulate, as in aging (Hu, L. et al., Front. Cell Dev. Biol., 2022).

Determining whether SnCs are beneficial or detrimental is likely founded in the contextual influences of the tissue. However, reports vary on whether these cells benefit or hinder similar processes in identical tissues (Rhinn, M., Development 146, 2019; and Moiseeva, V. et al. Nature, 2023). These dichotomies are due to confounding factors such as the difficulty in reliably identifying SnCs and the heterogeneity within this population. Many of the markers used to suggest a cell has senesced (e.g., p16, p21, HMGB1, and Lamin B1) demonstrate great variability, differing in assays and from study to study. A leading biomarker for SnCs known as senescence-associated-β-galactosidase (SA-β-gal) relies on their increased lysosomal density (Valieva, Y., Diagnostics, 2022). However, even this can have high variability due to its dependence on strict pH control. Complicating matters, the limited number of SnCs in tissue makes it challenging to refine assays for consistent and conclusive results. Due to the cell cycle component of senescence, altered nuclear morphologies have been suggested as an alternative means to identify SnCs, characteristics that require simple reagents to resolve and can be detected in minimal cells, thus providing consistent categorization of SnCs (Huang, W., et al., Nat. Rev. Nephrol., 2022; Freyter, B. M. et al., Cells, 2022; and Zhang, R. et al., Dev. Cell, 2005). Recently, there have been reports of using supervised learning approaches to elucidate nuclear morphologies associated with senescence (Heckenbach, I. et al., Nat. Aging, 2022; and Duran, I. et al., Nat. Commun., 2024). These approaches have shown promise in applying measures derived from training datasets for specific tissue contexts.

The disclosed system includes a robust pipeline using nuclear morphometrics and machine learning techniques to identify bona fide senescence induced in culture with alternative methods and with multiple cell types. Importantly, the system maintains an unsupervised learning approach to broaden the applicability of the system across mechanisms of senescence, cell types, and tissue contexts, where unsupervised clustering and dimensional reduction were biologically validated and interpreted within a semi-supervised framework. The nuclear morphometric pipeline (NMP) was translated to detect SnCs in young, aged, and geriatric skeletal muscle (SkM) and cartilage, two tissues reported to contain SnCs, proving its efficacy in detecting in vivo senescence (Wan, M., et al., Bone Res., 2021; Saito, Y., et al., Nat. Commun., 2020; and Yagi, M., et al., Sci. Rep., 2023). The disclosed system reveals an age-dependent dynamic milieu of bona fide SnC populations in these tissues and can be readily deployed with simple and consistent nuclear assessments.

### Results

### Oxidative Stress Induces Senescence in Myoblast Cultures

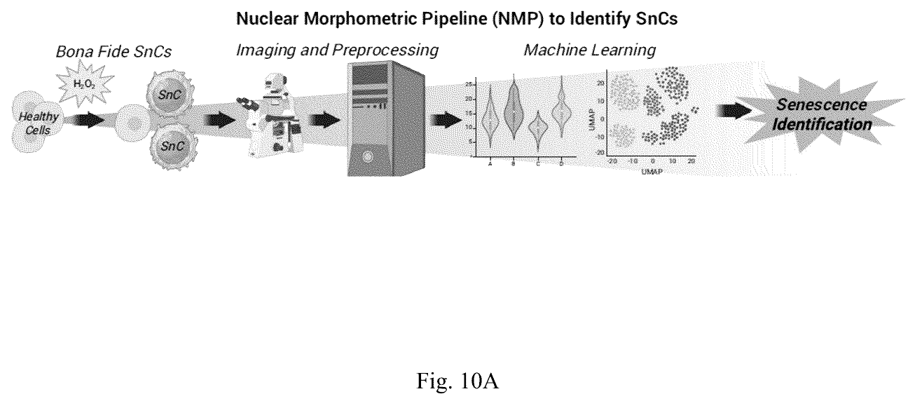

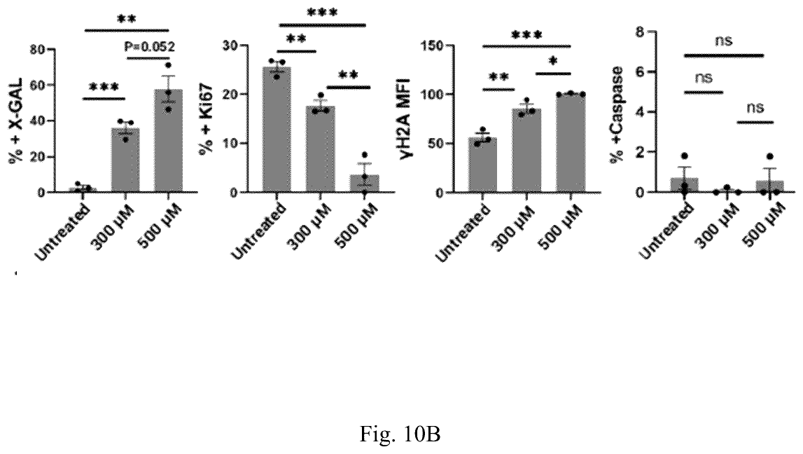

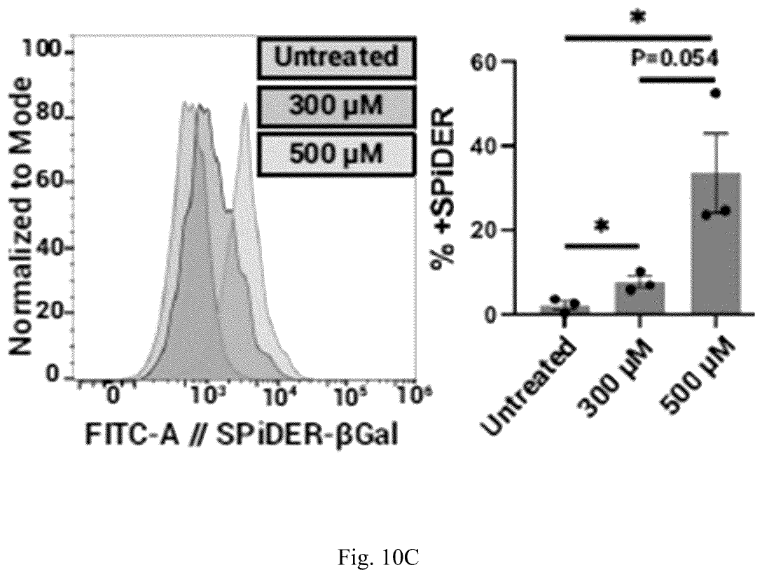

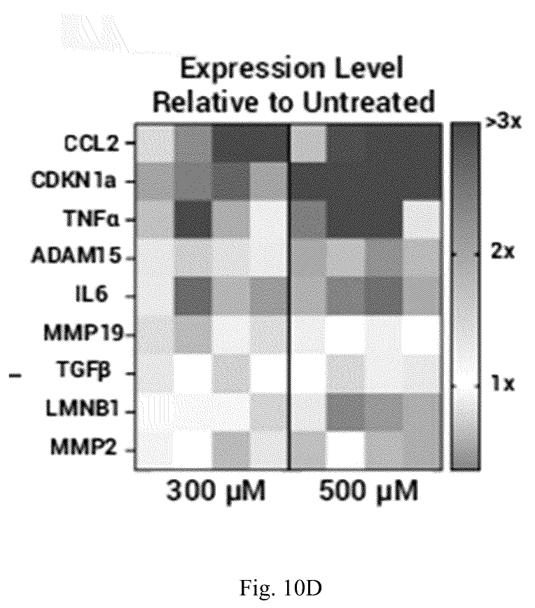

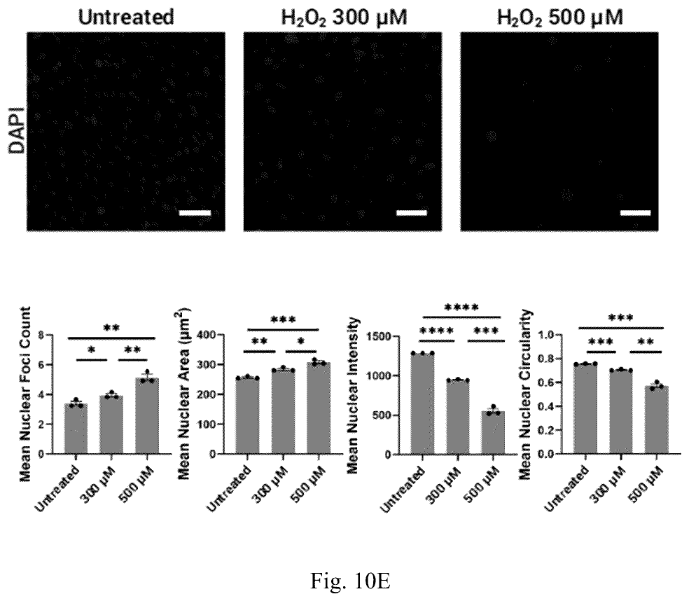

To study the nuclear phenotypic changes associated with senescence, an in vitro culture system was designed to induce senescence in a myoblast cell line (C2C12) using hydrogen peroxide (H₂O₂) to stimulate oxidative stress and damage (FIG. 10A) (Wang, Z., et al., Methods Mol. Biol., 2013). This treatment led to a decrease in cells expressing the proliferation marker Ki67, an increase in the expression of the DNA damage marker γH2AX, and an increase in SA-β-gal activity, all in a dose-dependent manner (FIG. 10B, FIG. 10C). The lack of Caspase-3 staining confirmed cells were not merely entering apoptosis (FIG. 10B). In addition, real-time quantitative PCR (qPCR) confirmed a SASP expression profile in treated cells (FIG. 10D). Consequently, an H₂O₂ treatment scheme was established that induced bona fide senescence and thus a robust system to study related nuclear morphological changes.

### Nuclear Morphometrics Identifies Bona Fide SnCs

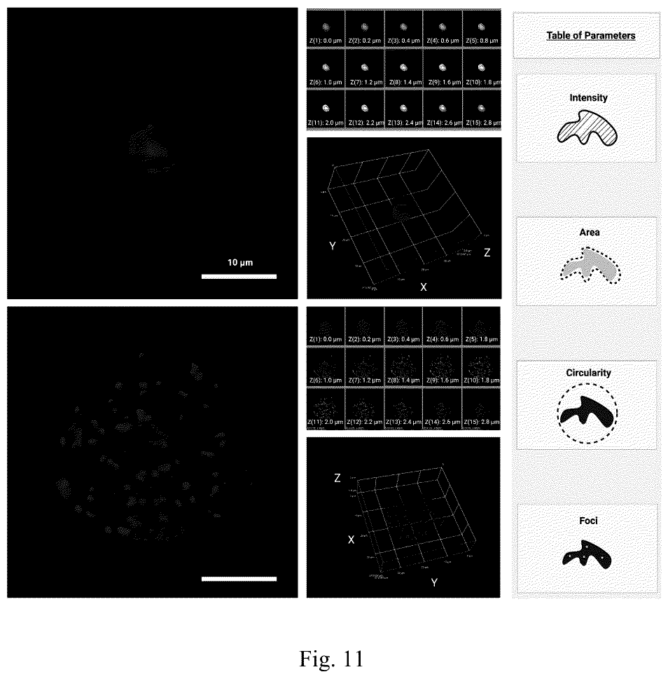

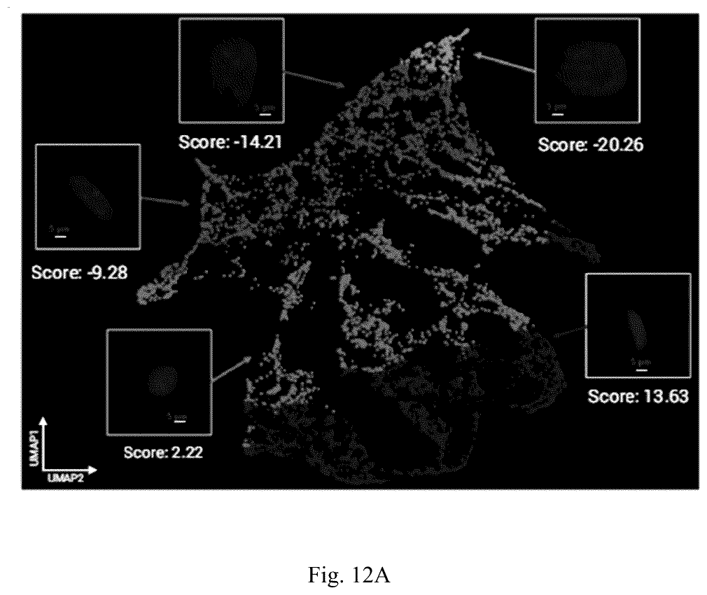

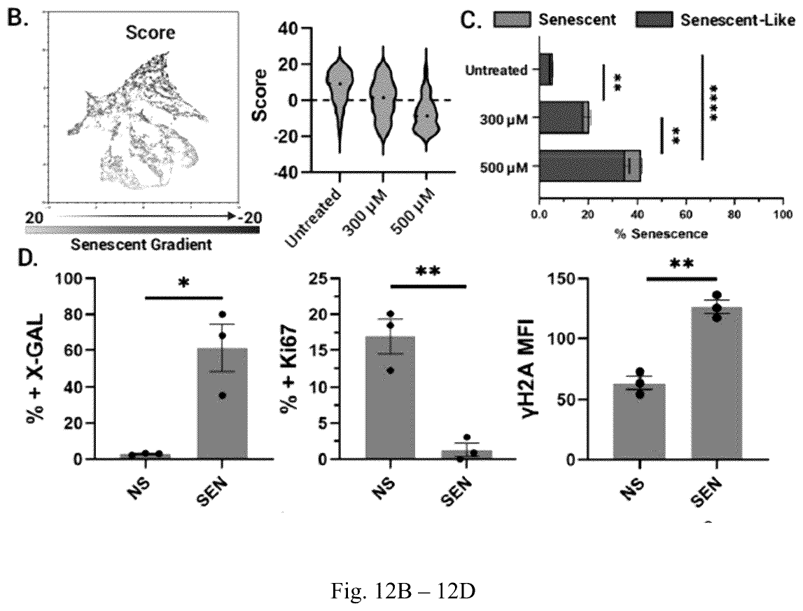

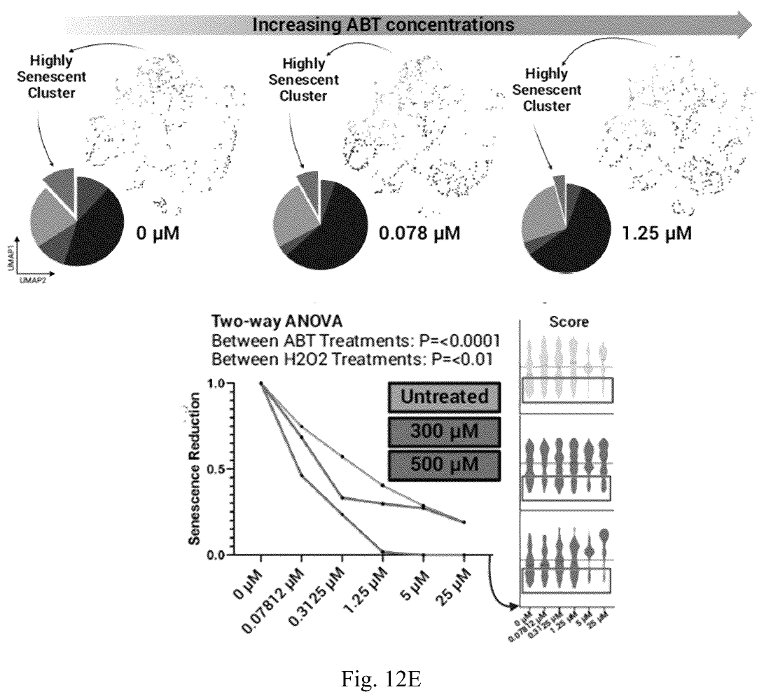

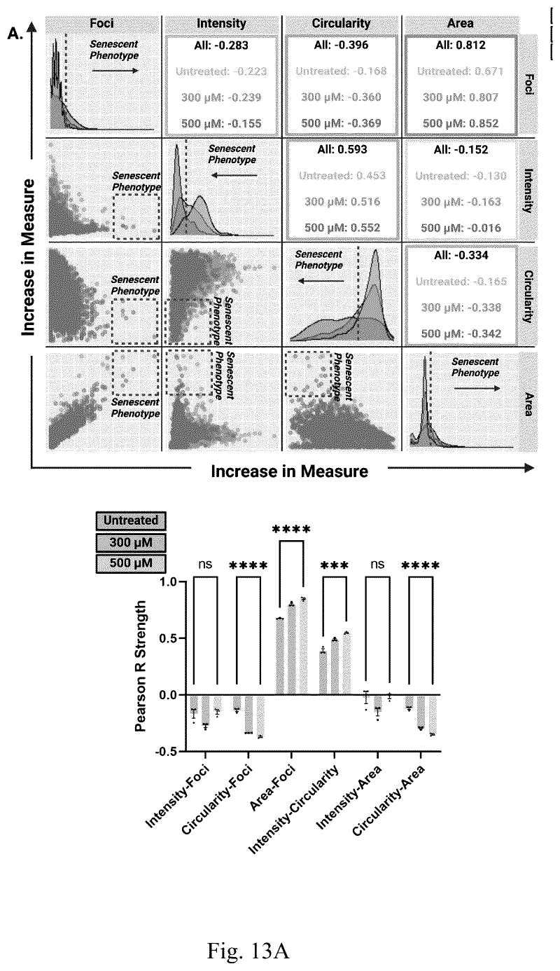

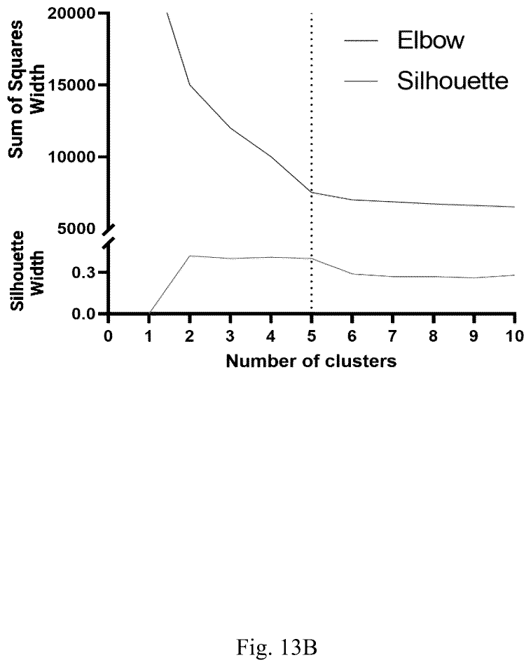

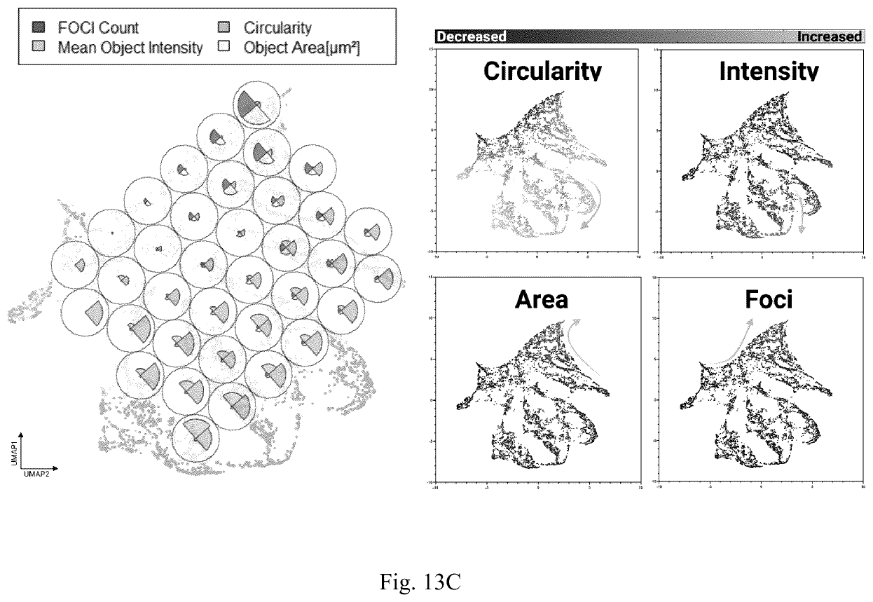

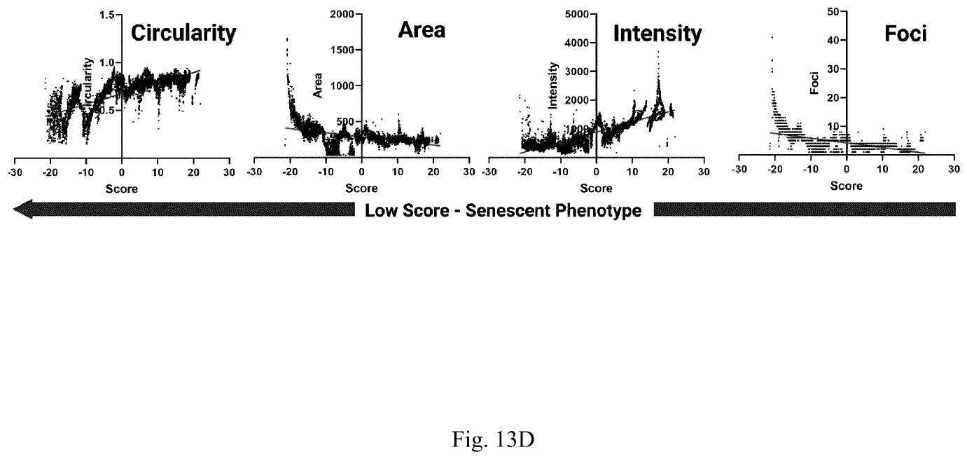

The nuclear phenotypes associated with increasing H₂O₂ treatment were then examined by staining with DAPI to provide an enhanced definition of nuclei. With increasing H₂O₂, an increase in nuclear size and dense foci were detected, as well as a concomitant decrease in nuclear circularity and DAPI intensity (FIG. 10E, FIG. 11), characteristics that have each independently been suggested to be associated with senescence (Moiseeva, V. et al. Nature, 2023; Freyter, B. M. et al., Cells, 2022; Zhang, R. et al., Dev. Cell, 2005). The correlation strength between paired parameters similarly increased with the greater concentration of H₂O₂, indicating that SnCs share many of these nuclear morphologies (FIG. 13A). To identify those cells that shared all parameters and were most indicative of senescence, a k-means unsupervised machine learning algorithm was applied to delineate groups with definitive morphometric similarity and determine the senescent nuclei that share all four parameters (FIG. 12A, FIG. 13C) (Ahmad, A., et al., Data Knowl. Eng., 2007). Clustering was performed on a normalized feature set consisting of all four parameters to preserve the variance within and between each morphometric feature. An elbow plot and silhouette method were both used to delineate the number of clusters (FIG. 13B). To generate a two-dimensional representation with single-cell resolution, these four parameters were dimensionally reduced using a Uniform Manifold Approximation Projection (UMAP) (FIG. 13C) (Stolarek, I., et al., iScience, 2022). Further reduction resulted in a one-dimensional “score” to achieve single-cell resolution for direct comparisons between nuclei within a UMAP, which established a “senescent gradient” (FIG. 12B and FIG. 13D). Using clusters identified by the k-means algorithm, a population with extreme morphometrics was identified and putatively labeled as bona fide SnCs. Another population that shared many of these traits, though exhibiting a less extreme phenotype, were deemed “senescent-like” cells (FIG. 12C). The percentage of cells identified as both senescent and senescent-like was greatest in the highest H₂O₂ treated samples (FIG. 12C). The cells bordering each cluster were most likely indicative of transitionary states between clusters, as they expectedly share partial phenotypic nuclear measures (FIG. 12A, FIG. 12B, FIG. 13C, FIG. 13D).

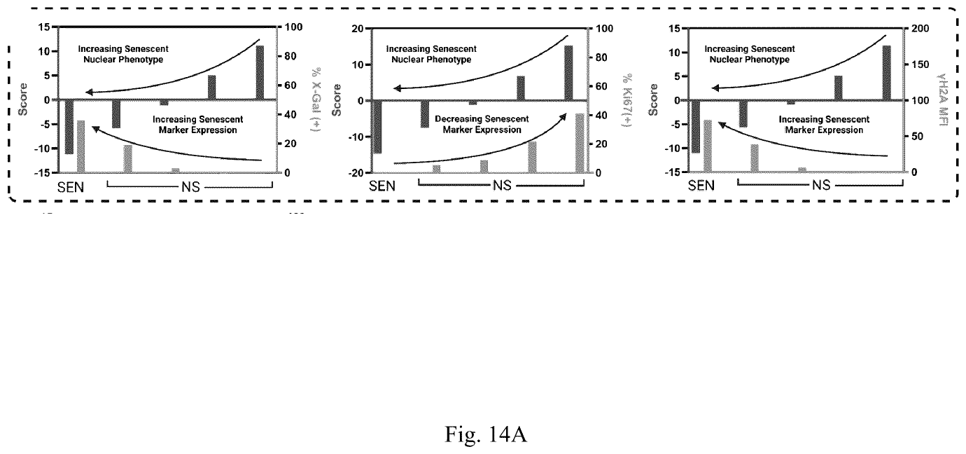

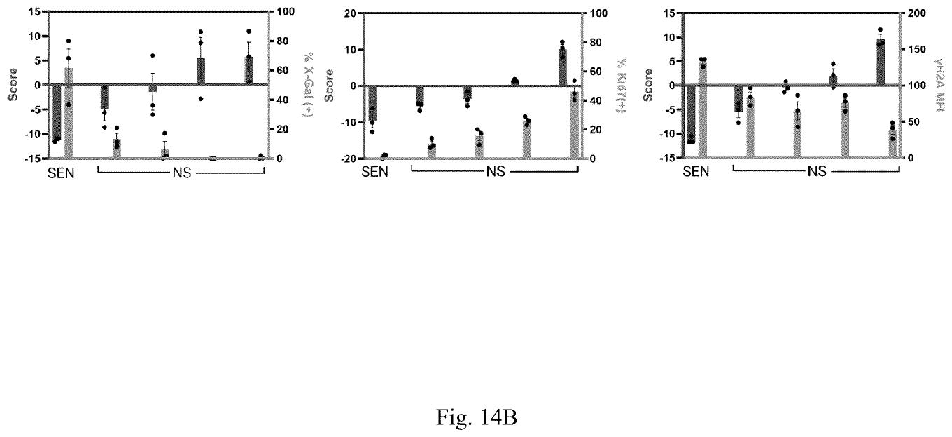

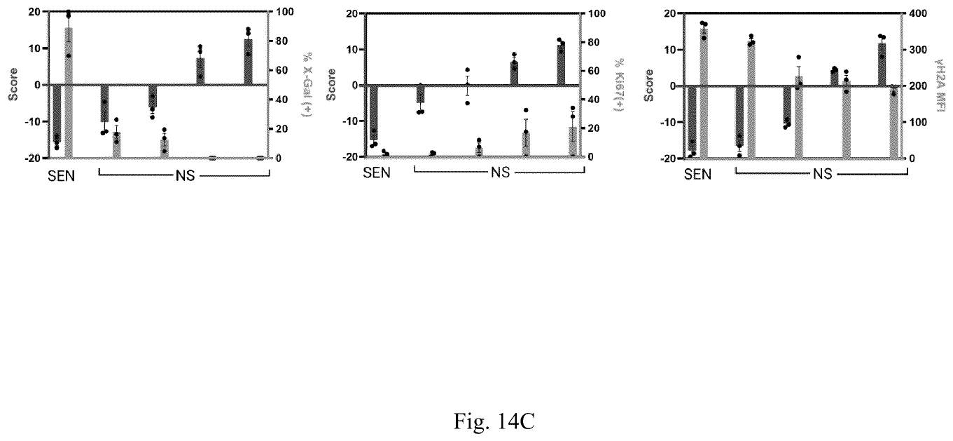

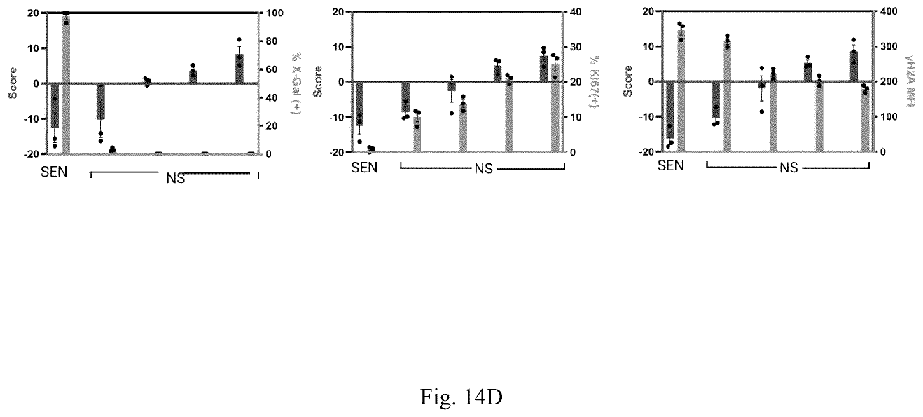

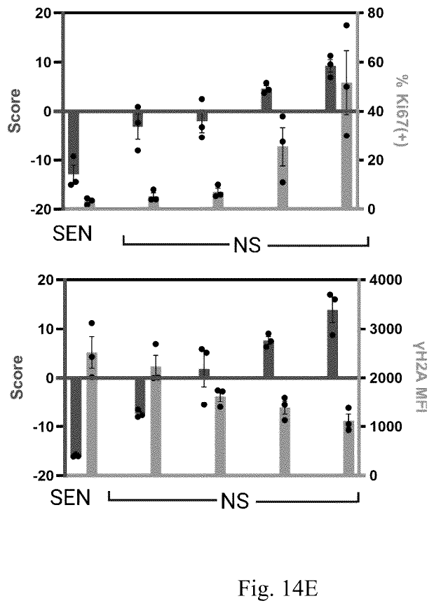

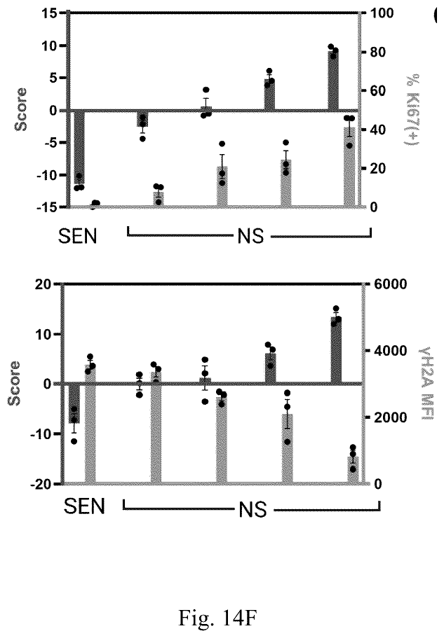

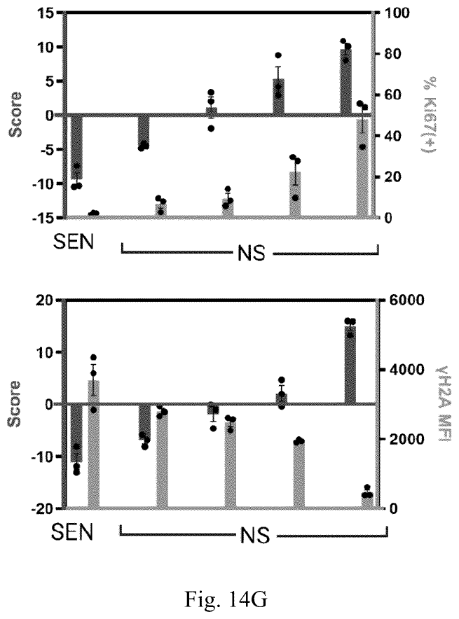

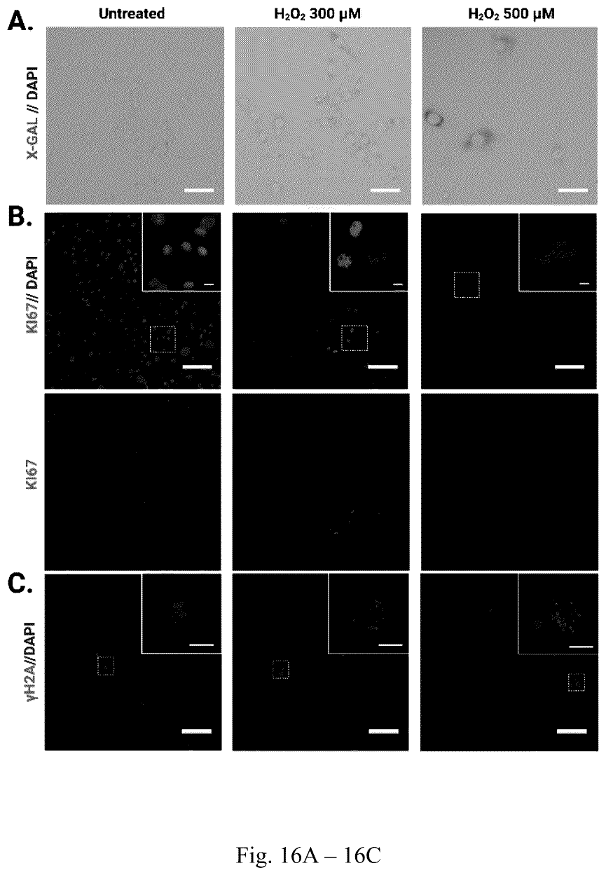

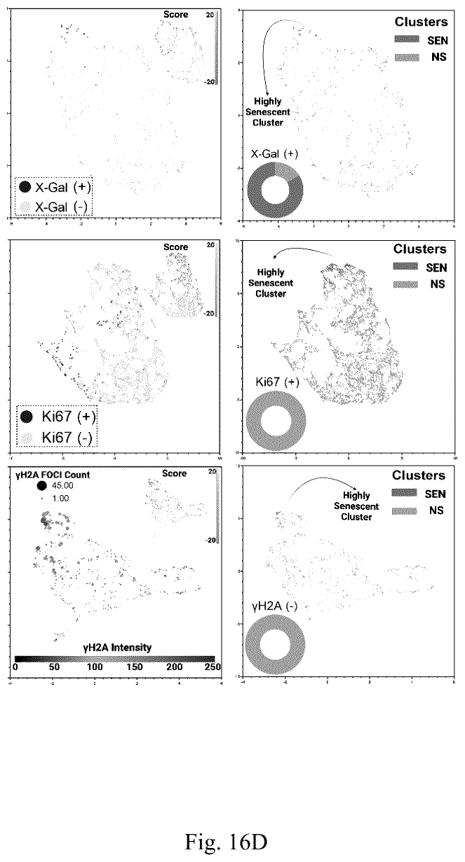

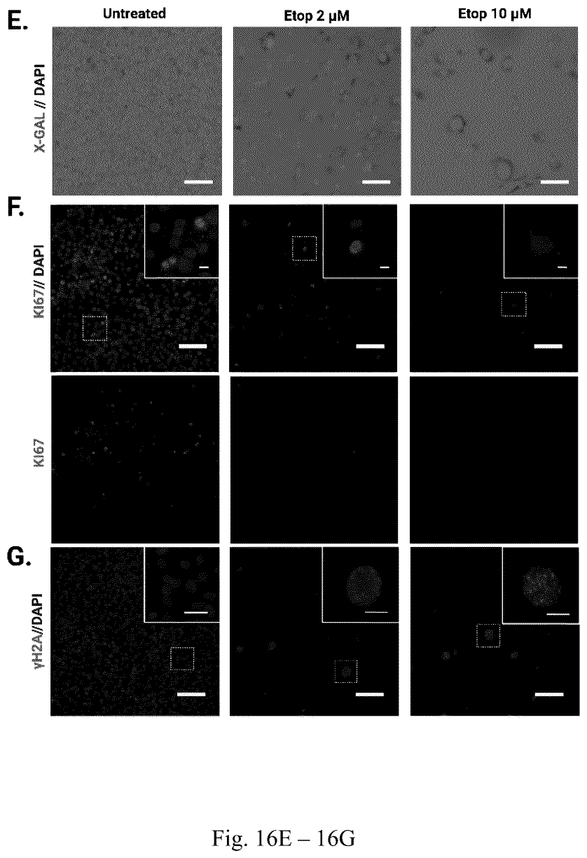

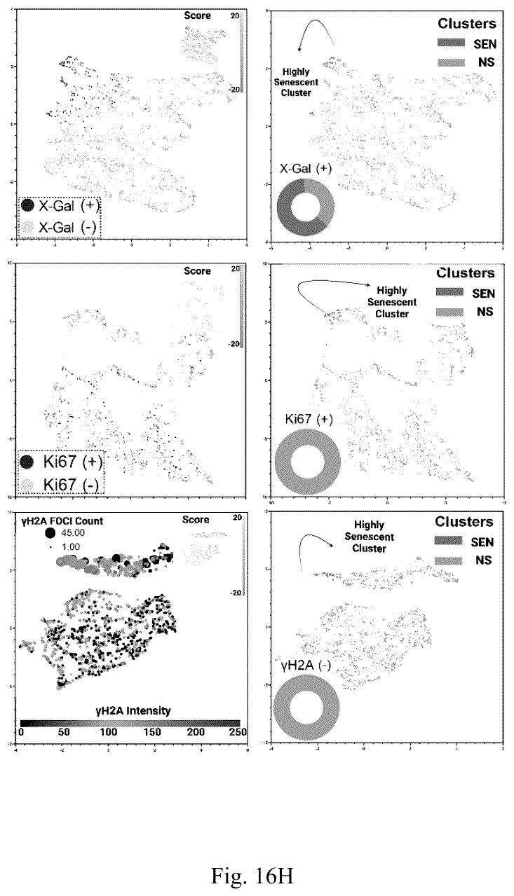

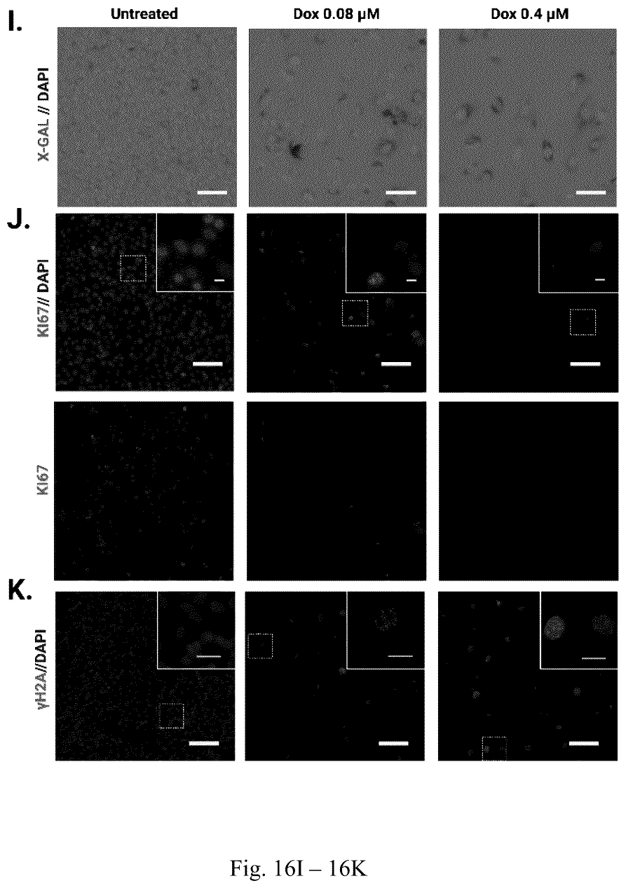

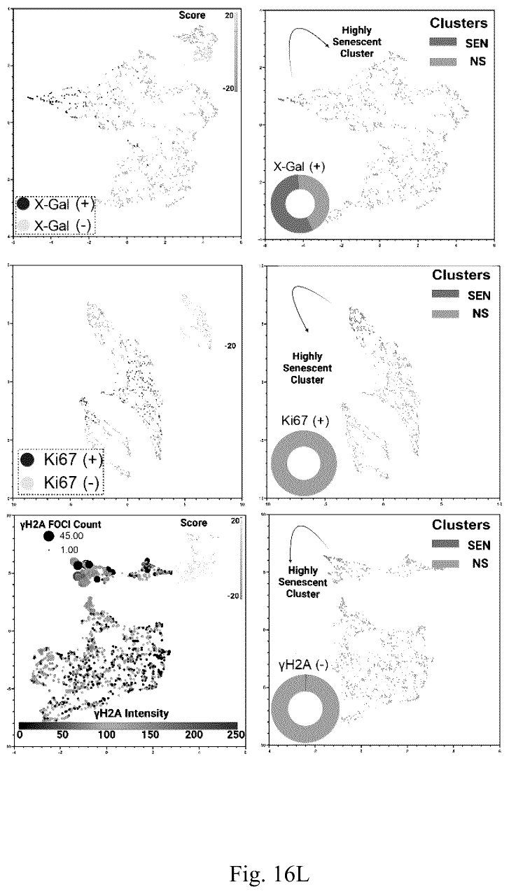

To confirm that cells clustered by the nuclear morphometric pipeline (NMP) were genuinely senescent, their cell cycle status (Ki67), degree of DNA damage (γH2AX), and SA-β-gal activity were assessed (FIG. 12D). The senescent nuclei identified by the NMP demonstrated a decrease in the Ki67 staining, an increase in γH2AX mean fluorescence intensity (MFI), and an increase in SA-β-gal activity (FIG. 12D, FIG. 16A, FIG. 16B, FIG. 16C, and FIG. 16D), collectively supporting the senescent phenotype. Specifically, less than 3% of nuclei in the non-senescent clusters were X-Gal positive, less than 2% of nuclei in the senescent cluster were Ki67 positive, and there was a two-fold increase in γH2AX expression from the non-senescent to senescent clusters (FIG. 12D). Each non-senescent cluster was also examined, demonstrating a gradient of gene expression consistent with a gradient of phenotypes leading to senescence (FIG. 14A, FIG. 14B). In addition, treatment with the senolytic Navitoclax (ABT-263), resulted in a dose-dependent reduction in the cells with nuclei in the senescent cluster, with the greatest lysis in the highest H₂O₂ treated samples (FIG. 12E).

The NMP Captures SnCs from Various Inducers and Cell Lines

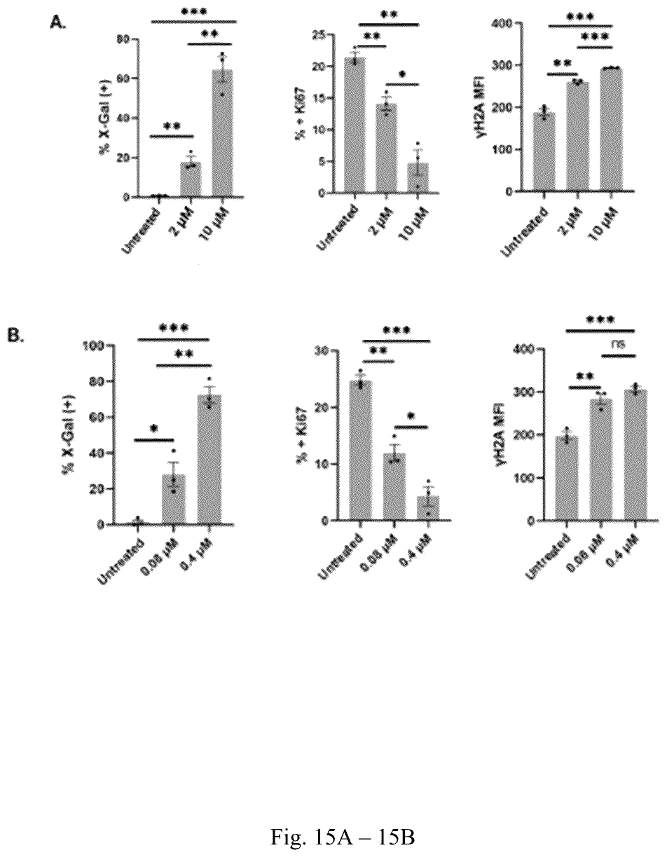

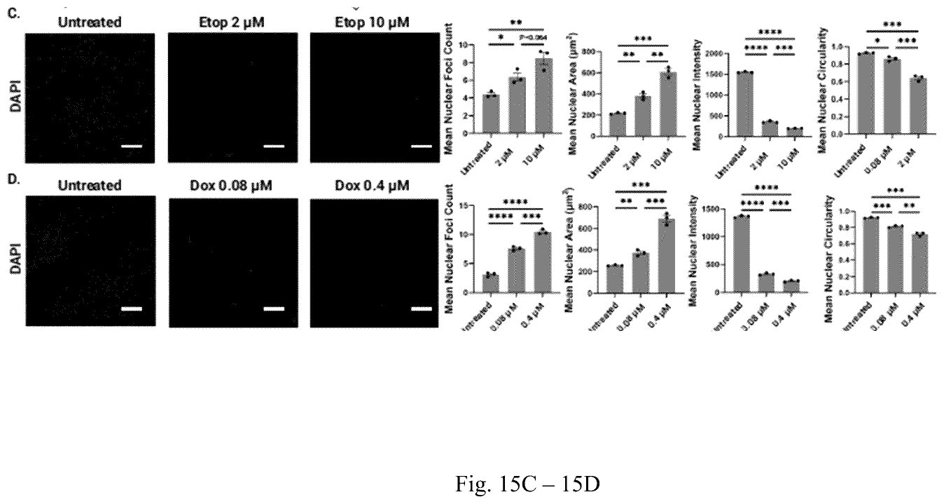

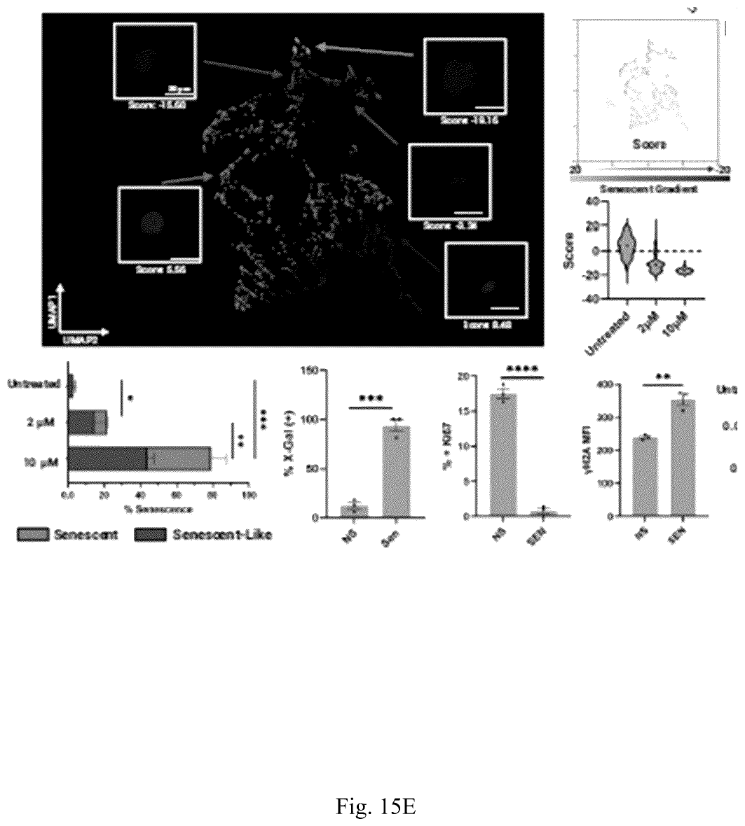

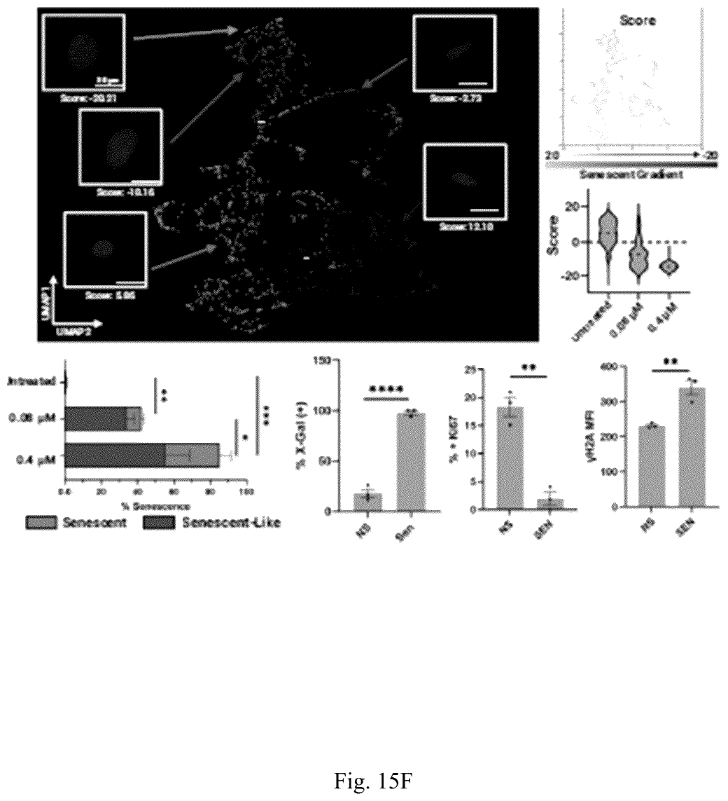

To examine the efficacy of the pipeline across mechanisms of senescence induction, the senescence inducers were expanded to include etoposide and doxorubicin as alternatives to H₂O₂ (FIG. 15A-FIG. 15F and FIG. 16A-FIG. 16K). Etoposide and doxorubicin both act as topoisomerase II inhibitors, causing the accumulation of DNA damage, which leads to the DNA damage response (DDR) (Montecucco, A., et al., EXCLI J., 2015). Doxorubicin has the added impact of increasing oxidative stress (Linders, A. N. et al., npj Aging, 2024). Thus, both are robust inducers of senescence at established concentrations. Following treatment of C2C12s with each of these small molecules, senescence induction was confirmed in a dose-responsive manner by verifying cell cycle exit (deceased Ki67+ cells), increased DNA damage (increased γH2AX staining), and increased SA-β-gal activity (FIG. 15A, FIG. 15B). Next, a shift in nuclear morphology was established with each treatment consistent with the senescent phenotype, demonstrating an increase in nuclear size and dense foci, and a decrease in nuclear circularity and DAPI intensity (FIG. 15C, FIG. 15D). The NMP was then deployed to assess nuclei treated with etoposide (FIG. 15E, FIG. 16E, FIG. 16F, FIG. 16G, and FIG. 16H) or doxorubicin (FIG. 15F and FIG. 16I, FIG. 16J, FIG. 16K, and FIG. 16L), generating a UMAP to visualize a gradient of dimensionally reduced nuclear morphometrics. The cluster denoted as senescent exhibited features of cell cycle exit (decreased Ki67), DNA damage (increased γH2AX MFI), and lysosomal accumulation (increased SA-β-gal activity), proving the NMP can effectively identify SnCs originating from differing mechanisms (FIG. 15E, FIG. 15F).

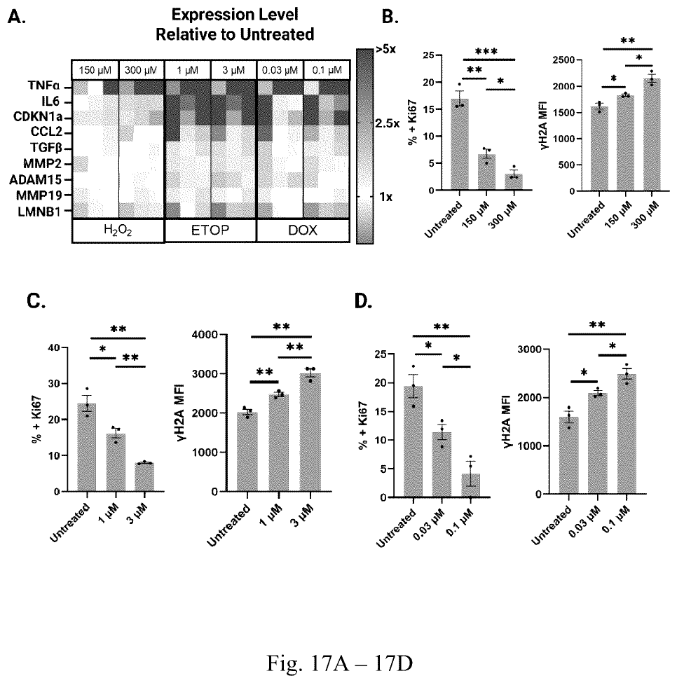

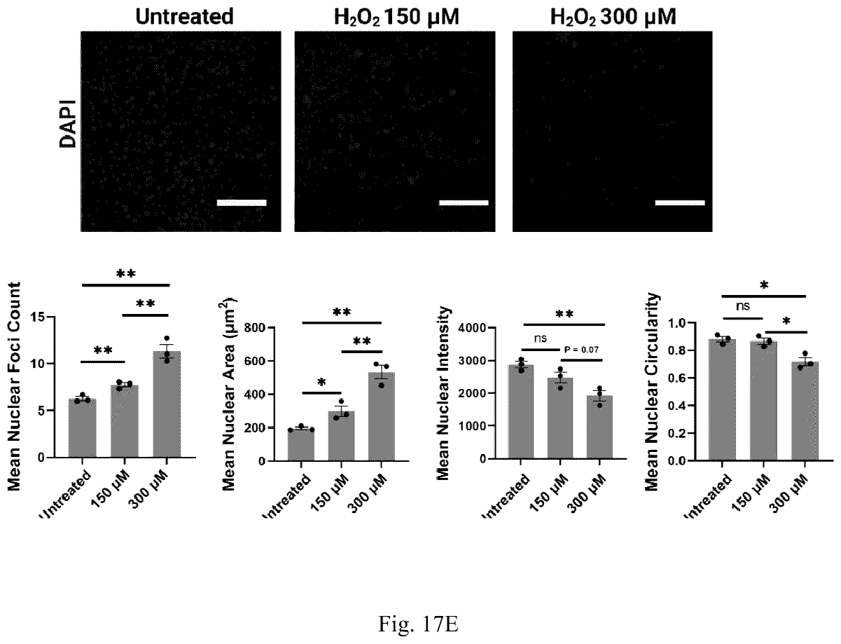

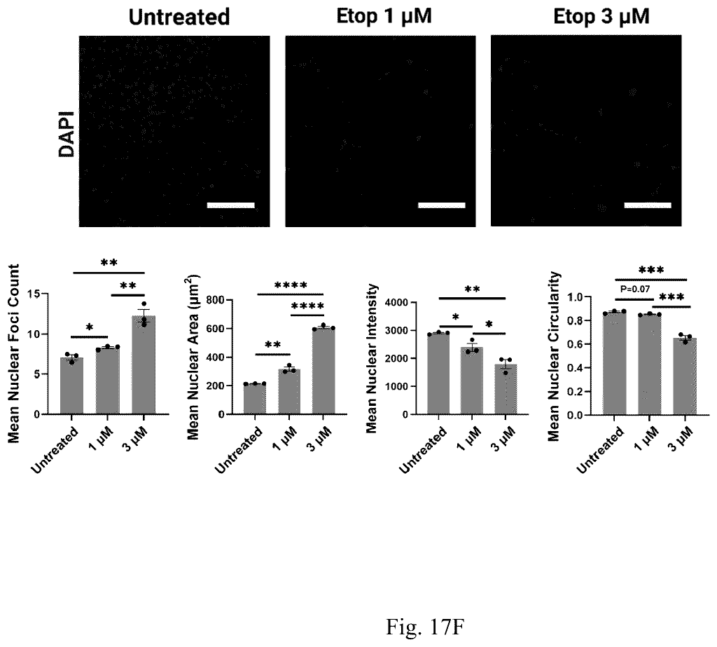

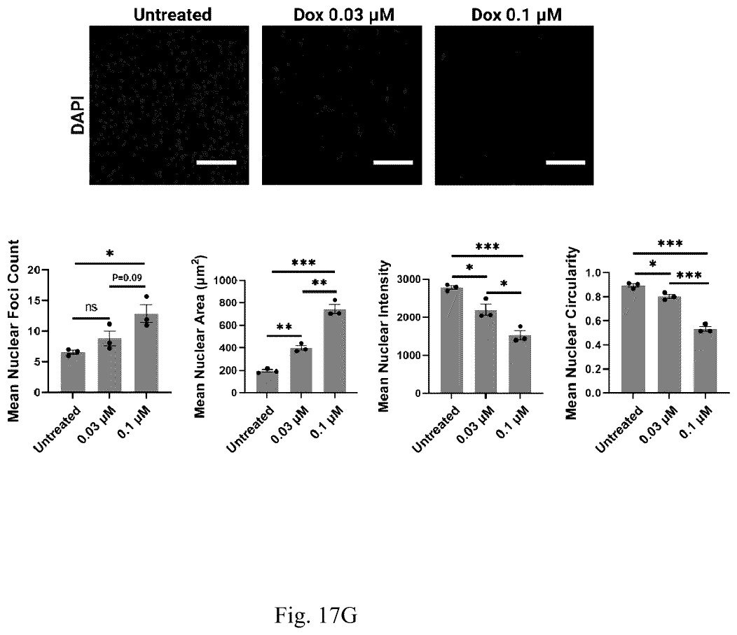

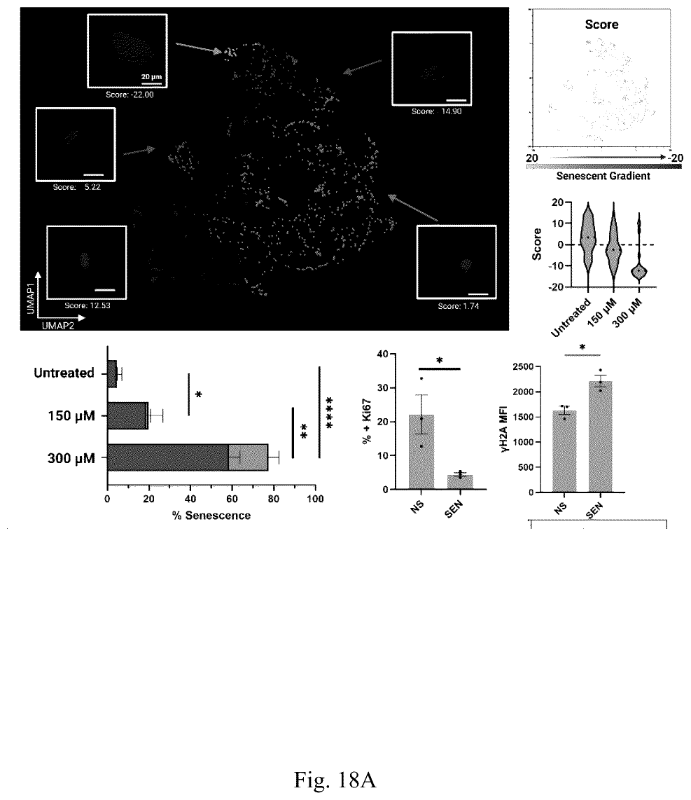

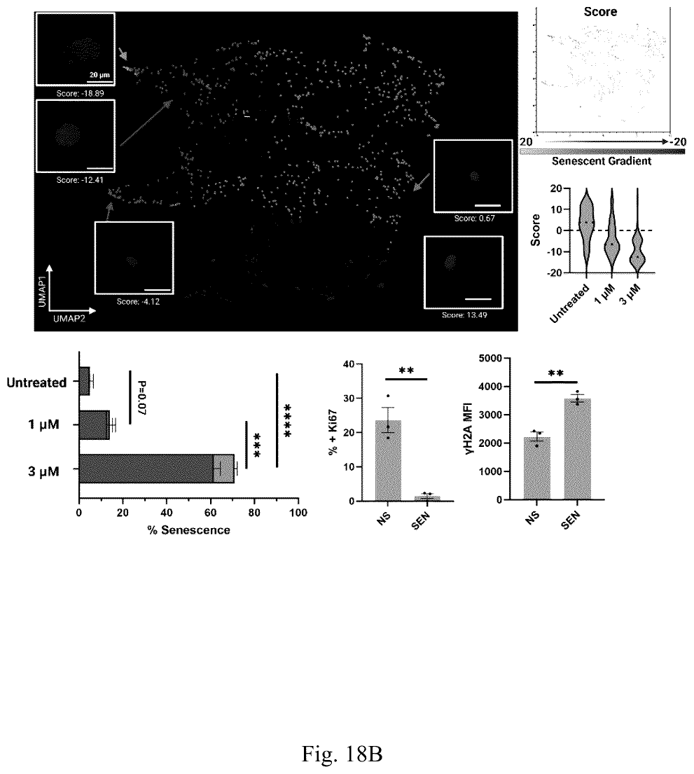

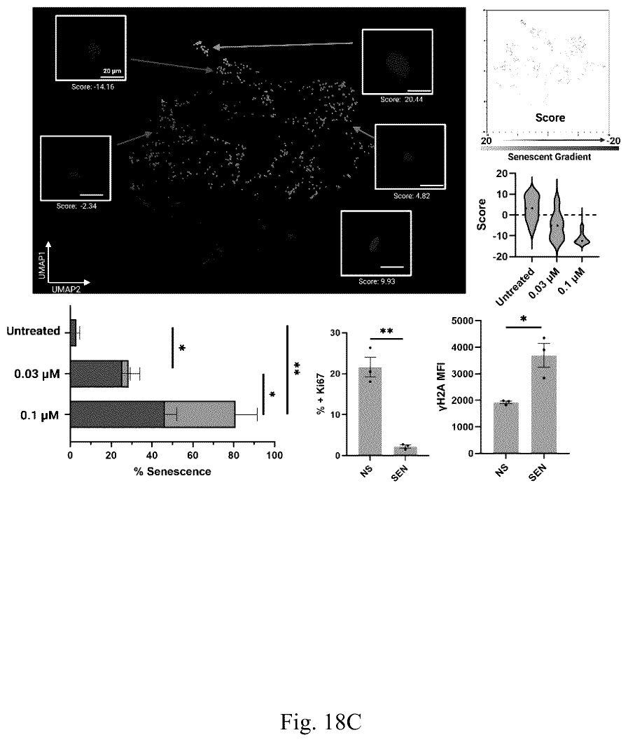

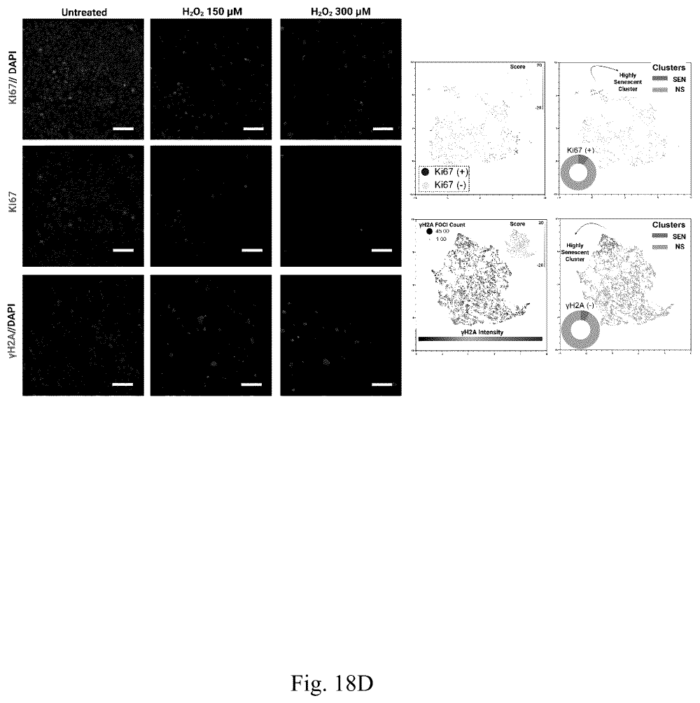

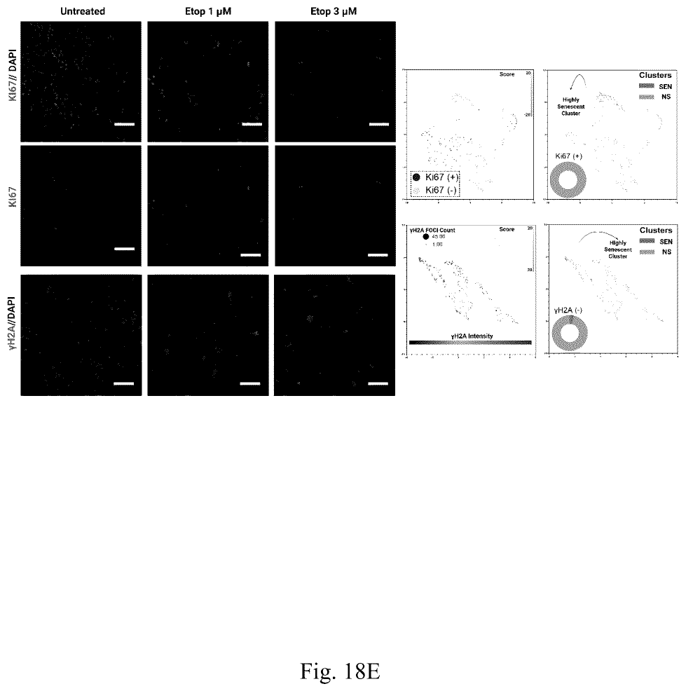

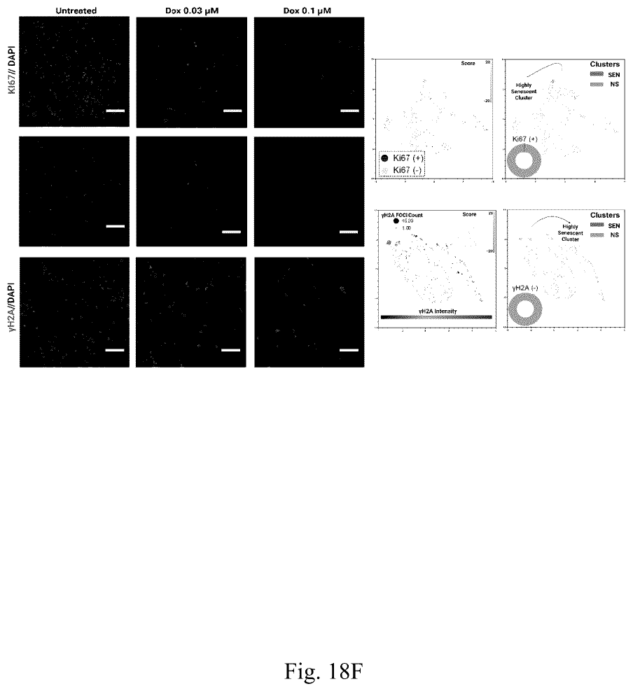

It was hypothesized that the four measured nuclear morphometrics are also conserved across cell types undergoing senescence, providing the NMP with broad applicability. To test this premise, the cell line, 3T3-L1 preadipocytes, was treated with H₂O₂, etoposide, and doxorubicin (FIG. 17). 3T3-L1s were chosen due to previous research suggesting a robust senescence response in adipose tissue, and thus, potential biological relevance (Nerstedt, A., et al., J. Cell Commun. Signal., 2023). The induction of senescence was confirmed in all treatments in a dose-dependent manner, with SASP profiling by real-time qPCR (FIG. 17A), a decrease in expression of Ki67, and an increase in γH2AX (FIG. 14E, FIG. 14G, FIG. 17B, FIG. 17C, and FIG. 17D). Consistent results were not achieved with SA-β-gal staining of senescent 3T3-L1s, emphasizing the importance of developing new methods of identifying SnCs across cell types. The nuclear morphological changes, as described before, aligned with increasing treatment of H₂O₂ (FIG. 17E), etoposide (FIG. 17F), and doxorubicin (FIG. 17G). Clustering by the NMP demonstrated a dose-dependent increase in senescent-like and senescent nuclei as well as a corresponding decrease in scores (FIG. 18A, FIG. 18B, and FIG. 18C). Clusters were confirmed to be senescent by the decrease in Ki67 expression and increase in γH2AX expression at single-cell resolution for H₂O₂ (FIG. 18D), etoposide (FIG. 18E), and doxorubicin (FIG. 18F) treatments.

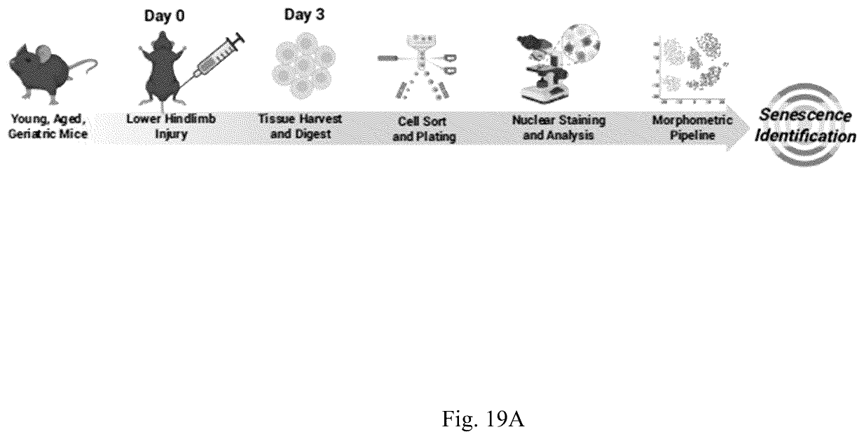

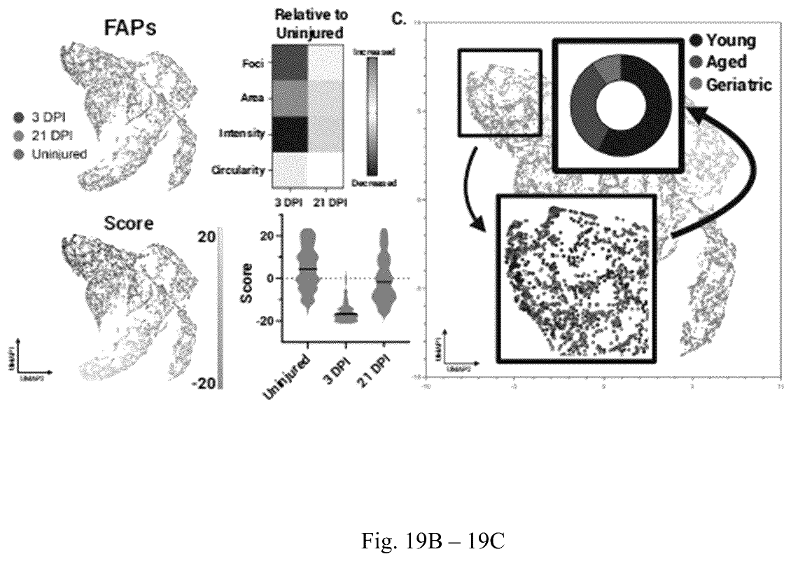

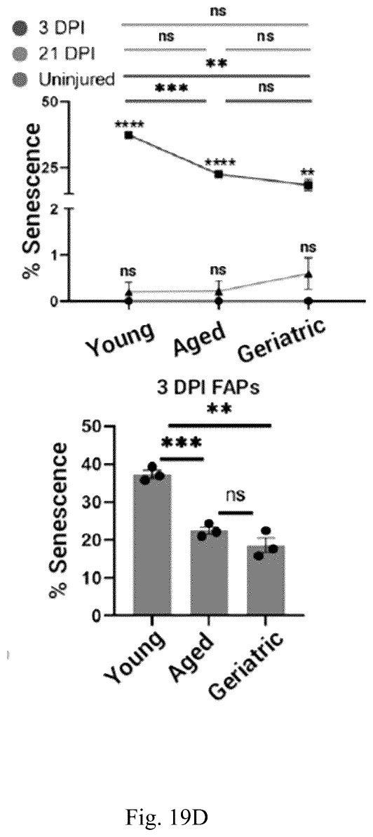

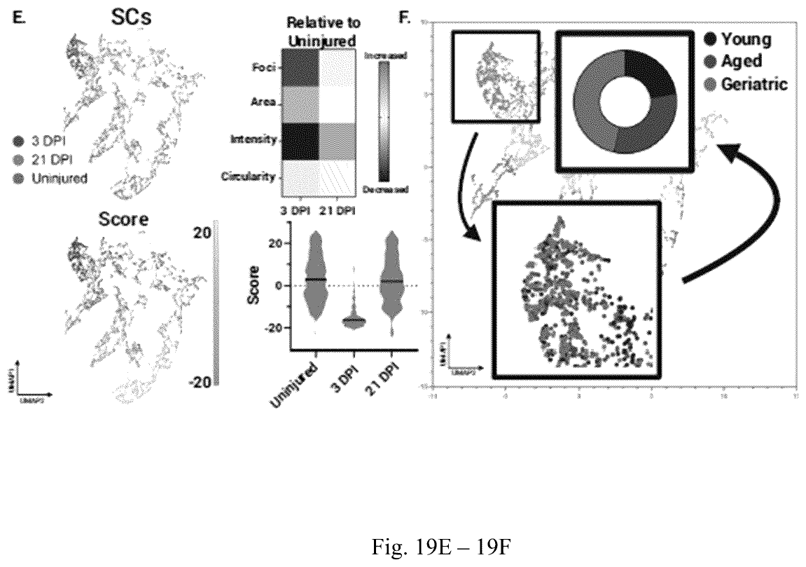

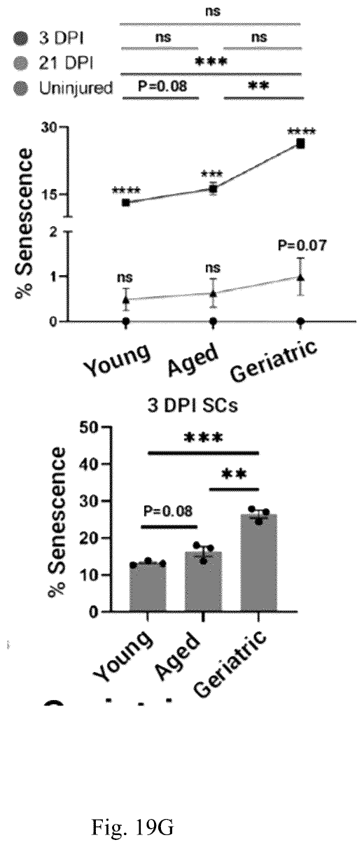

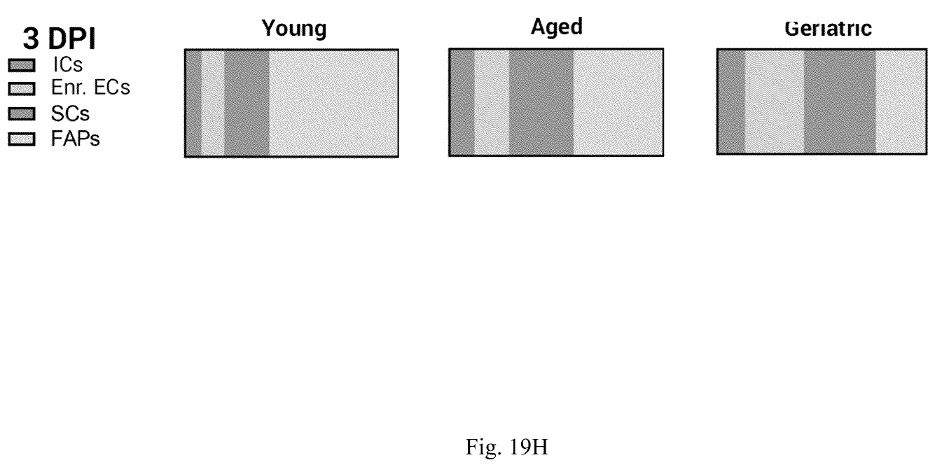

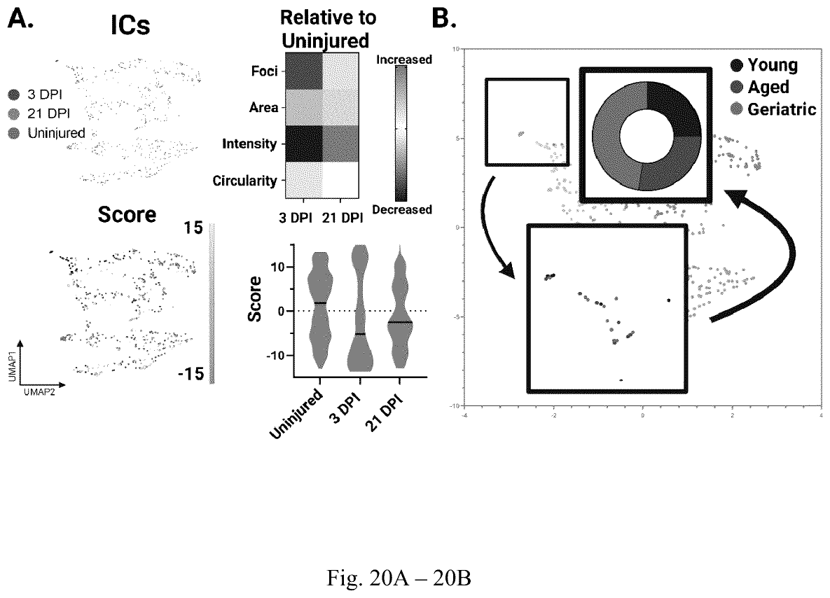

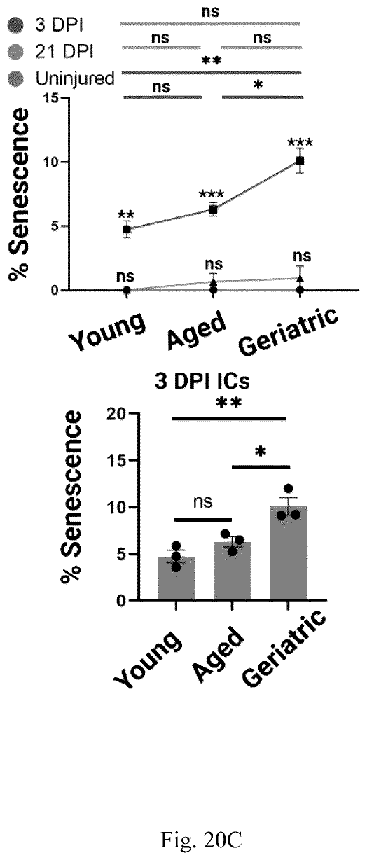

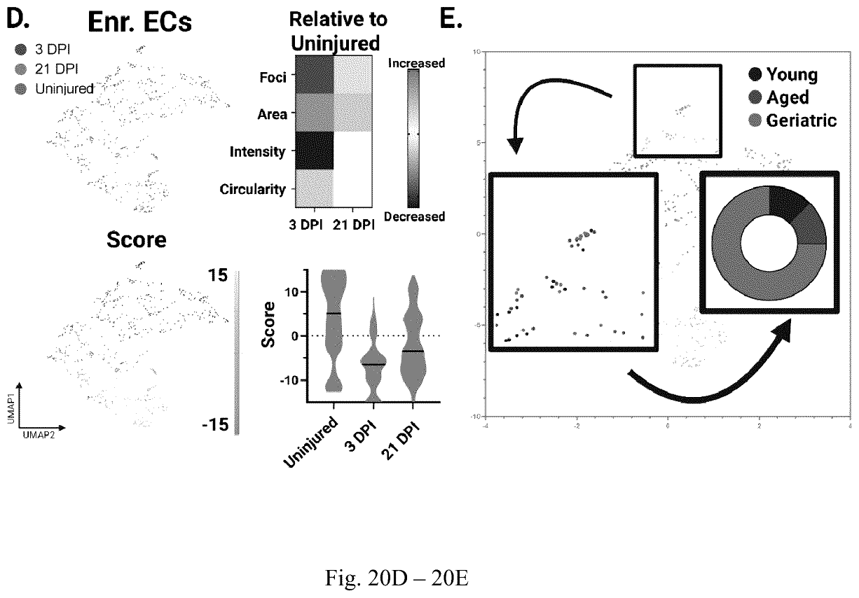

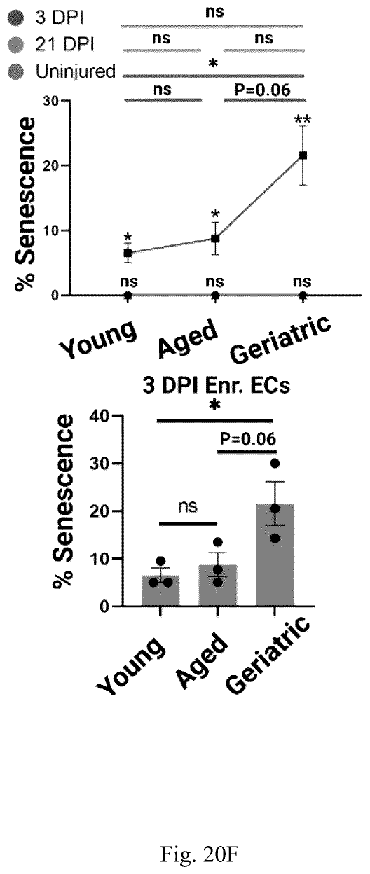

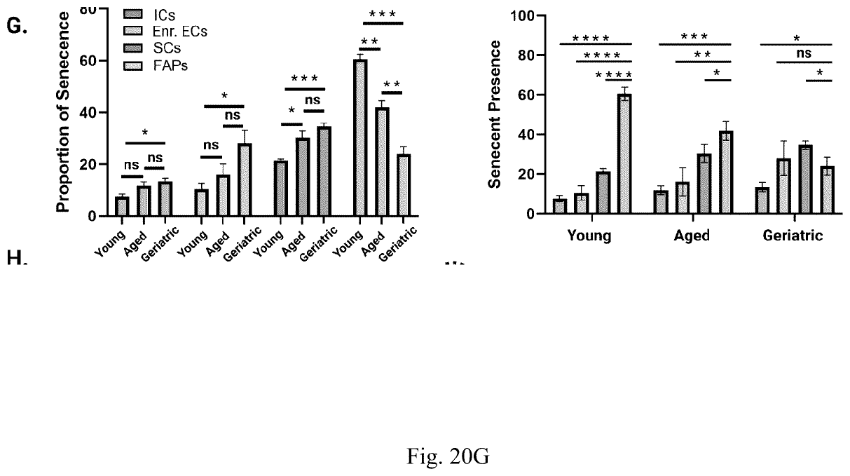

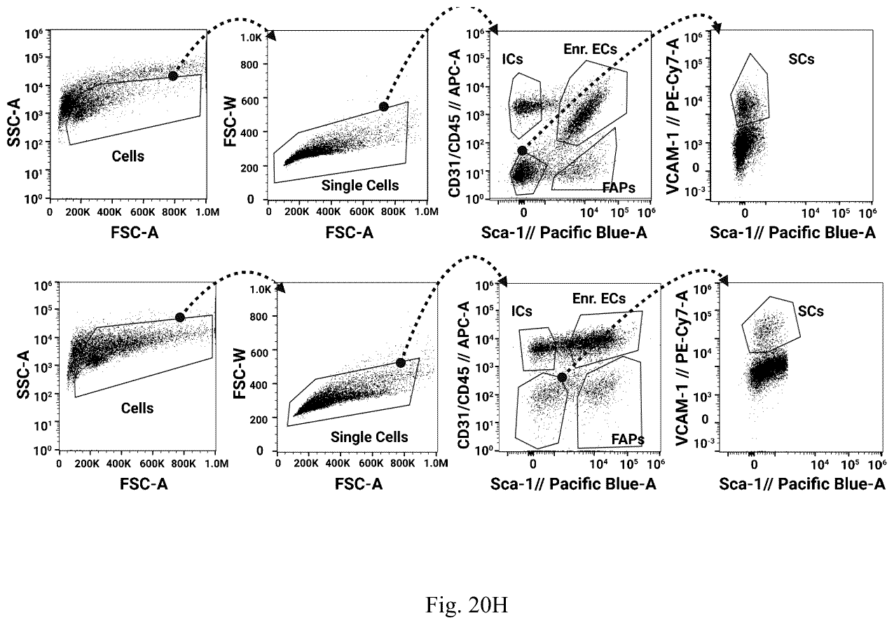

Age and injury shape SnC dynamics during muscle regeneration To translate the NMP to the in vivo application and identification of SnCs, homeostatic and regenerating SkM, a tissue reported to contain SnCs in both contexts across age, was examined (FIG. 19A-FIG. 19H, FIG. 20A-FIG. 20H) (Wan, M., et al., Bone Res., 2021). To induce regeneration, BaCl₂ was injected intramuscularly into the lower hind limb muscles in young (5 months), aged (24 months), and geriatric (30 months) mice (FIG. 19A). Using the disclosed NMP, muscle stem cells (aka satellite cells—SCs), mesenchymal stem cells (MSCs, aka fibroadipogenic progenitors-FAPs), an enriched fraction of endothelial cells (Enr-ECs), and immune cells (ICs) were analyzed that were isolated by FACS from uninjured and injured tissue at 3 days post-injury (DPI), a time when the mononuclear cellular response is at its greatest (Wosczyna, M. N. et al., Cell Rep., 2019), and at 21 DPI when muscle regeneration is nearly complete except for continued remodeling and hypertrophy (FIG. 19A-FIG. 19H and FIG. 20A-FIG. 20H) (Baghdadi, M. B. et al., Dev. Biol., 2018).

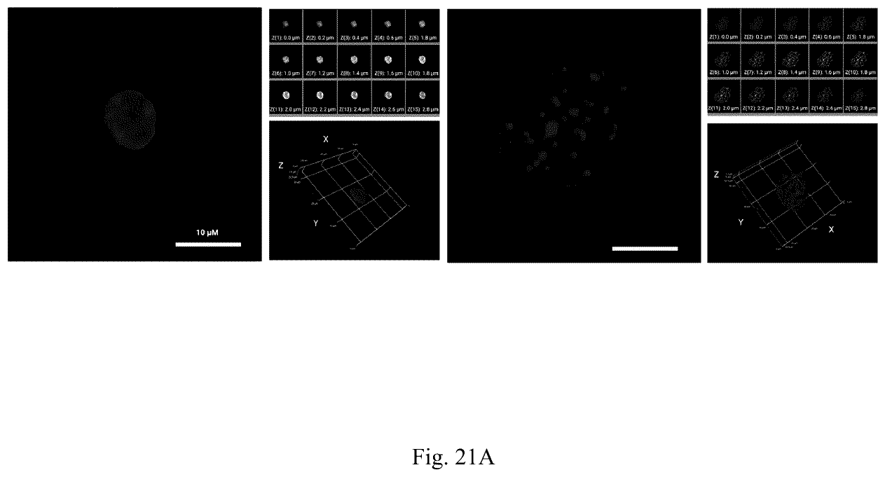

Following the dimensional reduction of the four nuclear morphometrics for each cell population from uninjured, 3 DPI, and 21 DPI, a spectrum of non-SnCs to SnCs was clearly identified, revealing a dynamic milieu of SnCs, both across the injury time course and across the age of mice (FIG. 19A-FIG. 19H and FIG. 20A-FIG. 20H). In homeostatic SkM, SnCs are essentially absent regardless of age, consistent with many recent studies and the evolving paradigm around senescence in uninjured tissue (FIG. 19D, FIG. 19G, FIG. 20C, FIG. 20F) (Moiseeva, V. et al. Nature, 2023). Following injury, there is a robust increase in SnCs at 3 DPI, supporting SnCs as necessary for efficient regeneration (FIG. 19D, FIG. 19G, and FIG. 20C, FIG. 20F, and FIG. 21A-FIG. 21B for detailed nuclear morphology). In young SkM, FAPs were the major fraction of SnCs, with SCs, Enr-ECs, and ICs only contributing a minority of the SnC population at 3 DPI (FIG. 16H and FIG. 20G). These data are consistent with a potential role for FAP senescence in young SkM regeneration. Intriguingly, while the senescence response in aged tissue remains robust at 3 DPI, SCs become the majority of SnCs, while FAPs fall into the minority in geriatric SkM (FIG. 19H and FIG. 20G). As SCs become more senescent in geriatric tissue, this is predicted to be detrimental to regeneration as it may limit the pool of myogenic precursors needed to recapitulate SkM (Liu, L. et al., Cell Stem Cell, 2018). While loss-of-function tests that target specific SnC populations in a temporal manner are needed to unambiguously determine their impact on SkM function, these data are consistent with senescence being evolutionarily maintained in animals and not selected against, inferring a beneficial role in tissue regeneration.

The disclosed data reveals a complex age-dependent SnC environment in early regeneration and denotes their seemingly beneficial and detrimental role in young and aged SkM, respectively (FIG. 19C, FIG. 19F, FIG. 20B, FIG. 20E). Interestingly, at 21 DPI, when muscle is largely rebuilt with regenerated myofibers and mononuclear cell numbers at homeostatic levels, SnCs have largely resolved, as demonstrated by the rebound seen in the scoring system (FIG. 19B, FIG. 19E, FIG. 20A, FIG. 20D) (Wosczyna, M. N., et al., Dev. cell, 2018). However, a small number of SCs remain senescent in geriatric SkM, reinforcing the potential impact of senescence in the myogenic precursor population, leading to less efficient regeneration in aged SkM and chronic inflammation from continued SASP expression (FIG. 19G). Thus, the disclosed data supports the necessity of FAP senescence in young tissue for efficient regeneration, a process that is blunted in aged tissue and concomitantly demonstrates that SCs acquire a senescent phenotype during aging, which is consistent with the reported age-dependent decrease in myogenicity of these cells (Zeng, W. et al., Dev. Cell, 2023).

### The NMP Identifies SnCs In Vivo Across Age

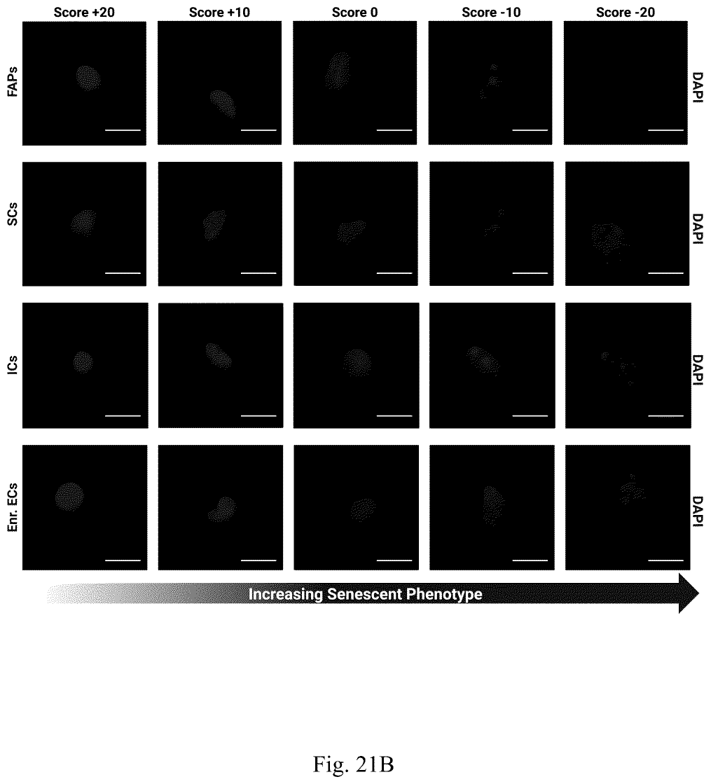

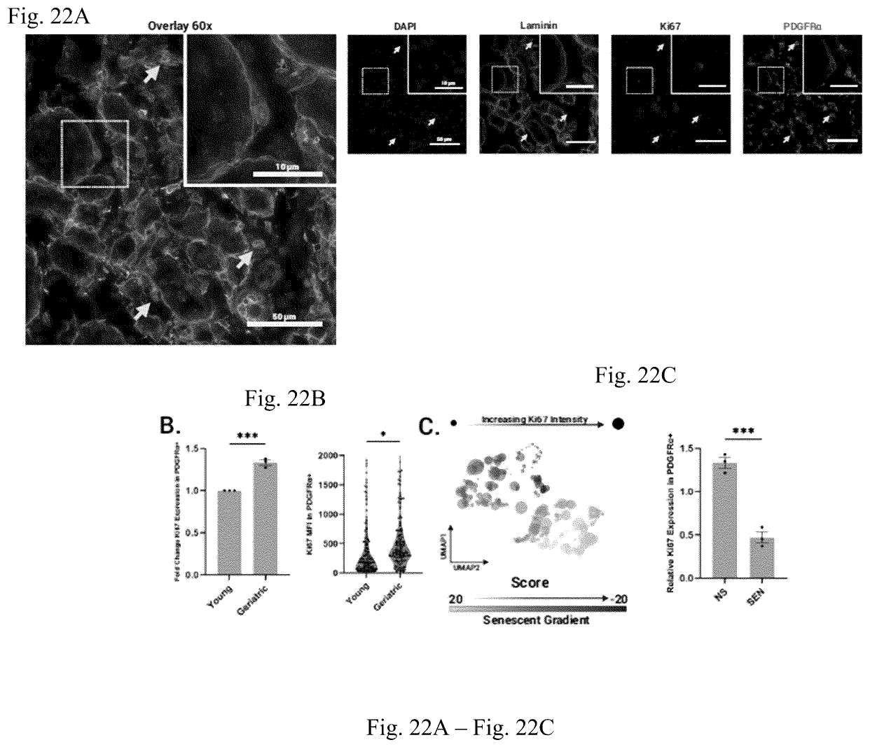

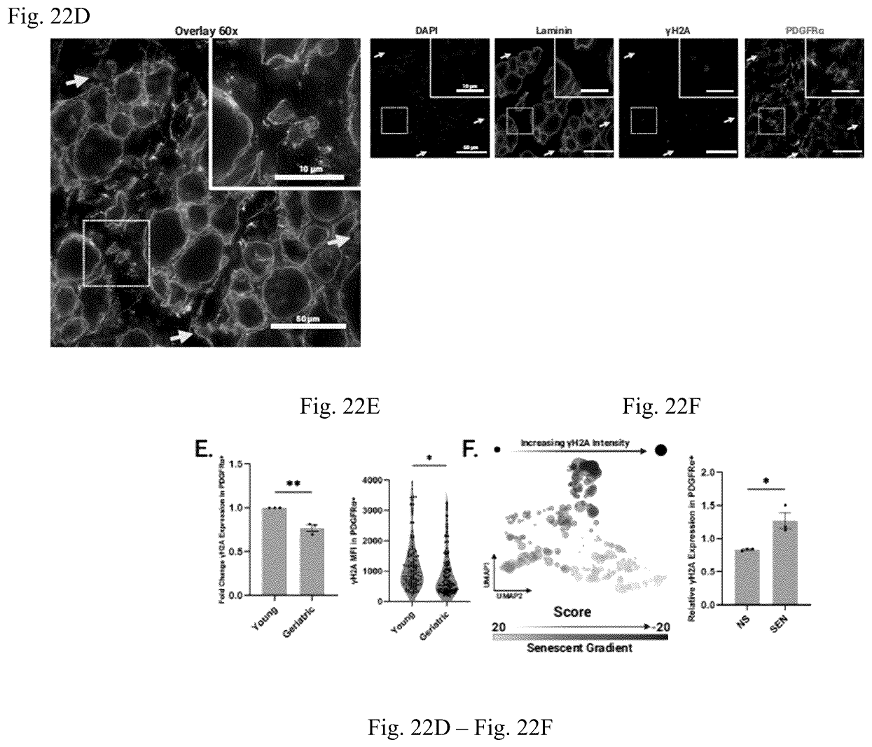

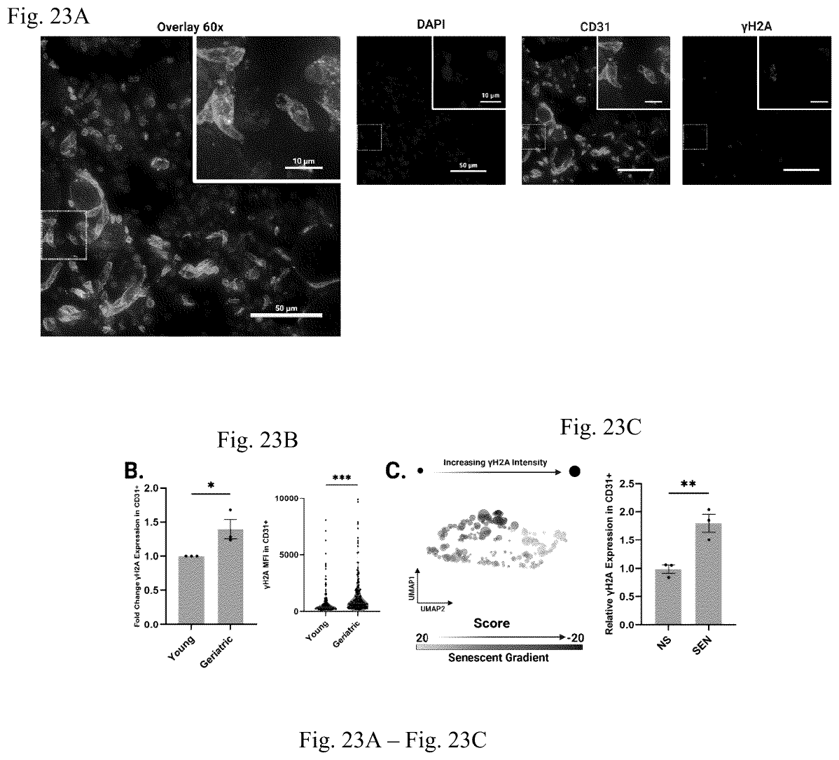

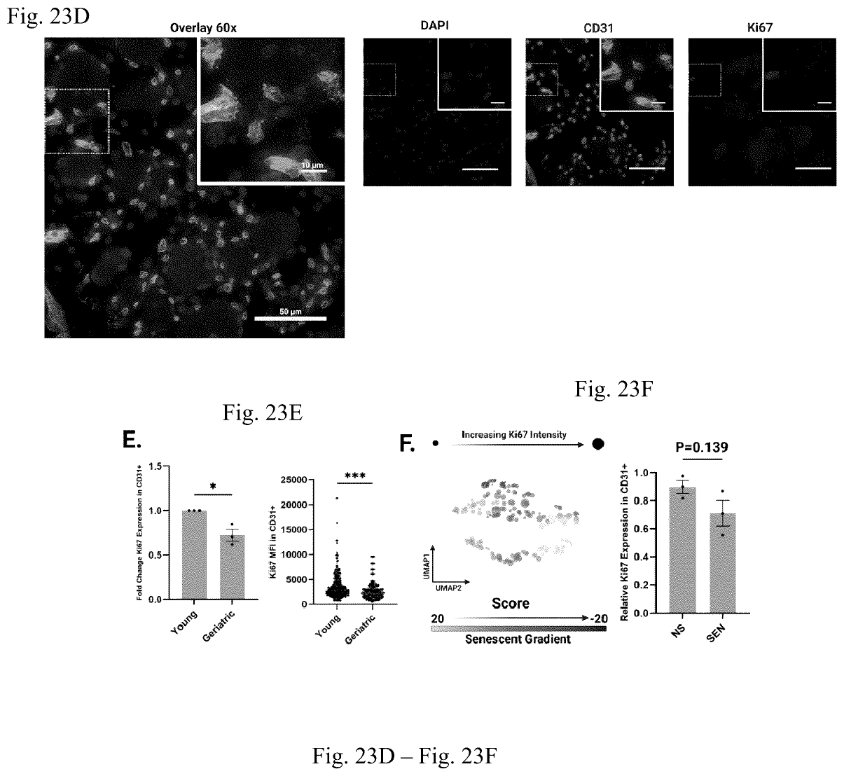

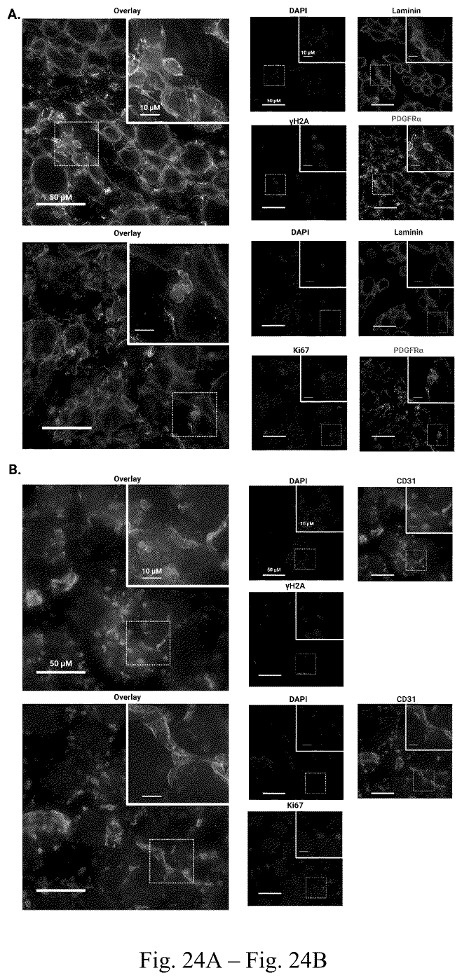

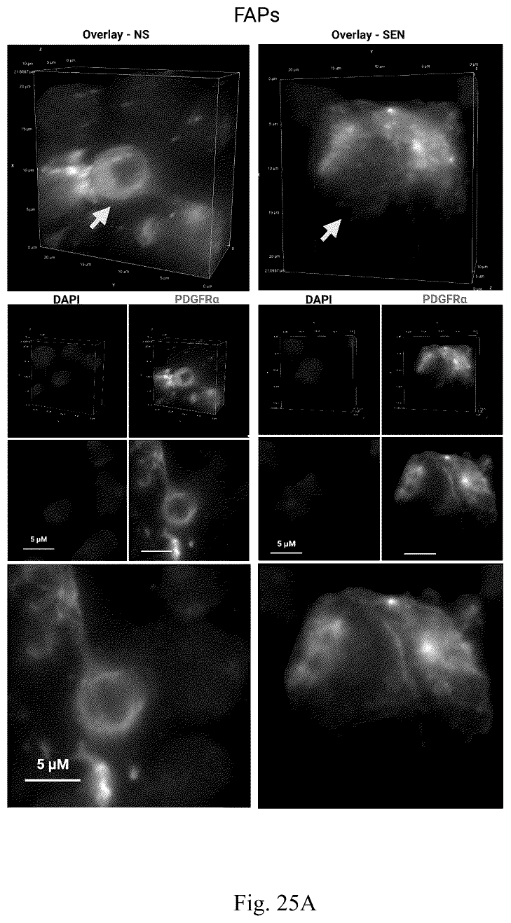

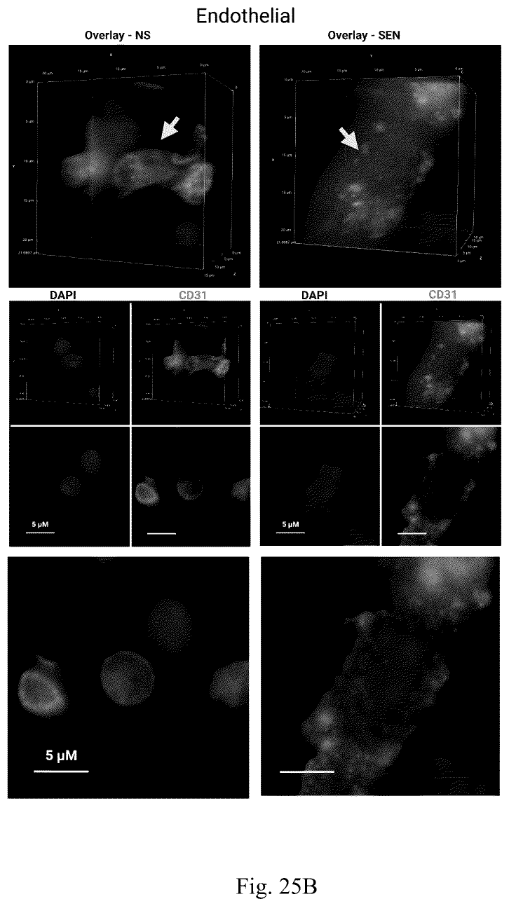

To verify the age-associated changes in the SnC population of SkM and to assess if the NMP can function with cells in vivo, cross-sections of injured SkM from young and geriatric mice were examined (FIG. 22A-FIG. 22F and FIG. 23A-FIG. 23F, FIG. 24, and FIG. 25 for detailed nuclear morphology). Injured SkM at 3 DPI was chosen due to the robust senescence response at this time and the dynamic change in the cell types that constitute the SnC population with age. Furthermore, FAPs were the focus of the study due to these being the majority component of the SnC population in SkM from young mice, while their representation substantially decreases in geriatric mice (FIG. 19C, FIG. 19H, and FIG. 20G). Using PDGFRα to identify FAPs in section, an increase in Ki67 expression (FIG. 22B) and decrease γH2A (FIG. 22E) was noted in FAPs of SkM from geriatric mice when compared to those of young (FIG. 24A and FIG. 25A for detailed nuclear morphology). Next the NMP was applied to the nuclei of PDGFRα+ FAPs in section measuring the four morphometrics and then these analyses were correlated with Ki67 and γH2A staining (FIG. 22C, FIG. 22F). The NMP correctly clustered senescent nuclei with these associated senescent markers confirming the dynamic decrease in FAPs with senescent characteristics with age and proving the applicability of the disclosed NMP to reveal genuine SnCs in vivo (FIG. 22C, FIG. 22F). An attempt was made to assess SCs in these tissues, but the 3 DPI environment proved inhibitory to Pax7 staining for the identification of SCs, even with the most current mouse-on-mouse and antigen retrieval techniques. It is postulated that the inflammatory milieu and low Pax7 expression in activated SCs prevent adequate identification at this stage. However, as the presence of senescent Enr-ECs also dramatically increased with age when comparing cells isolated from regenerating SkM of mice (FIG. 19H, FIG. 20D, FIG. 20E, FIG. 20F, FIG. 20G), it was decided to assess ECs in vivo as an alternative to SCs (FIG. 23, FIG. 24B, FIG. 25B for detailed nuclear morphology). Using CD31 to identify ECs in sections, an increase in γH2A (FIG. 23B) and decrease in Ki67 expression (FIG. 23E) was observed in ECs of regenerating geriatric SkM when compared to that of young. The NMP was deployed to assess EC nuclei, and those classified as SnCs contained significantly more γH2A (FIG. 23C) and tended to have less Ki67 (FIG. 23F) staining when compared to non-SnCs. These data further confirmed the applicability of the NMP to detect cells with senescent characteristics in vivo conditions while also being consistent with and thus supporting the ex vivo data demonstrating ECs senesce more in aged regenerative scenarios compared to young environments (FIG. 19H, FIG. 20D, FIG. 20E, FIG. 20F, and FIG. 20G).

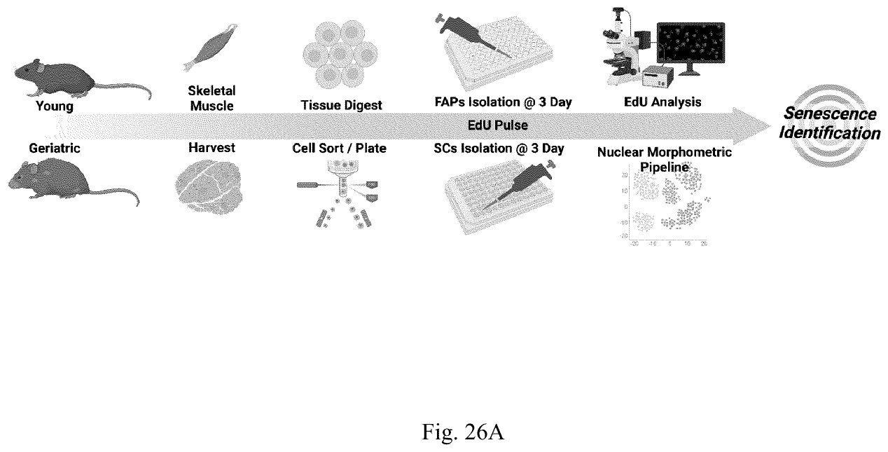

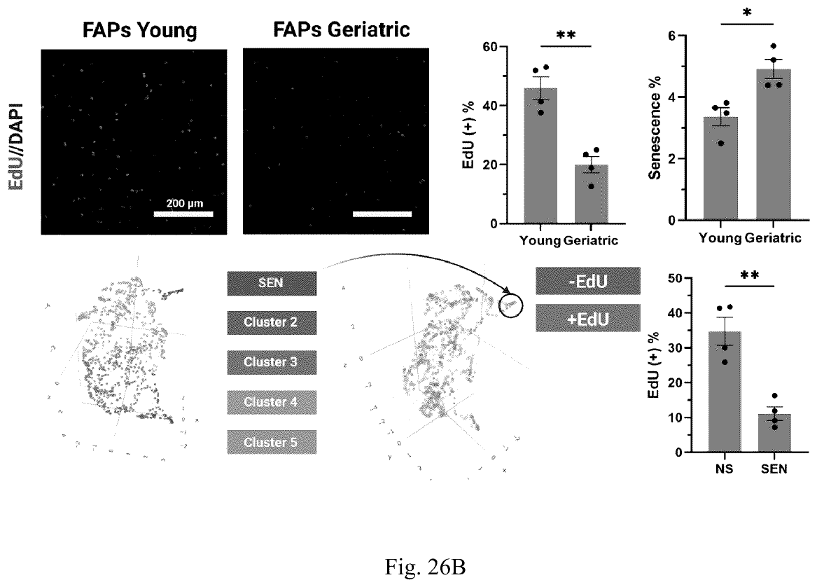

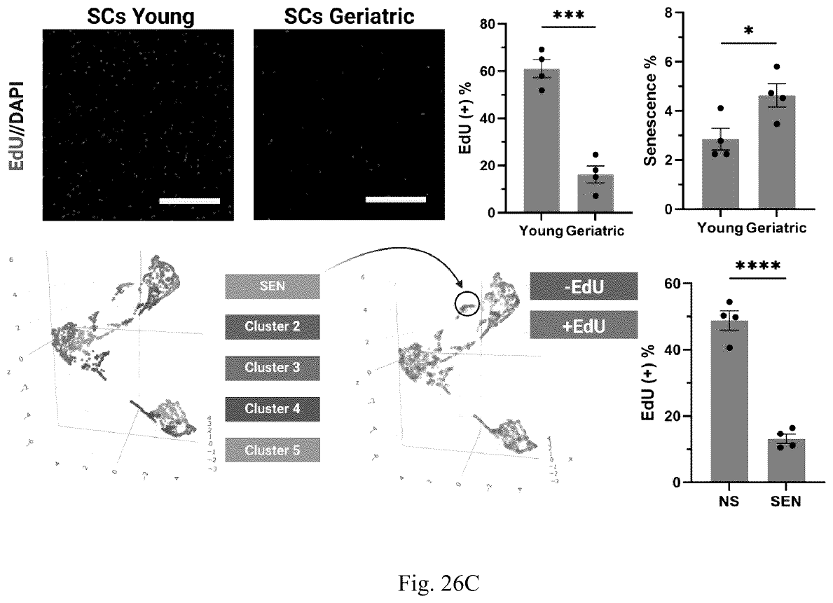

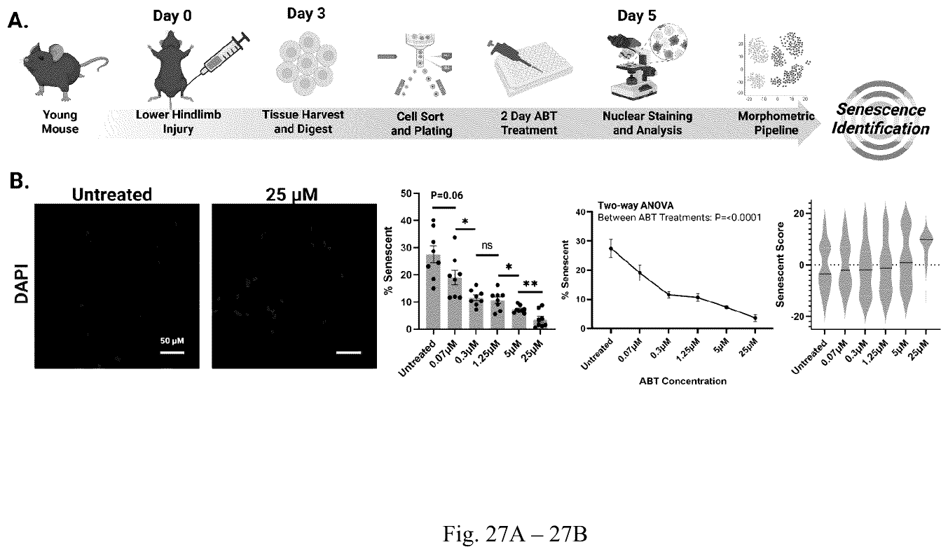

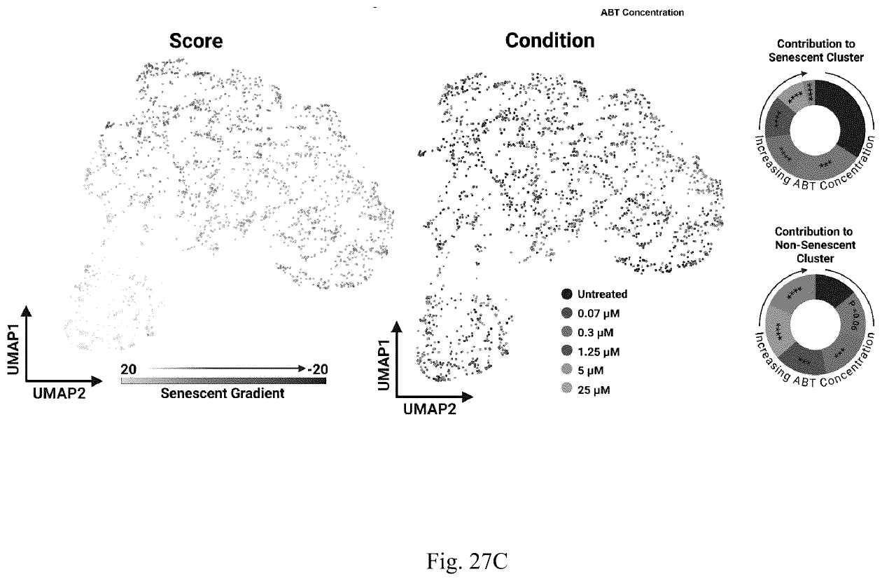

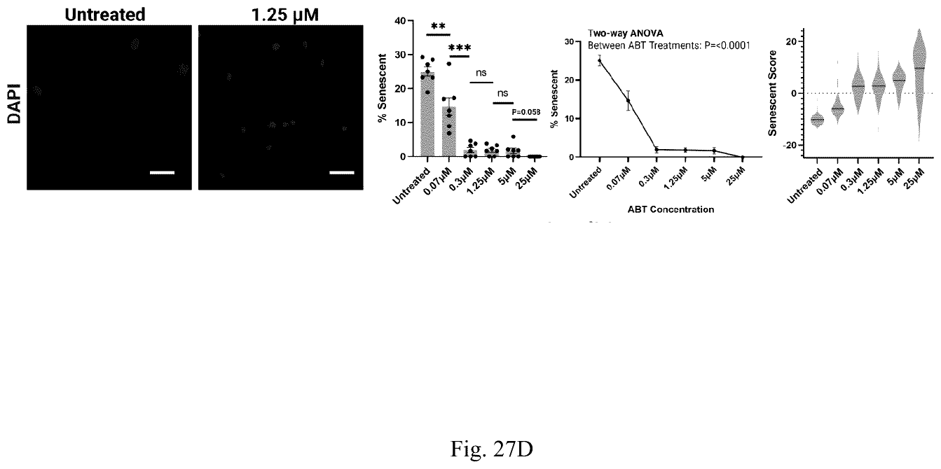

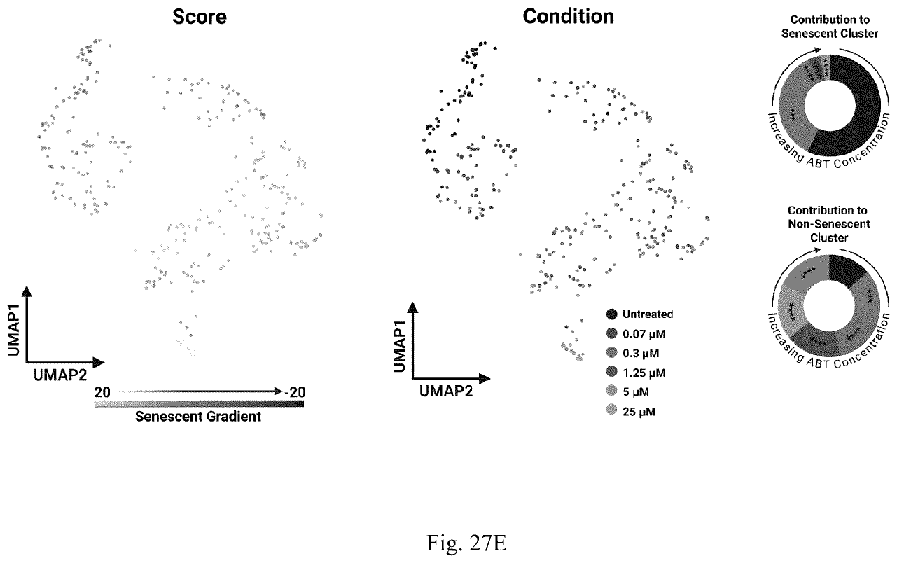

Furthermore, the loss of proliferative capacity in geriatric cells (decreased EdU incorporation) was correlated with the NMP-identified SnCs, focusing on FAPs and SCs due to their aforementioned dynamic senescence across aging (FIG. 26A). A 56% and 73% reduction in EdU in FAPs and SCs, respectively, was detected when comparing cells from geriatric and young mice that were grown in culture (FIG. 26B, FIG. 26C). The cells identified as the SnC population by the NMP proved to have significantly reduced EdU positivity, confirming an exit from the cell cycle (FIG. 26B, FIG. 26C). In addition, senolytic treatment was applied to SCs and FAPs from injured tissue to confirm the ability of the NMP to identify SnCs ex vivo (FIG. 27A). For both FAPs (FIG. 27B, FIG. 27C) and SCs (FIG. 27D, FIG. 27E), the NMP detected the dose-dependent decrease in senescent nuclei with increasing concentrations of ABT-263. SCs were seemingly more sensitive to treatments and began displaying signs of toxicity at higher concentrations. These data support the capabilities of the NMP to accurately identify SnCs in sorted primary cells.

The NMP Maintains Efficacy with Alternative Tissue Sources

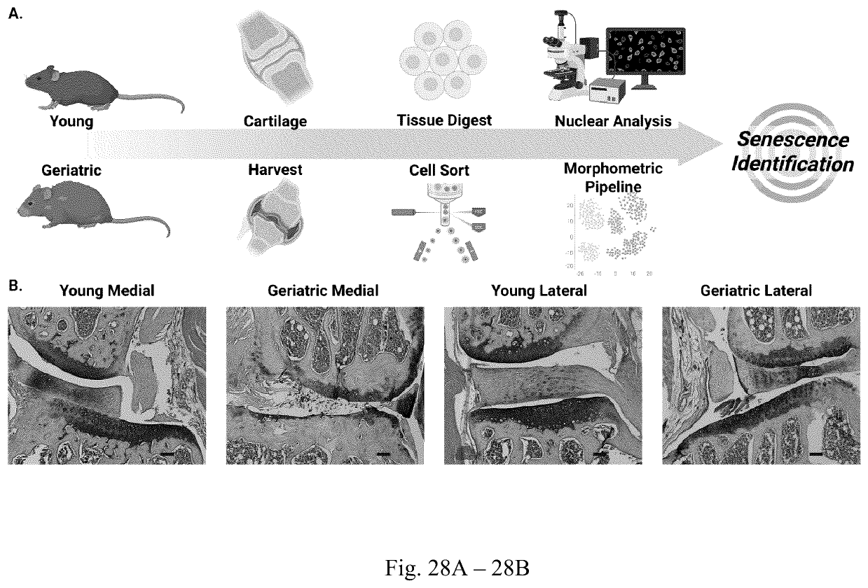

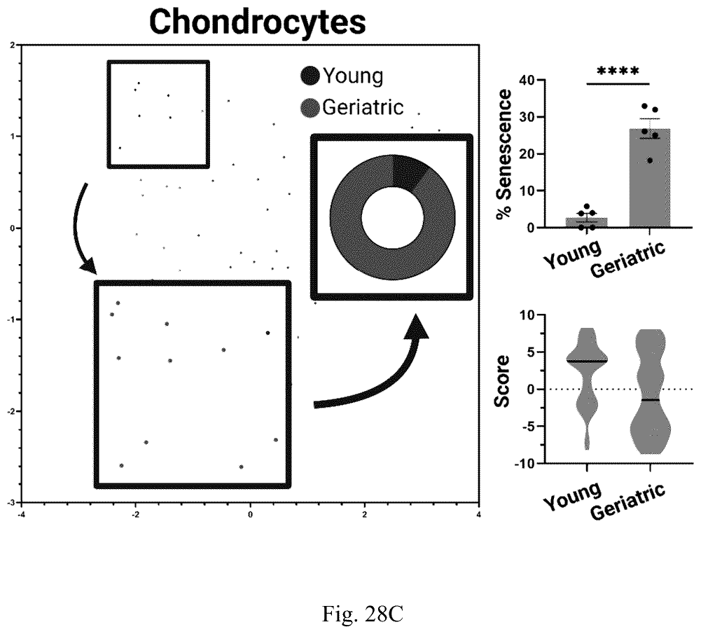

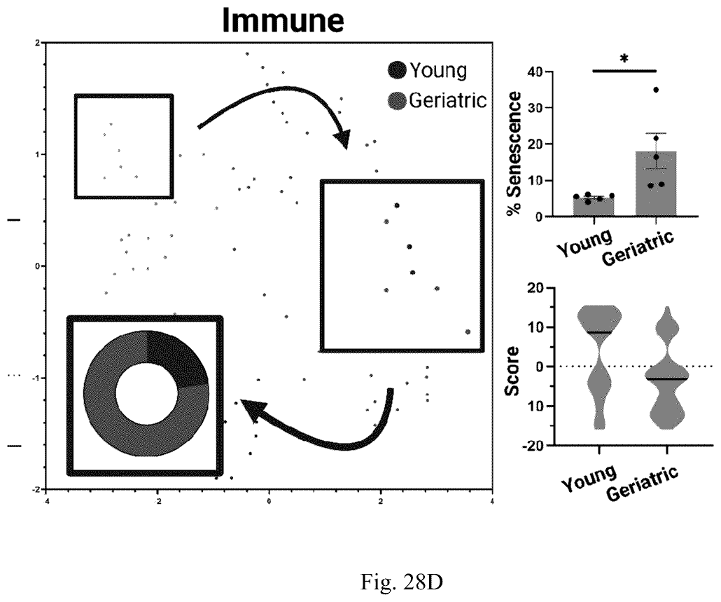

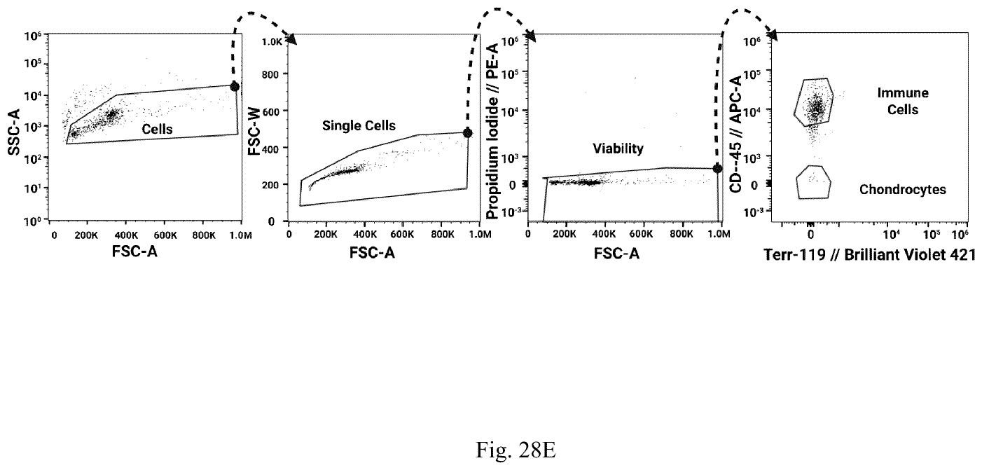

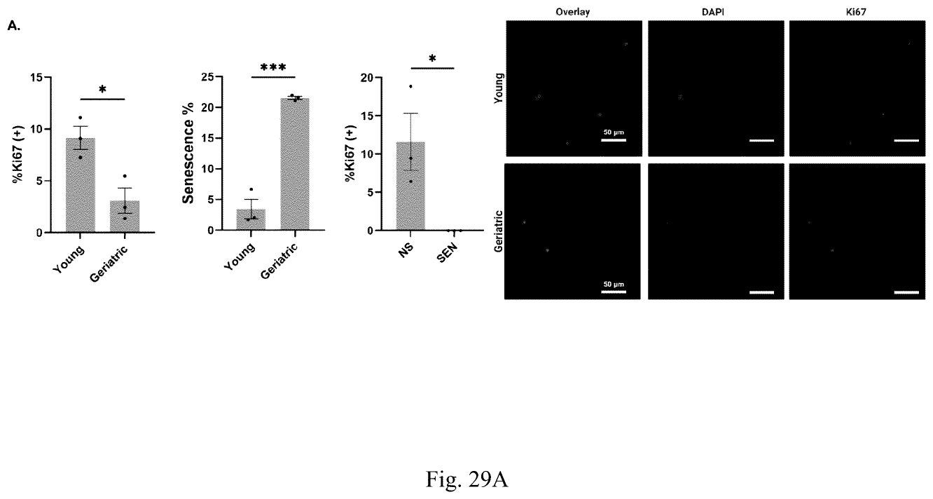

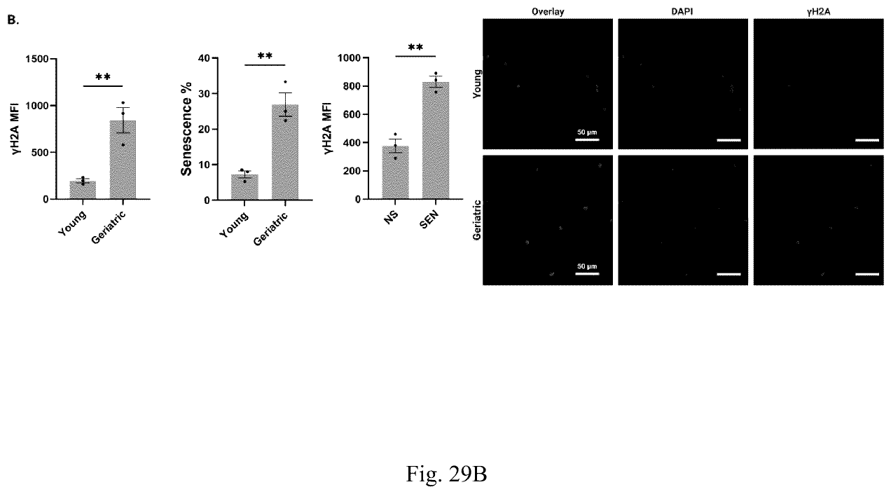

Finally, to test if the NMP can be applied to additional aged-related degenerative disorders associated with SnC load, the disclosed pipeline was used to evaluate chondrocytes in primary osteoarthritis in aging mice (FIG. 28A) (Coryell, P. R., et al., Nat. Rev. Rheumatol., 2021). Degenerative articular cartilage from geriatric mice was compared to its healthy counterpart from young mice (FIG. 28B). A similar approach to the above SkM process was implemented to analyze FACS-isolated cells enriched for chondrocytes and ICs (FIG. 28E). A robust senescent signature was revealed in chondrocytes from geriatric cartilage that represented about a 10-fold increase in SnCs compared to chondrocytes from young tissue (FIG. 28C). This was corroborated with decreasing Ki67 and increasing γH2A expression from sorted chondrocytes (FIG. 29A, FIG. 29B). ICs from geriatric mice also showed an increase in SnCs compared to young counterparts, with about a 3-fold increase (FIG. 28D). These data support the current paradigm that chondrocytes of arthritic milieus are senescent and demonstrate the utility of the disclosed NMP as a robust means to identify SnCs from primary tissue.

### Discussion

The disclosed study establishes an approach that quantifies and reduces nuclear morphometrics to reliably identify SnCs, thereby circumventing many of the problematic assays currently used (FIG. 10A-FIG. 10E). The ease of use of the NMP, along with its consistency, provides a means of greater standardization for SnC identification. NMP-derived scores facilitate comparisons of nuclear morphometrics within a single UMAP, while clusters enable comparisons between independent UMAPs. With this comes a better understanding of the biology of SnCs in endogenous processes, such as regeneration and aging, ultimately helping define better targeting of these cells for therapeutics.

Recent reports of alternative learning approaches using nuclear morphologies have also demonstrated promising results in specific biological contexts, recognizing the relationship of unique nuclear changes with senescence conversion (Heckenbach, I. et al., Nat. Aging, 2022; and Duran, I. et al., Nat. Commun., 2024). Most of these systems used supervised learning to train a system on datasets of nuclei that are enriched for SnCs. While these potentially can reveal alternative morphologies associated with the senescence fate choice, these measures may be limited to the training dataset. The disclosed approach uses unsupervised learning, where the nuclear measures were chosen for broad applicability, capable of accurately identifying SnCs across mechanisms of senescence, cell types, and tissue types. Clustering and dimensional reduction were biologically confirmed and interpreted in a semi-supervised context. The efficacy of the disclosed NMP is demonstrated by its ability to detect SnCs in culture, in freshly sorted cells, and in tissue sections, environments that all have differing mechanical influences on cells and their nuclei. Only a minor number of nuclei that express proliferative markers are ever detected in senescent clusters. These data acknowledge the conserved nature of the underlying morphometrics in the NMP for the senescent phenotype.

The nuclear morphometrics used for the NMP were specifically identified for their direct relationship to cell cycle dynamics and senescence, being a phenotype of permanent exit in the G1 or G2 phase (Roger, L., et al., Int. J. Mol. Sci., 2021). In fact, nuclear size and DAPI intensity have been connected to cell cycle phases (Ferro, A. et al. Lab. Investig., 2017). These characteristics are associated with senescence, presumably due to the sustained p53 expression and subsequent deregulation of mTOR (Manohar, S. et al., Mol. Cell, 2023; and Maskey, R. S. et al., Cell Cycle, 2021), and the formation of actin stress fibers, causing nuclear flattening in SnCs. In addition, nuclear blebbing leading to decreased circularity in SnCs may be a result of Lamin B1 degradation (Matias, I. et al., Aging Cell, 2022; and Romanov, V. S. et al., Cell Cycle, 2010), and heterochromatic foci appearing as punctate nuclear densities in SnCs are associated with highly methylated DNA regions caused by the increased expression of cycle arrest regulators, such as E2F (Pospelova, T. V., Methods Mol. Biol., 2013; and Narita, M. et al., Cell, 2003). The disclosed system demonstrates a minimum criterion of four morphological changes (increase in nuclear size and dense foci, decrease in nuclear circularity and DAPI intensity) to unequivocally identify bona fide SnCs (FIG. 12A-FIG. 12E). Each of the morphometrics can independently present as a cell begins to acquire damage and initiate its path to senescence, as demonstrated by the visualized “gradient” in the disclosed NMP results (FIG. 12B). With the current clustering scheme, the population labeled as senescent-like may indicate a pre-senescent nucleus, where this morphological phenotype indicates a cell-fate determination step or a precursor to full-fledged senescence. This trajectory of senescence reveals an interesting concept that could be used for next-generation senolytics, derived to target cells earlier in the transition, potentially improving the limited efficacy of senescence therapies (Lane, N. et al., Osteoarthr. Cartil., 2021).

The application of the disclosed NMP in SkM homeostasis and regeneration in young, aged, and geriatric mice revealed minimal SnCs in homeostatic tissue, but a robust SnC expansion following injury in all ages (FIG. 19). The regenerative SnC expansion in young mice was mainly composed of FAPs at a time coinciding with their peak proliferative expansion (Wosczyna, M. N. et al., Cell Rep., 2019), supporting previous reports of their necessity for efficient regeneration potentially through the SASP-dependent regulation of the immune cell response (Chikenji, T. S. et al., EBioMedicine, 2019). Interestingly, FAPs from aged uninjured tissue grown in culture for 3 days were more senescent than those from young tissue, indicating aged cells are sensitive to replicative senescence, while young cells may be more responsive to stress-induced senescence of the in vivo regenerative milieu (compare FIG. 26B with FIG. 19C and FIG. 19D). However, regeneration in aged environments have altered cellular compositions of SnCs, with SCs and Enr-ECs becoming the foremost components and FAPs falling to a minority. These data are consistent with a detrimental role of SC senescence in aged SkM, as this has been shown to limit the stem cell pool that directly contributes cells for the rebuilding muscle fibers (Zeng, W. et al., Dev. Cell, 2023; and Sousa-Victor, P. et al., Nature, 2014) and the unfavorable inflammatory profile of senescent ECs, as a potential source of chronic inflammation in aging (Grosse, L. et al., Cell Metab, 2020). While the disclosed data does not show a drastic senescence shift in ICs from young to geriatric age, the presence of immune SnCs can have a compounding detrimental effect on their inherent ability to clear SnCs (Lee, K.-A., et al., Front. Aging, 2022). In addition, the NMP was translated to the assessment of SnCs in tissue sections (FIG. 22A-FIG. 22F, FIG. 23A-FIG. 23F, FIG. 24, and FIG. 25), supporting its broad use across cellular environments to detect cells with senescent characteristics. These data also reproduced the dynamic, age-dependent changes in SnC populations relative to FAPs and ECs, minimizing any impact of tissue processing and ex vivo treatments on cellular representations. Further use of the disclosed NMP in age-associated primary osteoarthritis in mice clearly defines senescent chondrocytes in geriatric articular cartilage (FIG. 28A-FIG. 28E). While technical challenges of chondrocyte isolation limit the number of cells isolated per replicate, the NMP was still capable of achieving reliable and significant results in this context. Similar to SCs in SkM, senescence in chondrocytes contributes to joint pathology by reducing their functional capacity (decreased ECM production) and likely by promoting a chronic inflammatory environment through SASP expression, which negatively impacts non-SnCs (Yagi, M., et al., Sci. Rep., 2023).

Collectively, these data demonstrate that the disclosed NMP can be implemented in different environments across ages and translated to many tissues to define SnCs. The intriguing dynamic representations of SnCs revealed by using the NMP reinforce the importance of considering the age of an individual when attempting to clear SnCs with an approach that does not discriminate between their different origins, as one may be beneficial and the other detrimental.

There is a clear need for new methodologies to identify senescence. While the disclosed study sought to establish a morphological platform that maintained its sensitivity and efficacy across environments, further testing in various tissues, especially those of human origin, will be important. Furthermore, the challenge in identifying cellular senescence lies in the heterogeneity of the phenotype, which has limited the consistent use of markers across conditions. As such, a multiparameter approach to identify senescence can improve accuracy through the use of platforms like the disclosed NMP, and the identification of alternative markers that are identified. While using potentially nonspecific markers to validate new methods remains a challenge, correlating multiple biomarkers with SnC detection, as employed here, can increase confidence in reliably identifying true senescent states.

### Methods

### Animal Models

Animal experiments were in accordance with the Institutional Animal Care and Use Committee (IACUC, protocols 202100001/2). All mice were on a C57BL/6 J background and acquired from the colony at the National Institute on Aging of the National Institutes of Health. Ages ranged from 3-6 months for young, 23-25 months for aged, and 28-31 months for geriatric mice. All mice were male to accommodate sufficient numbers in aged cohorts, and were maintained in 12 h/12 h light/dark conditions in a controlled environment held at 21.2° C., and 58% humidity.

### Cell Culture and Staining

Cells were cultured according to ATCC, growth media (C2GM, 10% FBS, 1× Penicillin/Streptomycin, and DMEM. C2C12s were used between passages 5 and 25. To induce senescence, C2C12 cells were plated between 1666-5000 cells/cm² on day 1. On day 2, cells were treated with 300 or 500 μM of H₂O₂, 2 or 10 μM of Etoposide, or 0.08 or 0.4 μM of Doxorubicin for 2 h, treatment was removed, and fresh C2GM was added to cells that were cultured for an additional 3 days and then fixed with 4% PFA. For ABT-263 treatment, the same protocol was followed, and on day 5, cells were treated with a dose-response of ABT-263 for 24 h before fixing.

3T3-L1 cells were acquired from ATCC and cultured accordingly, growth media contained 10% calf serum and 1× Penicillin/Streptomycin, in DMEM. 3T3-L1 were used between passages 3 and 25. To induce senescence, 3T3-L1 cells were plated between 833-2000 cells/cm² on day 1. On day 2, cells were treated with 150 or 300 μM of H₂O₂ for 4 h; or 1 or 3 μM of Etoposide, or 0.03 or 0.1 μM of Doxorubicin overnight (16 hrs); treatment was removed, and fresh 3TGM was added to cells that were cultured for an additional 2-3 days and then fixed with 4% PFA.

Senescence was detected with the Senescence β-Galactosidase Staining Kit according to the manufacturer's protocol, with pH adjusted to 6.0. Brightfield and fluorescent images were captured on the Keyence BZ-X810 microscope. DAPI-stained and overlaid substrate-positive (blue) cells were quantified as senescent.

For immunostaining, cells were fixed with 4% paraformaldehyde (PFA) for 10 min, rinsed in 1×PBS, permeabilized with 0.1% TritonX for 30 min, and blocked with 1% BSA/10% DS/1×PBS/0.1% tween for 1.5 h. Following this, the cells were incubated with primary antibodies and diluent (1% BSA/10% DS/1×PBS) for 18-20 h. Antibodies included: γH2A.X, Ki67, Cleaved-Caspase-3. After washing with 1×PBS to remove the primary antibodies, cells were incubated with diluent and secondary antibodies for 2 h. Secondary antibodies included: Alexa Fluor 555 Donkey anti-Rabbit. Cells were then washed with 1×PBS and counterstained with DAPI. All samples were maintained at 4° C. until imaging with the Nikon Eclipse Ti2-LAPP Inverted Microscope System. Images were taken at 20× with 1 to 5 images taken per well. Images were then quantified with NIS-Elements AR Analysis Software, for positivity of fluorescence and/or intensities.

### RT-qPCR

Total RNA extraction from cultured cells was performed according to the manufacturer's protocol with the RNeasy Micro or Plus Mini Kit. RNA quantitation was assessed on the Thermo Scientific NanoDrop One, and cDNA was generated with the RevertAid RT Reverse Transcription Kit. The mRNA expression was determined on a QuantStudio5 Real-Time PCR System using FastStart SYBR Green Master. Relative expression levels were calculated with the 2^{-ΔΔCT} method using GAPDH as the reference gene, and each independent experimental replicate included 2-3 technical replicates. Gene expression noted as undetectable by the instrument was assigned a value of 40 Ct. Primer sequences are in Table 2 below and were used at 0.2 μM concentration.

TABLE 2

| GenBank | PrimerBank | Primer | Forward Sequence | Reverse Sequence |
|---|---|---|---|---|
| Accession | ID | Name | (5′-3′) | (5′-3′) |
| NM_011333 | 141803162c1 | mCCL2 | TAAAAACCTGGATCGG | GCATTAGCTTCAGATT |
| | | | AACCAAA | TACGGGT |
| | | | (SEQ ID NO: 1) | (SEQ ID NO: 2) |
| NM_001111099 | 6671726a1 | mCDKN1A | CCTGGTGATGTCCGAC | CCATGAGCGCATCGC |
| | | | CTG | AATC |
| | | | (SEQ ID NO: 3) | (SEQ ID NO: 4) |
| NM_013693 | 133892368c1 | mTNF | CAGGCGGTGCCTATGT | CGATCACCCCGAAGT |
| | | | CTC | TCAGTAG |
| | | | (SEQ ID NO: 5) | (SEQ ID NO: 6) |
| NM_001037722 | 109148516c2 | mADAM15 | ATGGCACCCGAATGGT | CTCCAGTGTATAGCCT |
| | | | CAG | CTCTCTG |
| | | | (SEQ ID NO: 7) | (SEQ ID NO: 8) |
| NM_031168 | 13624310c1 | mIL6 | CTGCAAGAGACTTCC | AGTGGTATAGACAGG |
| | | | ATCCAG | TCTGTTGG |
| | | | (SEQ ID NO: 9) | (SEQ ID NO: 10) |
| NM_001164197 | 255958311c3 | mMMP19 | CCTGGTCCCATGCCAA | CCCTTGAAAGCATAA |
| | | | ACC | GTCTTCCC |
| | | | (SEQ ID NO: 11) | (SEQ ID NO: 12) |
| NM_011577 | 6755774c1 | mTGFβ1 | CCACCTGCAAGACCAT | CTGGCGAGCCTTAGT |
| | | | CGAC | TTGGAC |
| | | | (SEQ ID NO: 13) | (SEQ ID NO: 14) |
| NM_010721 | 188219588c2 | mLMNB1 | GAGTATGAGGCGGCA | CATCTGCTAACTGCT |
| | | | CTAAAC | TTTTGGC |
| | | | (SEQ ID NO: 15) | (SEQ ID NO: 16) |
| NM_008610 | NA | mMMP2 | TGCAGGAGACAAGTT | GACGGCATCCAGGTT |
| | | | CTGGA | ATCAG |
| | | | (SEQ ID NO: 17) | (SEQ ID NO: 18) |
| NM_008084 | NA | mGAPDH | TCAAGAAGGTGGTGA | GTTGAAGTCGCAGGA |
| | | | AGCAG | GACAA |
| | | | (SEQ ID NO: 19) | (SEQ ID NO: 20) |

### Muscle Injury

Mice were anesthetized with isoflurane, and injury was induced with 90 ul of 1.2% BaCl₂ injected intramuscularly into the lower hindlimb muscles, which were isolated at the days post injury (DPI) noted in the main text. The gastrocnemius was used to isolate cells from injured SkM, and the tibialis anterior (TA) were isolated for histological analysis.

### Cell Isolation and Fluorescence Activated Cell Sorting

To isolate cells from muscle tissue, dissected tissue was minced with scissors and then incubated in 760 U/mL collagenase type 2 in Ham's F10 for ˜1 h in a 37° C. shaking water bath, then washed with wash media (WM, 10% Horse Serum (HS) in Ham's F-10), and digested again in a 1 to 8.5 dilution of both 1000 U/ml collagenase type 2 and 11 U/ml dispase in WM for 30 min in a 37° C. shaking water bath. The samples were triturated seven times with a syringe fixed with a 21-gauge needle, diluted in wash media, and then passed through a 40 μm strainer. The single-cell suspension was stained with fluorescent-conjugated antibodies for 30 min at 4° C. Fluorescence-conjugated antibodies included CD-45, Sca-1, VCAM, CD-31. Cells were then passed through 35 μm blue-top filters and sorted using the Sony MA900 Multi-Application Cell Sorter Software for an enriched fraction of endothelial cells (Enr-ECs), immune cells (ICs), fibroadipogenic progenitors (FAPs), and satellite cells (SCs) (gating schemes in FIG. 20H) (Quarta, M. et al., Nat. Commun., 2017; Ieronimakis, N., PLoS ONE, 2008; Liu, L., et al., Nat. Protoc., 2015; and Wosczyna, M. N. et al., Cell Stem Cell, 2021). Cells were plated in ECM-coated 96-well plates containing WM, centrifuged at 500×g for two minutes, and incubated overnight to allow for adherence. After 12 h, cells were fixed with 4% PFA for 10 min and washed with 1×PBS. The cells were then stored at 4° C. until imaging.

For EdU staining, cells were sorted using the Sony MA900, plated on ECM-coated 96-well plates containing growth media ((GM), 10% HS, 20% FBS, 1:100 Penicillin/Streptomycin, and 2.5 ng/ml FGF in DMEM), then incubated for 3 days (SCs or FAPs) with a pulse of EdU (5 μM) five hours prior to fixing with 4% PFA for 10 min and washed with 1×PBS. EdU incorporation was detected with the Alexa Fluor 555 Click-iT™ Plus EdU Cell Proliferation Kit using the manufacturer's recommended protocol and stored at 4° C. until imaging.

For ABT-263 treatment of FACS isolated FAPs and SCs, 3 DPI muscle tissue from two 5 month old mice were combined and this was done twice to make two input samples. Each sample was sorted using the Sony MA900, plated on ECM-coated 96-well plates containing wash media (WM; 10% HS in Ham's F-10) at a cellular density of 5000 cells per well. Each sample was divided into four replicates. After incubating for 12 h, cells were treated with a dose-response of ABT-263 as noted in FIG. 27 for 48 h before fixing.

To isolate chondrocytes from the knee joints, dissected cartilage was collected using a scalpel as previously described, except for minimizing endochondral bone carryover (McCool, J. L., et al., Methods Mol. Biol., 2022). The cartilage was then incubated in 760 U/mL collagenase type 2 in Ham's for about 2 h at 37° C. shaking water bath. The samples were then passed through a 40 μm filter. The single-cell suspension was stained with fluorescent-conjugated antibodies for 15 min at 4° C. Fluorescent antibodies included Terr-119 and CD-45. Cells were then stained with Propidium Iodide, passed through blue-top filters, and sorted using the Sony MA900 Multi-Application Cell Sorter (FIG. 28E). Cells were plated on ECM-coated 96-well plates in WM, centrifuged at 500×g for 2 min, incubated overnight, and fixed 12 h later with 4% PFA for 10 min. All samples were maintained at 4° C. until imaging.

To detect senescence-associated β-galactosidase, the FastCellular Senescence Detection Kit was used prior to sorting according to the manufacturer's protocol. Briefly, samples were incubated at 37° C. in Bafilomycin A1 for 1 h, stained for 30 min at 37° C. with SPIDER-β-gal/Bafilomycin A1, resuspended in WM and maintained at 4° C. until analyzed on the Sony MA900 (FIG. 10C).

### Skeletal Muscle Histology and Immunofluorescence

Tibialis anterior (TA) muscles, injured 3 days prior, were isolated on the tibia and fixed in 0.5% paraformaldehyde solution at 4° C. for 5 days. Tissues were then washed with 1×PBS overnight, followed by detachment from the tibia and incubation in 30% sucrose overnight, both at 4° C. The TA tissues were then suspended in Tissue-Plus O.C.T. Compound and frozen in dry ice-cooled isopentane. Cryosections were generated on a Leica cryostat at a thickness of 12 μm, allowed to dry for 30 min, and stored at −80° C.

For immunocytochemical analysis, cryosections were rehydrated in 1×PBS, permeabilized in 0.1% Triton-X for 20 min, blocked in diluent (1% BSA/10% DS/1×PBS) with 0.1% tween for 2 h, then incubated with primary antibodies in diluent for 18-20 h at 4° C. Primary antibodies consisted of laminin, γH2AX, Ki67, CD31, and PDGFRα. Cryosections were then washed with 1×PBS and incubated with secondary AlexaFluor conjugated antibodies in diluent for 2 h. Finally, samples were washed with 1×PBS, counterstained with DAPI, and coverslip mounted with FluoroGel medium.

### Knee Joint Processing for Cartilage Histology

Samples were fixed in 10% buffered formalin for 72 h and decalcified in 10% formic acid for 10 days. Decalcified samples were embedded in paraffin, and 4 μm sections were cut perpendicular to the cartilage surface. Sections were stained with Safranin O/Fast green using routine methods. Cartilage destruction in the mice was examined using Safranin O staining.

### Fluorescence Imaging

Fluorescent images were acquired using the Nikon Eclipse Ti2-LAPP Inverted Microscope System equipped with the NIS-Elements Advanced Research acquisition software. Three representative high-magnification images were acquired with a 20× objective per well. For cryosectioned samples, 3-5 representative high-magnification images were acquired with a 60× oil immersion objective. For EdU-stained samples, the entire well was imaged. Z-stacked Images were taken in 1 μm increments. Exposures and look-up tables (LUTs) were held constant for each independent experiment.

### Pre-Processing Pipeline

Image pre-processing was performed with NIS-Elements Advanced Research (AR) Analysis Software. Initial preprocessing started with a 3D deconvolution to remove noise from a Z-stacked image. Then, a max intensity projection was used to allow for all fluorescence to appear on a single plane and ease the analysis of the image. To further refine the images, a low-pass filter was used to pass only details larger than a set pixel value and remove small irregularities. Finally, a rolling ball function was used to further differentiate background and fluorescence intensities.

### Nuclei Phenotypic Extraction

Image analysis was performed with the NIS-Elements AR Analysis Software to achieve single-nuclei resolution. To detect nuclei, a threshold function set for each sample was used on images of wells stained with DAPI, allowing the formation of binary objects in the image. Limitations were made on size and circularity to remove objects such as debris or overlapping nuclei. By creating the binary image, nuclei could be separated, counted, and measured for size, mean intensity, and circularity. To detect foci, a spot detection function was used to count the number of bright circular objects that contrasted from surrounding pixels. Limitations on spots included size, contrast, and intensity. These spots were then aggregated to only count bright spots that were present in the binary data of the detected nuclei. For C2C12 cells/3T3-L1s, as well as primary FAPs and SCs, analyses were automated through the software on whole images. For primary endothelial and immune cells, analyses were done both on whole images and manually to avoid misidentification due to cell aggregation.

### UMAP Presentation and Clustering

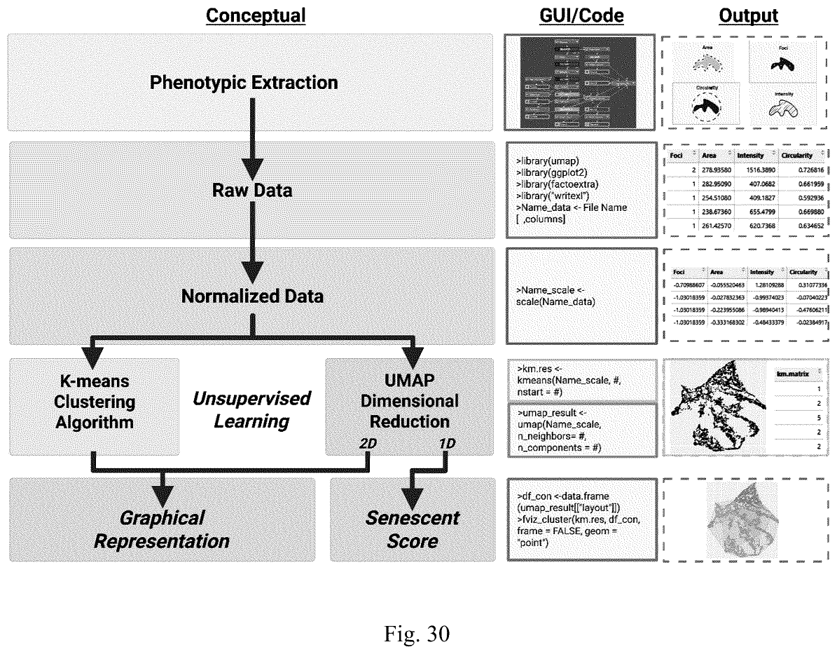

Using R Studio, the four phenotypic measures of nuclei were imported and normalized using a z-score within their respective parameters. To cluster visually similar cells, package factoextra was used, specifically the function fviz_cluster, which applies a k-means algorithm on the 4 normalized phenotypic measures. Using the R studio package UMAP, a two-dimensional visualization (n_neighbors=20, n_components=2) of the four parameters was made to localize visually similar cells. To form the senescence score, the prior step was repeated with n_components=1 and required a high number of cells to achieve optimal dynamic range. For three-dimensional visualization, the prior step was repeated with n_components=3. The clustering data, which is done at the higher dimension, is then visualized on the lower-dimensional UMAP. Within each UMAP, conditions are equally represented by inputting an equal number of cells from each condition into the platform, ensuring accurate quantification. Data was exported and graphed with GraphPad Prism. See FIG. 30 for a diagrammatic overview of these processes.

### Statistics and Reproducibility

All reported measurements were obtained from distinct experimental samples. Experiments were performed at least three times, using a single biological/experimental (not technical) replicate per condition in each experiment. “n” numbers indicate independent experiments unless otherwise noted. Statistical analyses were performed using GraphPad Prism. Error bars represent mean±standard error of the mean (SEM). Statistical tests were conducted using two-sided/tests unless otherwise specified. Significance is indicated as follows: p>0.05 (n.s.—not significant); p≤0.05 (*); p≤0.01 (**); p≤0.001 (***); p≤0.0001 (****). No data were excluded from analysis except for one replicate of RT-qPCR due to a technical issue. No statistical method was used to predetermine sample size. Experiments were randomized, and investigators were not blinded to sample allocation or condition during experiments or during immuno-fluorescence image analysis.

FIG. 10B includes n=3 experimental replicates, with per-condition cell counts as follows: X-Gal, 100-300 cells; Ki-67, 100-300 cells; γH2A.X, 150-250 cells; and cleaved caspase, 100-600 cells; FIG. 10C includes n=3 experimental replicates, with 400-4000 SA-β-Gal cells per condition; FIG. 10D includes n=4 experimental replicates, each with two to three technical replicates; FIG. 10E includes n=3 experimental replicates, with 4000-8000 cells per replicate; FIG. 15E and FIG. 15F include n=3 experimental replicates, with 1000-1500 cells per replicate; FIG. 19B-FIG. 19 E include n=3 biological replicates, with cell counts per replicate as follows: FAPs, 1.5-5.0×10³ cells; SCs, 1.0-1.2×10³ cells; FIG. 22B and FIG. 22C include n=3 biological replicates, with 150-200 cells per condition; FIG. 22E include n=3 biological replicates, with 300-600 cells per condition; FIG. 16A-FIG. 16L includes n=3 experimental replicates per inducer with X-Gal n=100-600 cells, γH2A n=100-500 cells, and Ki-67=200-350 cells per condition; FIG. 16A-FIG. 16L include n=3 experimental replicates per inducer with n=800-1100 cells per condition with γH2A n=250-600 cells, and Ki-67 n=150-600 cells per condition; FIG. 20A-FIG. 20F include n=3 biological replicates, with cell counts per replicate as follows: FAPs, 200 cells; SCs, 200 cells; FIG. 23C and FIG. 17C include n=3 biological replicates, with 100-300 per condition; FIG. 23F and FIG. 17F include n=3 biological replicates, with 200-600 cells condition; FIG. 26B includes n=4 biological replicates, with 1000-2000 cells per condition; FIG. 26C includes n=4 biological replicates, with 500-1500 cells per condition; FIG. 27C includes n=8 experimental replicates with cell input pulled from 2 mice to make 4 replicates. This was done twice; FIG. 27E includes n=7 experimental replicates with cell input pulled from 2 mice to make 4 replicates, and 2 mice to make 3 replicates. FIG. 28C and FIG. 28D include n=5 biological replicates, with n=82-96 cells per condition, n=123-134 cells per condition, for chondrocytes and immune cells, respectively.

### Figure Construction

Figures were assembled using BioRender, and schematics were created in BioRender where noted in the figure legends. Graphs were made using GraphPad Prism unless otherwise stated. FIG. 12A was made using the Rstudio package ggplot2 and extension ggally. FIG. 12C was made using the Rstudio package kohonen: supervised and unsupervised self-organizing maps. Three-dimensional UMAPs were made using the Rstudio package plotly. FACS plots were constructed using FlowJo. The p-values for the graphs included in the figures are in Table 3 below.

TABLE 3

| FIG. // Panel | Condition | Comparison | | | | | P-Value |
|---|---|---|---|---|---|---|---|
| 10B | X-Gal | Untreated | | v | 300 | μm | 0.000688 |
| 10B | X-Gal | 300 | μm | v | 500 | μm | 0.052366 |
| 10B | X-Gal | Untreated | | v | 500 | μm | 0.001696 |
| 10B | γH2A | Untreated | | v | 300 | μm | 0.009585 |
| 10B | γH2A | 300 | μm | v | 500 | μm | 0.03013 |
| 10B | γH2A | Untreated | | v | 500 | μm | 0.000563 |
| 10B | Ki67 | Untreated | | v | 300 | μm | 0.00622 |
| 10B | Ki67 | 300 | μm | v | 500 | μm | 0.00481 |
| 10B | Ki67 | Untreated | | v | 500 | μm | 0.000877 |
| 10B | Caspase | Untreated | | v | 300 | μm | 0.325025 |
| 10B | Caspase | 300 | μm | v | 500 | μm | 0.442187 |
| 10B | Caspase | Untreated | | v | 500 | μm | 0.889914 |
| 10C | Spider-Gal | Untreated | | v | 300 | μm | 0.02831 |
| 10C | Spider-Gal | 300 | μm | v | 500 | μm | 0.054592 |
| 10C | Spider-Gal | Untreated | | v | 500 | μm | 0.030466 |
| 10E | Foci | Untreated | | v | 300 | μm | 0.027277 |
| 10E | Foci | 300 | μm | v | 500 | μm | 0.005821 |
| 10E | Foci | Untreated | | v | 500 | μm | 0.001825 |
| 10E | Area | Untreated | | v | 300 | μm | 0.002788 |
| 10E | Area | 300 | μm | v | 500 | μm | 0.018119 |
| 10E | Area | Untreated | | v | 500 | μm | 0.000769 |
| 10E | Intensity | Untreated | | v | 300 | μm | 2.38E−07 |
| 10E | Intensity | 300 | μm | v | 500 | μm | 0.000222 |
| 10E | Intensity | Untreated | | v | 500 | μm | 1.84E−05 |
| 10E | Circularity | Untreated | | v | 300 | μm | 0.000126 |
| 10E | Circularity | 300 | μm | v | 500 | μm | 0.001389 |
| 10E | Circularity | Untreated | | v | 500 | μm | 0.000406 |
| 12C | Senescent % | Untreated | | v | 300 | μm | 0.005439 |
| 12C | Senescent % | 300 | μm | v | 500 | μm | 0.002679 |
| 12C | Senescent % | Untreated | | v | 500 | μm | 4.83E−05 |
| 12D | X-Gal | NS | | v | SEN | | 0.01191 |
| 12D | Ki67 | NS | | v | SEN | | 0.003782 |
| 12D | γH2A | NS | | v | SEN | | 0.001133 |
| 15A | X-Gal | Untreated | | v | 2 | μm | 0.002365 |
| 15A | X-Gal | 2 | μm | v | 10 | μm | 0.002538 |
| 15A | X-Gal | Untreated | | v | 10 | μm | 0.000584 |
| 15A | γH2A | Untreated | | v | 2 | μm | 0.001449 |
| 15A | γH2A | 2 | μm | v | 10 | μm | 0.000492 |
| 15A | γH2A | Untreated | | v | 10 | μm | 0.00028 |
| 15A | Ki67 | Untreated | | v | 2 | μm | 0.004924 |
| 15A | Ki67 | 2 | μm | v | 10 | μm | 0.01475 |
| 15A | Ki67 | Untreated | | v | 10 | μm | 0.001506 |
| 15B | X-Gal | Untreated | | v | 0.08 | μm | 0.017562 |
| 15B | X-Gal | 0.08 | μm | v | 0.4 | μm | 0.005774 |
| 15B | X-Gal | Untreated | | v | 0.4 | μm | 0.000128 |
| 15B | γH2A | Untreated | | v | 0.08 | μm | 0.005597 |
| 15B | γH2A | 0.08 | μm | v | 0.4 | μm | 0.181026 |
| 15B | γH2A | Untreated | | v | 0.4 | μm | 0.000689 |
| 15B | Ki67 | Untreated | | v | 0.08 | μm | 0.001781 |
| 15B | Ki67 | 0.08 | μm | v | 0.4 | μm | 0.028094 |
| 15B | Ki67 | Untreated | | v | 0.4 | μm | 0.000433 |
| 15C | Foci | Untreated | | v | 2 | μm | 0.016866 |
| 15C | Foci | 2 | μm | v | 10 | μm | 0.06445 |
| 15C | Foci | Untreated | | v | 10 | μm | 0.004778 |
| 15C | Area | Untreated | | v | 2 | μm | 0.001634 |
| 15C | Area | 2 | μm | v | 10 | μm | 0.003505 |
| 15C | Area | Untreated | | v | 10 | μm | 0.000211 |
| 15C | Intensity | Untreated | | v | 2 | μm | 3.46E−07 |
| 15C | Intensity | 2 | μm | v | 10 | μm | 0.000573 |
| 15C | Intensity | Untreated | | v | 10 | μm | 2.02E−08 |
| 15C | Circularity | Untreated | | v | 2 | μm | 0.014949 |
| 15C | Circularity | 2 | μm | v | 10 | μm | 0.000847 |
| 15C | Circularity | Untreated | | v | 10 | μm | 0.000148 |
| 15D | Foci | Untreated | | v | 0.08 | μm | 9.61E−05 |
| 15D | Foci | 0.08 | μm | v | 0.4 | μm | 0.000856 |
| 15D | Foci | Untreated | | v | 0.4 | μm | 2.18E−05 |
| 15D | Area | Untreated | | v | 0.08 | μm | 0.001689 |
| 15D | Area | 0.08 | μm | v | 0.4 | μm | 0.00079 |
| 15D | Area | Untreated | | v | 0.4 | μm | 0.000158 |
| 15D | Intensity | Untreated | | v | 0.08 | μm | 3.5E−07 |
| 15D | Intensity | 0.08 | μm | v | 0.4 | μm | 0.000981 |
| 15D | Intensity | Untreated | | v | 0.4 | μm | 1.72E−07 |
| 15D | Circularity | Untreated | | v | 0.08 | μm | 0.000172 |
| 15D | Circularity | 0.08 | μm | v | 0.4 | μm | 0.00335 |
| 15D | Circularity | Untreated | | v | 0.4 | μm | 0.000131 |
| 15E | X-Gal | NS | | v | SEN | | 0.00036 |
| 15E | Ki67 | NS | | v | SEN | | 3.81E−05 |
| 15E | γH2A | NS | | v | SEN | | 0.003032 |
| 15E | Senescent % | Untreated | | v | 2 | μm | 0.038847 |
| 15E | Senescent % | 2 | μm | v | 10 | μm | 0.001916 |
| 15E | Senescent % | Untreated | | v | 10 | μm | 0.000152 |
| 15F | X-Gal | NS | | v | SEN | | 6.88E−05 |
| 15F | Ki67 | NS | | v | SEN | | 0.001453 |
| 15F | γH2A | NS | | v | SEN | | 0.00737 |
| 15F | Senescent % | Untreated | | v | 0.08 | um | 0.002625 |
| 15F | Senescent % | 0.08 | um | v | 0.4 | um | 0.014978 |

| | | | | | | |
|---|---|---|---|---|---|---|
| 15F | Senescent % | Untreated | v | 0.4 | um | 0.00058 |

| | | | | | |
|---|---|---|---|---|---|
| 19D | Young | Uninjured | v | 3DPI | 3.9E−06 |
| 19D | Young | Uninjured | v | 21DPI | 0.36828 |
| 19D | Aged | Uninjured | v | 3DPI | 2.34E−05 |
| 19D | Aged | Uninjured | v | 21DPI | 0.366969 |
| 19D | Geriatric | Uninjured | v | 3DPI | 0.001041 |
| 19D | Geriatric | Uninjured | v | 21DPI | 0.151178 |
| 19D | 3DPI | Young | v | Aged | 0.000522 |
| 19D | 3DPI | Aged | v | Geriatric | 0.159686 |
| 19D | 3DPI | Young | v | Geriatric | 0.001391 |
| 19G | Young | Uninjured | v | 3DPI | 2.82E−06 |
| 19G | Young | Uninjured | v | 21DPI | 0.125069 |
| 19G | Aged | Uninjured | v | 3DPI | 0.000266 |
| 19G | Aged | Uninjured | v | 21DPI | 0.117462 |
| 19G | Geriatric | Uninjured | v | 3DPI | 1.48E−05 |
| 19G | Geriatric | Uninjured | v | 21DPI | 0.074126 |
| 19G | 3DPI | Young | v | Aged | 0.089146 |
| 19G | 3DPI | Aged | v | Geriatric | 0.004067 |
| 19G | 3DPI | Young | v | Geriatric | 0.00028 |
| 22B | Ki67 (box) | Young | v | Geriatric | 0.000462 |
| 22B | Ki67 (violin) | Young | v | Geriatric | 0.033050 |
| 22C | Ki67 | Sen | v | NS | 0.00064 |
| 22E | γH2A (box) | Young | v | Geriatric | 0.003642 |
| 22E | γH2A (violin) | Young | v | Geriatric | 0.014191 |
| 22F | γH2A | Sen | v | NS | 0.021021 |

### REFERENCES

The following publications are incorporated herein by reference in their entirety.
Ahmad, A. & Dey, L. A k-mean clustering algorithm for mixed numeric and categorical data. Data Knowl. Eng. 63, 503-527 (2007).
Baghdadi, M. B. & Tajbakhsh, S. Regulation and phylogeny of skeletal muscle regeneration. Dev. Biol. 433, 200-209 (2018).
Chen, Ozanne, J.-H. and, Hales, S. E. and & Nicholas, C. Methods of Cellular Senescence Induction Using Oxidative Stress. 179-189 (2007) doi:10.1007/978-1-59745-361-5_14.
Chikenji, T. S. et al. p16INK4A-expressing mesenchymal stromal cells restore the senescence-clearance-regeneration sequence that is impaired in chronic muscle inflammation. EBioMedicine 44, 86-97 (2019).
Coppé, J.-P., Desprez, P.-Y., Krtolica, A. & Campisi, J. The Senescence-Associated Secretory Phenotype: The Dark Side of Tumor Suppression. Pathol. Mech. Dis. 5, 99-118 (2010).
Coryell, P. R., Diekman, B. O. & Loeser, R. F. Mechanisms and therapeutic implications of cellular senescence in osteoarthritis. Nat. Rev. Rheumatol. 17, 47-57 (2021).
Duran, I. et al. Detection of senescence using machine learning algorithms based on nuclear features. Nat. Commun. 15, 1041 (2024).
Ferro, A. et al. Blue intensity matters for cell cycle profiling in fluorescence DAPI-stained images. Lab. Investig. 97, 615-625 (2017).
Freyter, B. M. et al. Nuclear Fragility in Radiation-Induced Senescence: Blebs and Tubes Visualized by 3D Electron Microscopy. Cells 11, 273 (2022).
Grosse, L. et al. Defined p16High Senescent Cell Types Are Indispensable for Mouse Healthspan. Cell Metab 32, 87-99.e6 (2020).
Heckenbach, I. et al. Nuclear morphology is a deep learning biomarker of cellular senescence. Nat. Aging 2, 742-755 (2022).
Hu, L. et al. Why Senescent Cells Are Resistant to Apoptosis: An Insight for Senolytic Development. Front. Cell Dev. Biol. 10, 822816 (2022).
Huang, W., Hickson, L. J., Eirin, A., Kirkland, J. L.& Lerman, L. O. Cellular senescence: the good, the bad and the unknown. Nat. Rev. Nephrol. 18, 611-627 (2022).
Ieronimakis, N., Balasundaram, G. & Reyes, M. Direct Isolation, Culture and Transplant of Mouse Skeletal Muscle Derived Endothelial Cells with Angiogenic Potential. PLoS ONE 3, e1753 (2008).
Lane, N. et al. A phase 2, randomized, double-blind, placebo-controlled study of senolytic molecule UBX0101 in the treatment of painful knee osteoarthritis. Osteoarthr. Cartil. 29, S52-S53 (2021).
Lee, K.-A., Flores, R. R., Jang, I. H., Saathoff, A. & Robbins, P. D. Immune Senescence, Immunosenescence and Aging. Front. Aging 3, 900028 (2022).
Linders, A. N. et al. A review of the pathophysiological mechanisms of doxorubicin-induced cardiotoxicity and aging. npj Aging 10, 9 (2024).
Liu, L. et al. Impaired Notch Signaling Leads to a Decrease in p53 Activity and Mitotic Catastrophe in Aged Muscle Stem Cells. Cell Stem Cell 23, 544-556.e4 (2018).
Liu, L., Cheung, T. H., Charville, G. W. & Rando, T. A. Isolation of skeletal muscle stem cells by fluorescence-activated cell sorting. Nat. Protoc. 10, 1612-1624 (2015).
Manohar, S. et al. Genome homeostasis defects drive enlarged cells into senescence. Mol. Cell 83, 4032-4046.e6 (2023).
Maskey, R. S. et al. Sustained mTORC1 activity during palbociclib-induced growth arrest triggers senescence in ER+ breast cancer cells. Cell Cycle 20, 65-80 (2021).
Matias, I. et al. Loss of lamin-B1 and defective nuclear morphology are hallmarks of astrocyte senescence in vitro and in the aging human hippocampus. Aging Cell 21, e13521 (2022).
McCool, J. L., Hum, N. R., Sebastian, A. & Loots, G. G. Isolation of Murine Articular Chondrocytes for Single-Cell RNA or Bulk RNA Sequencing Analysis. Methods Mol. Biol. (Clifton, NJ) 2598, 187-196 (2022).
Moiseeva, V. et al. Senescence atlas reveals an aged-like inflamed niche that blunts muscle regeneration. Nature 613, 169-178 (2023).
Montecucco, A., Zanetta, F. & Biamonti, G. Molecular mechanisms of etoposide. EXCLI J. 14, 95-108 (2015).
Narita, M. et al. Rb-Mediated Heterochromatin Formation and Silencing of E2F Target Genes during Cellular Senescence. Cell 113, 703-716 (2003).
Nerstedt, A. & Smith, U. The impact of cellular senescence in human adipose tissue. J. Cell Commun. Signal. 17, 563-573 (2023).
Pospelova, T. V., Chitikova, Z. V. & Pospelov, V. A. An integrated approach for monitoring cell senescence. Methods Mol. Biol. (Clifton, NJ) 965, 383-408 (2013).
Quarta, M. et al. Bioengineered constructs combined with exercise enhance stem cell-mediated treatment of volumetric muscle loss. Nat. Commun. 8, 15613 (2017).
Rhinn, M., Ritschka, B. & Keyes, W. M. Cellular senescence in development, regeneration and disease. Development 146, dev151837 (2019).
Roger, L., Tomas, F. & Gire, V. Mechanisms and Regulation of Cellular Senescence. Int. J. Mol. Sci. 22, 13173 (2021).
Romanov, V. S. et al. p21Waf1 is required for cellular senescence but not for cell cycle arrest induced by the HDAC inhibitor sodium butyrate. Cell Cycle 9, 3945-3955 (2010).
Saito, Y., Chikenji, T. S., Matsumura, T., Nakano, M.& Fujimiya, M. Exercise enhances skeletal muscle regeneration by promoting senescence in fibro-adipogenic progenitors. Nat. Commun. 11, 889 (2020).
Sousa-Victor, P. et al. Geriatric muscle stem cells switch reversible quiescence into senescence. Nature 506, 316-321 (2014).
Stolarek, I., Samelak-Czajka, A., Figlerowicz, M. & Jackowiak, P. Dimensionality reduction by UMAP for visualizing and aiding in classification of imaging flow cytometry data. iScience 25, 105142 (2022).
Tuttle, C. S. L. et al. Cellular senescence and chronological age in various human tissues: A systematic review and meta-analysis. Aging Cell 19, e13083 (2020).
Valieva, Y., Ivanova, E., Fayzullin, A., Kurkov, A. & Igrunkova, A. Senescence-Associated β-Galactosidase Detection in Pathology. Diagnostics 12, 2309 (2022).
Wan, M., Gray-Gaillard, E. F. & Elisseeff, J. H. Cellular senescence in musculoskeletal homeostasis, diseases, and regeneration. Bone Res. 9, 41 (2021).
Wang, L., Lankhorst, L.& Bernards, R. Exploiting senescence for the treatment of cancer. Nat. Rev. Cancer 22, 340-355 (2022).
Wang, Z., Wei, D. & Xiao, H. Methods of cellular senescence induction using oxidative stress. Methods Mol. Biol. (Clifton, NJ) 1048, 135-144 (2013).
Wosczyna, M. N. & Rando, T. A. A Muscle Stem Cell Support Group: Coordinated Cellular Responses in Muscle Regeneration. Dev. cell 46, 135-143 (2018).
Wosczyna, M. N. et al. Mesenchymal Stromal Cells Are Required for Regeneration and Homeostatic Maintenance of Skeletal Muscle. Cell Rep. 27, 2029-2035.e5 (2019).
Wosczyna, M. N. et al. Targeting microRNA-mediated gene repression limits adipogenic conversion of skeletal muscle mesenchymal stromal cells. Cell Stem Cell 28, 1323-1334.e8 (2021).
Yagi, M., Endo, K., Komori, K. & Sekiya, I. Comparison of the effects of oxidative and inflammatory stresses on rat chondrocyte senescence. Sci. Rep. 13, 7697 (2023).
Zeng, W. et al. Restoration of CPEB4 prevents muscle stem cell senescence during aging. Dev. Cell 58, 1383-1398.e6 (2023).
Zhang, R. et al. Formation of MacroH2A-Containing Senescence-Associated Heterochromatin Foci and Senescence Driven by ASF1a and HIRA. Dev. Cell 8, 19-30 (2005).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A nontransitory computer readable medium storing instructions that, when executed by a processor, cause the processor to perform operations comprising:
obtaining at least one image comprising at least one animal cell;
pre-processing the at least one image;
segmenting the at least one pre-processed image to identify a region of the at least one image corresponding to a nucleus of the animal cell;
calculating at least one nuclear morphometric parameter of the animal cell from the region of the image;
providing the at least one nuclear morphometric parameter as an input to an unsupervised machine learning algorithm; and
calculating a senescence parameter of the at least one animal cell with the unsupervised machine learning algorithm.

2. The nontransitory computer readable medium of claim 1, wherein the at least one animal cell comprises a cell of the group consisting of: a stem cell, a muscle stem cell, a mesenchymal stem cell, a mesenchymal stromal cell, a fibroadipogenic progenitor, a endothelial cell, a skeletal muscle cell, a cartilage cell, a chondrocyte, an immune cell, a myoblast, an adipocyte, a preadipocyte, an epithelial cell, and a hepatocyte.

3. The nontransitory computer readable medium of claim 1, wherein the unsupervised machine learning algorithm comprises dimensional reduction.

4. The nontransitory computer readable medium of claim 3, wherein the dimensional reduction comprises UMAP.

5. The nontransitory computer readable medium of claim 1, wherein the unsupervised learning algorithm comprises cluster analysis.

6. The nontransitory computer readable medium of claim 5, wherein the cluster analysis is based on a value k, wherein k is greater than 2 and k defines the number of groups, wherein the number of groups comprise at least a senescence cluster and non-senescence cluster corresponding to the one or more animal cells.

7. The nontransitory computer readable medium of claim 1, wherein the at least one nuclear morphometric parameter is selected from the group consisting of: nuclear size, intensity of a nuclear stain, nuclear circularity, and dense foci.

8. The nontransitory computer readable medium of claim 1, wherein the at least one nuclear morphometric parameter comprises nuclear size, intensity of a nuclear stain, nuclear circularity, and heterochromatic foci.

9. The nontransitory computer readable medium of claim 1, wherein the pre-processing step comprises removing noise from the images using 3D deconvolution.

10. The nontransitory computer readable medium of claim 1, wherein the pre-processing step comprises generating a binary image from the at least one image.

11. The nontransitory computer readable medium of claim 1, wherein the senescence parameter comprises a senescence score.

12. The nontransitory computer readable medium of claim 1, wherein the senescence parameter comprises assigning the at least one animal cell to one of a set of discrete groups.

13. The nontransitory computer readable medium of claim 12, wherein the discrete groups are calculated based on k-means cluster analysis.

14. The nontransitory computer readable medium of claim 1 wherein the at least one animal cell was treated with a nucleic acid marker.

15. The nontransitory computer readable medium of claim 14, wherein the nucleic acid marker comprises DAPI.

16. The nontransitory computer readable medium of claim 1, wherein the step of segmenting the at least one pre-processed image to identify the region of the at least one image corresponding to the nucleus of the animal cell comprises calculating a size, a circularity, or a blebbing of at least one segment of the pre-processed image.

17. The nontransitory computer readable medium of claim 1, wherein the image is of a cell culture or a tissue sample.

18. The nontransitory computer readable medium of claim 1, wherein the image is of a tissue section.

19. A method of calculating a senescence score of at least one animal cell, comprising:
obtaining at least one image comprising at least one animal cell;
identifying a region of the at least one image corresponding to a nucleus of the animal cell;
calculating at least one nuclear morphometric parameter of the animal cell from the region of the image;
providing the at least one nuclear morphometric parameter as an input to an unsupervised machine learning algorithm; and
calculating a senescence parameter of the at least one animal cell with the unsupervised machine learning algorithm.

20. A method of screening a drug candidate comprising the steps of:
providing a culture comprising one or more animal cells;
calculating a first set of senescence parameters of at least one animal cell of the one or more animal cells of the culture via the method of claim 19;
contacting a drug candidate to the culture;
calculating a second set of senescence parameters of at least one animal cell of the one or more animal cells of the culture via the method of claim 19; and
comparing the first set of senescence parameters to the second set of senescence parameters.


SEQUENCE LISTING (20 sequences)

SEQ ID NO: 1 - DNA, 23 bp, synthetic construct
  source 1..23: mol_type = other DNA
taaaaacctggatcggaaccaaa

SEQ ID NO: 2 - DNA, 23 bp, synthetic construct
  source 1..23: mol_type = other DNA
gcattagcttcagatttacgggt

SEQ ID NO: 3 - DNA, 19 bp, synthetic construct
  source 1..19: mol_type = other DNA
cctggtgatgtccgacctg

SEQ ID NO: 4 - DNA, 19 bp, synthetic construct
  source 1..19: mol_type = other DNA
ccatgagcgcatcgcaatc

SEQ ID NO: 5 - DNA, 19 bp, synthetic construct
  source 1..19: mol_type = other DNA
caggcggtgcctatgtctc

SEQ ID NO: 6 - DNA, 22 bp, synthetic construct
  source 1..22: mol_type = other DNA
cgatcaccccgaagttcagtag

SEQ ID NO: 7 - DNA, 19 bp, synthetic construct
  source 1..19: mol_type = other DNA
atggcacccgaatggtcag

SEQ ID NO: 8 - DNA, 23 bp, synthetic construct
  source 1..23: mol_type = other DNA
ctccagtgtatagcctctctctg

SEQ ID NO: 9 - DNA, 21 bp, synthetic construct
  source 1..21: mol_type = other DNA
ctgcaagagacttccatccag

SEQ ID NO: 10 - DNA, 23 bp, synthetic construct
  source 1..23: mol_type = other DNA
agtggtatagacaggtctgttgg

SEQ ID NO: 11 - DNA, 19 bp, synthetic construct
  source 1..19: mol_type = other DNA
cctggtcccatgccaaacc

SEQ ID NO: 12 - DNA, 23 bp, synthetic construct
  source 1..23: mol_type = other DNA
cccttgaaagcataagtcttccc

SEQ ID NO: 13 - DNA, 20 bp, synthetic construct
  source 1..20: mol_type = other DNA
ccacctgcaagaccatcgac

SEQ ID NO: 14 - DNA, 21 bp, synthetic construct
  source 1..21: mol_type = other DNA
ctggcgagccttagtttggac

SEQ ID NO: 15 - DNA, 21 bp, synthetic construct
  source 1..21: mol_type = other DNA
gagtatgaggcggcactaaac

SEQ ID NO: 16 - DNA, 22 bp, synthetic construct
  source 1..22: mol_type = other DNA
catctgctaactgctttttggc

SEQ ID NO: 17 - DNA, 20 bp, synthetic construct
  source 1..20: mol_type = other DNA
tgcaggagacaagttctgga

SEQ ID NO: 18 - DNA, 20 bp, synthetic construct
  source 1..20: mol_type = other DNA
gacggcatccaggttatcag

SEQ ID NO: 19 - DNA, 20 bp, synthetic construct
  source 1..20: mol_type = other DNA
tcaagaaggtggtgaagcag

SEQ ID NO: 20 - DNA, 20 bp, synthetic construct
  source 1..20: mol_type = other DNA
gttgaagtcgcaggagacaa